(19) United States
(12) Patent Application Publication
(10) Pub. No.: US 2018/0346981 A1
(43) Pub. Date: Dec. 6, 2018

(54) Panel-based Genetic Diagnostic Testing for Inherited Eye Diseases

(71) Applicant: Massachusetts Eye and Ear Infirmary, Boston, MA (US)

(72) Inventors: Eric A. Pierce, Brookline, MA (US); Mark B. Consugar, Watertown, MA (US)

(21) Appl. No.: 15/527,918

(22) PCT Filed: Nov. 20, 2015
(86) PCT No.: PCT/US2015/061956

§ 371 Date: May 18, 2017

Related U.S. Application Data
(60) Provisional application No. 62/082,522, filed on Nov. 20, 2014.

(51) Int. Cl. C12Q 1/6883

(52) U.S. Cl. CPC C12Q 1/6883; C12Q 2600/156

(57) ABSTRACT

Methods of detecting a mutation associated with a genetic eye disease in a subject, using target enrichment and next generation sequencing.

### CLAIM OF PRIORITY

This application claims the benefit of U.S. Patent Application Ser. No. 62/082,522, filed on Nov. 20, 2014. The entire contents of the foregoing are hereby incorporated by reference.

### FEDERALLY SPONSORED RESEARCH OR DEVELOPMENT

This invention was made with Government support under Grant Nos. EY012910 and P3OEY014104 awarded by the National Institutes of Health. The Government has certain rights in the invention.

### TECHNICAL FIELD

Described herein are methods of detecting a mutation associated with a genetic eye disease in a subject, using target enrichment and next-generation sequencing.

### BACKGROUND

Inherited eye disorders are important causes of vision loss. IRDs are among the most common causes of blindness in working age people¹¹, and glaucoma is a leading cause of irreversible blindness worldwide^{12,13}. Genetic diagnostic testing for these disorders is challenging due to their genetic heterogeneity. For example, mutations in over 200 genes can cause IRDs (complete gene list available on the web at sph.uth.edu/retnet/), and multiple genes are known to underlie inherited forms of glaucoma and optic atrophy¹⁴⁻¹⁶.

### SUMMARY

Next-generation sequencing (NGS) based methods are being adopted broadly for genetic diagnostic testing, but the performance characteristics of these techniques have not been fully defined with regard to test accuracy and reproducibility. Described herein is a targeted enrichment and NGS approach for genetic diagnostic testing of patients with inherited eye disorders, including inherited retinal degenerations, optic atrophy and glaucoma. We performed experiments to measure the sensitivity, specificity, reproducibility as well as the clinical sensitivity of one embodiments of the test, the Genetic Eye Disease (GEDi) test as described herein. The GEDi test was highly reproducible and accurate, with sensitivity and specificity for single nucleotide variant detection of 97.9% and 100%, respectively. The sensitivity for variant detection was notably better than the 88.3% achieved by whole exome sequencing (WES) using the same metrics, due to better coverage of targeted genes in the GEDi test compared to commercially available exome capture sets. Prospective testing of 192 patients with IRDs indicated that the clinical sensitivity of the GEDi test is high, with a diagnostic rate of 51%.

Thus, in a first aspect provided herein are methods for detecting a genetic variation, e.g., a mutation, in a genomic region associated with a genetic disease, e.g., a genetic eye disease, in a subject (e.g., a mammalian, preferably human, subject). The methods include contacting a sample comprising fragmented genomic DNA (gDNA) from the subject with a plurality of bait ribonucleotides, wherein each bait binds to (forms a complex with) a fragment of gDNA comprising a genomic target sequence that is within a genomic region associated with a genetic eye disease, e.g., within 5-200 nucleotides of a selected gene, region or mutation associated with a genetic eye disease; the plurality of baits comprises sufficient baits to sequence each target sequence is sequenced at least 10 times; and the plurality comprises baits that bind to mutations in at least 100, 150, 200, or 230 genes, e.g., genes listed in Table 1a, 11, or 12; enriching the sample for the bait/gDNA complexes; isolating the gDNA fragments; determining the sequences of the isolated gDNA fragments using next generation sequencing, wherein each selected mutation is sequenced at least 10 times (i.e., a Depth of Coverage of at least 10×); and comparing the determined sequences to corresponding reference sequences, thereby detecting the presence of genetic variations, e.g., mutations, in a genomic region associated with genetic eye disease in the subject.

In some embodiments, the plurality of bait oligonucleotides comprises at least 10,000 oligonucleotides, e.g., at least 15,000 or at least 18,000 bait oligonucleotides, e.g., at least 10K, 15K, 18K, or all of the bait oligonucleotides listed in Table 11.

In some embodiments, the mutations include one or more (e.g., two, three, four, five, ten, 20, 30, 40, 50 or more) mutations listed in Tables 2, 3, 4, 9, or 10.

In some embodiments, the reference sequences include the normal (i.e., wild-type or non-mutated sequence, or non-disease associated) sequence, and wherein differences between the subject's genomic sequences and normal reference sequences indicate the presence of mutations, e.g., disease causing mutations, and identity between the subject's genomic sequences and normal reference sequences indicate the absence of mutations, e.g., the absence of disease causing mutations.

In some embodiments, the reference sequences include the mutant (i.e., a known mutation associated with eye disease) sequences, and wherein identity between the subject's genomic sequences and mutant reference sequences indicate the presence of mutations, e.g., disease causing mutations, and differences between the subject's genomic sequences and mutant reference sequences indicate the absence of mutations, e.g., the absence of disease causing mutations.

Depth of coverage is determined by the number of times a region of interest is sequenced, counting duplicate reads only once. See FIGS. 6A-6C for a schematic illustration. In some embodiments, sequence of each selected mutation at least 10 times (i.e., a Depth of Coverage of at least 10×) is obtained by one or more of: (1) stochastically increasing the number of baits per target; (2) near-target capture, using bait sequences up to 75 bp away from the mutation of interest; and/or (3) tiling the baits such that numerous overlapping baits target the same region.

In another aspect, kits are provided herein for use in a method described herein, e.g., for detecting a mutation in a genomic region associated with a genetic eye disease in a subject. The kits can include a plurality of bait ribonucleotides, wherein each bait is complementary to a genomic target sequence that is within a genomic region associated with a genetic eye disease, e.g., within 5-200 nucleotides of a selected gene, region or mutation associated with a genetic eye disease; the plurality of baits comprises sufficient baits to sequence each target sequence is sequenced at least 10 times; and the plurality comprises baits that bind to regions in at least 100, 150, 200, or 230 genes, e.g., genes listed in Table 1a, 11, or 12; reagents for enriching the sample for the bait/gDNA complexes; reagents for isolating the gDNA fragments; and reagents for determining the sequences of the isolated gDNA fragments using next generation sequencing.

In some embodiments, the plurality of bait oligonucleotides comprises at least 10,000 oligonucleotides, e.g., at least 15,000 or at least 18,000 bait oligonucleotides, e.g., at least 10K, 15K, 18K, or all of the bait oligonucleotides listed in Table 11.

In some embodiments, the target regions include one or more (e.g., two, three, four, five, ten, 20, 30, 40, 50 or more) genes or regions listed in Tables 2, 3, 4, 9, or 10.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### REFERENCE TO SEQUENCE LISTING

This application includes a sequence listing filed herewith electronically. The entire content of this file is hereby incorporated by reference.

### DESCRIPTION OF DRAWINGS

FIGS. 1A-B. (A) Analysis of empiric GEDi data shows that design gaps ≤75 bp were relatively well covered by “near-target” capture. (B) Representative Depth-of-Coverage (DoC) plot for a 12×-multiplexed capture sample using the custom GEDi targeted enrichment kit manufactured by Agilent Technologies, Inc. and 2×121 bp paired-end sequenced using a MiSeq next-generation sequencing (NGS) instrument manufactures by Illumina, Inc.

FIGS. 2A-C. (A) Integrative Genomics Viewer (IGV) screenshot of representative GEDi NGS validation data at chr15:78397352. The Omni 2.5 SNP data were determined to be incorrect in all samples due to a single base deletion adjacent to the interrogated base that shifted the analyzed base. (B) IGV screenshot of the putative c.1028T>G mutation of FSCN2 in OGI-267-573, clarifying the false positive variant call was due to misalignment of some NGS sequencing reads. (C) GEDi vs. Omni 2.5 concordance histogram plot corresponding to the 2,443 shared SNPs between the GEDi design and Omni 2.5 SNP microarray design for all 36 replicates of the 4 validation samples used in this study. KEY: MATCH-13 All GEDi NGS replicates matched Omni 2.5 SNP data; NO CALL-13 no NGS result; NO MATCH—≥1 NGS replicate did not match Omni 2.5 SNP data; OMNI NO VALUE—no Omni 2.5 SNP result; NO CALL/MATCH—≥1 NGS replicate had no result; all other NGS replicates matched Omni 2.5 SNP data.

FIG. 3. Comparison of V4+UTR WES and GEDi capture baits at the 5′-end of ABCC6. The ABCC6 reference used is a “collapsed” reference that accounts for all known gene isoforms.

FIG. 4. SAMtools view of incorrect Platinum alignments. View of individual GEDi sequence reads at position chr17:21311916 showing 4 haplotypes (numbered on right), consistent with incorrect alignment of at least some of the reads to this location.

FIGS. 5A-E Fundus Photos. A, B. Fundus photos for subject OGI-035-090, a male proband with a clinical diagnosis of cone dysfunction syndrome. Fundus exam showed normal optic nerves (ON) and retinal blood vessels, with bull's eye patterns detected in the maculae (arrows; panels A, C). The retinal peripheries were normal (not shown). C, D. Fundus photos for subject OGI-019-047, a female who presented at age 5 with peripheral field loss and nyctalopia. Fundus exam showed regions of chorioretinal atrophy (*) near the vascular arcades and optic nerves. ON, optic nerve. E, F. Fundus photos for subject OGI-314-749, a 31-year old with a history of renal disease and RP. Fundus exam showed pigment mottling in the maculae (arrows), early atrophic changes in the retina, with bone spicule pigmentation noted (arrow heads). ON, optic nerve.

FIGS. 6A-6C are schematics illustrating the concept of Depth of Coverage (DoC) and Breadth of Coverage (BoC). In 6A, 16 different sequencing reads included the boxed nucleotide, so the DoC in this case is 16. In 6B, 5 duplicate reads are present; they are all counted as one, so the DoC in this case is 12. In 6C, the Breadth of Coverage=100%, and the average DoC≅5×.

### DETAILED DESCRIPTION

It is increasingly desirable to obtain genetic diagnoses for patients with genetic eye disorders, as this information can influence patient care by both informing genetic risk assessment and identifying patients who would benefit from novel gene-based therapies¹⁷⁻²¹.

Next-generation sequencing (NGS) based testing methods are increasingly being used for genetic diagnostic testing. This is especially true for genetically heterogeneous disorders, such as inherited retinal degenerations (IRDs), hearing loss, cardiomyopathies, mitochondrial disorders and cancer¹⁻⁴. There are multiple advantages to these approaches, including the ability to simultaneously sequence many genes and to quantify allele frequency^{1,3,5,6}. The Next-generation Sequencing: Standardization of Clinical Testing (Nex-StoCT) workgroup of the CDC and the American College of Medical Genetics and Genomics (ACMG) have issued guidelines for clinical laboratory standards for NGS-based testing methods, which include determination of test accuracy, analytical sensitivity and specificity, reproducibility and repeatability^{7,8}. Defining these test characteristics is important, and despite the increased use of NGS-based tests in Clinical Laboratory Improvement Amendments (CLIA) and/or College of American Pathologists (CAP) certified laboratories^{3,6,9}, the sensitivity, specificity and reproducibility of these techniques have been defined for only a small subset of tests^{1,5,10}.

We developed a targeted enrichment and NGS approach for genetic diagnostic testing of patients with inherited eye disorders, including IRDs, optic atrophy and glaucoma, with improved accuracy, sensitivity and specificity, reproducibility and repeatability and/or clinical sensitivity as compared to previously reported uses of NGS techniques for genetic diagnostic testing of patients with IRDs, optic atrophy, and glaucoma²²⁻²⁶.

Our results suggest that selective targeted enrichment and NGS is the preferred method for diagnostic testing, especially for genetically heterogeneous disorders such as IRDs. The GEDi test has improved sensitivity when compared to WES while maintaining nearly perfect specificity. Our results show that the higher sensitivity of the GEDi test is due to improved probe design compared to the commercially available V4+UTR exome capture set (Agilent Technologies, Inc.) where probes were missing for approximately 10% of the regions targeted by the GEDi test. While the concept that targeted sequencing can out-perform standard exome sequencing based on better coverage has been discussed in reviews and commentaries regarding genetic diagnostic testing, only limited empiric comparisons of these two approaches to genetic diagnostic testing have been previously reported^{3,10,34}. Thus, while WES is now available as a clinical diagnostic test at some centers, and reports of using WES for diagnostic testing have been published, quantitation of the performance characteristics of the GEDi test makes it possible to identify and quantify the advantages of selective targeted enrichment over WES^{6,35,36} .

There are additional advantages of selective enrichment (or panel tests) over WES for diagnostic testing. The turn-around-time for the GEDi panel test run on a MiSeq NGS platform is approximately 1 day, which is considerably less than WES samples run on a HiSeq 2000 instrument (˜12 days). The current costs of selective exon capture tests are also lower than WES, although it is likely that this difference will continue to diminish over time. At present (August 2014), the cost of the materials needed for GEDi testing per patient is approximately $430, compared to $1,325 per patient for WES using the sequence depth described. In addition, panel testing has a higher pre-test probability of finding a meaningful result, and reduces the potential for making incidental sequence findings, which can be challenging for both health care providers and patients^{37,38}.

Some hybridization-based capture approaches are subject to limitations by “design gaps”: genomic regions where it is not possible to design targeted enrichment probes. Specifically, design gaps are commonly associated with genome regions with high GC content and/or repetitive nonunique elements that can be resistant to accurate capture probe design³. Fortunately, based on the data obtained for the GEDi test, near-target sequence coverage limits this problem to gaps larger than 75 bp, which significantly reduces this problem. It is also possible to use alternative approaches to capture regions in hybridization design gaps, including amplification-based strategies such as Agilent Technologies' HaloPlex technique³⁹.

Sufficient sequence depth is also needed to make accurate base calls from the NGS data. For GEDi we showed that a minimum depth of coverage of 10× gave a specificity of 100%. Even with sufficient depth of coverage, misalignment of short NGS reads can lead to incorrect base identification, especially for repetitive regions or genes with paralogous copies elsewhere in the genome, which we observed in both the GEDi and WES data. Until longer sequencing reads become routinely available, this is likely to remain a problem; however, familial segregation studies and Sanger validation of likely pathogenic alleles can be useful to resolve these discrepancies.

We carefully evaluated the overall performance characteristics of the GEDi test and showed that the test is both sensitive and specific and is highly reproducible and accurate. Thorough analyses of these test characteristics have been reported for one other NGS-based diagnostic test, a targeted enrichment and NGS-based test for 25 genes associated with cancer called the WUCaMP assay⁵. The sensitivity and specificity of the WUCaMP assay were determined by comparing test data with WGS data from Complete Genomics for HapMap sample NA19240. For these studies, the test samples were sequenced to a high depth of coverage, with 96.9% of the targeted regions covered at ≥50× depth. The reported sensitivity and specificity for detecting SNVs were 98.3% and 100%, respectively⁵. The sensitivity and specificity of the GEDi test for SNV detection are comparable, at 96.4% to 97.9% and 99.9% to 100%, respectively, with an overall accuracy for both the SNPs and indels of 99.9%. We also showed that the GEDi test is highly repeatable and reproducible, with kappa statistics of 0.83088 and 0.76366, respectively, indicating excellent agreement between the data obtained in the replicate testing of the 4 individual DNA samples³².

The clinical sensitivity of the GEDi test was 51% in patients with IRDs, a rate that is consistent with prior reports²²⁻²⁶. It is hypothesized that subjects without mutations in GEDi target genes must have mutations in novel disease genes, or in non-coding portions of the currently identified IRD disease genes. Exome and genome sequencing will be required for identifications of these mutations. It is also possible that some subjects have mutations that cannot be readily detected by sequencing-based approaches, such as copy number variations (CNVs)^{25,40}.

Comprehensive genetic diagnostic testing for genetically and phenotypically heterogeneous disorders such as IRDs can also lead to diagnoses outside of the reported genotype-phenotype relationships. Seven of the patients with genetic diagnoses had atypical phenotypic features confirming that it can be difficult to predict the genetic cause of disease based on clinical findings alone²⁶.

In summary, the GEDi test offers a number of advantages as a clinical diagnostic test for patients with inherited eye disorders. Given the potential for gene-based therapies for inherited disorders in general, and inherited eye disorders specifically, genetic diagnostic testing will increasingly be necessary for optimal care of patients with genetic diseases. Further, the GEDi test statistics make a strong case for the use of targeted tests in the clinical setting, as they are highly accurate, reproducible and have better overall performance than more general tests such as conventional WES analyses.

Methods of Detecting Genetic Eye Disease

The methods described herein can be used to identify, and optionally treat, subjects with genetic eye disease. The methods include obtaining a sample comprising genomic DNA from a subject, e.g., a subject suspected of having a genetic eye disease. Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine or expectorant. The sample itself will typically include nucleated cells (e.g., blood or buccal cells) or tissue removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al., 2003, supra. The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction, or cell lysis followed by salt-precipitation methods. Alternatively, genomic DNA can be extracted with kits such as the QIAamp® Tissue Kit (Qiagen, Chatsworth, Calif.) and the Wizard® Genomic DNA purification kit (Promega). In some embodiments, the sample is blood, e.g., venous blood, and a method in which the cells are lysed with an anionic detergent in the presence of a DNA stabilizer and then a modified salting-out precipitation method is used for purification of DNA, e.g., with the Gentra Puregene Blood Kit (Qiagen).

The genomic DNA (gDNA) is then optionally fragmented, e.g., using mechanical (e.g., repeatedly passing the sample through a needle), high-performance liquid chromatography (HPLC) pump, nebulization, sonication, or hydrodynamic shearing using a filter screen with uniform pores. In preferred embodiments, the gDNA is fragmented into random pieces about 150-300 nts in length, e.g., about 200-250 nts in length, e.g., using a Covaris E220 ultrasonicator tuned to shear the gDNA to between 200-250 bp.

The fragmented DNA is then subjected to targeted enrichment, wherein fragments comprising sequences of interest are captured using complementary RNA bait sequences. See, e.g., Gnirke et al., Nat Biotechnol. 2009 February; 27(2):182-9. The baits are about 100-150 nts long, e.g., 115-125 bp, preferably 120 bp long, and are complementary to coding and select intronic regions, e.g., regions within 10-200, e.g., 10-75, nucleotides of a selected gene or intron that is known or suspected to be associated with eye disease (e.g., associated with IRD, optic atrophy, and glaucoma disease genes (described/listed previously). Such mutations can be identified, e.g., by reference to prior publication, or by empirical observation, and can include those regions described herein in Table 11. The baits provide sufficient coverage that every base within the targeted regions can be sequenced to a depth of coverage (DoC) of at least 10×, and in some embodiments provide coverage of the genes' entire coding regions. The baits are preferably identified by a method described herein and sufficient DoC (e.g., at least 10×) is obtained for every region of interest, e.g., in regions in which a design gap exists, by one or more of (1) stochiometrically increasing the number of baits per target region; (2) near-target capture, using bait sequences up to 75 bp adjacent to the region of interest; and (3) tiling the baits such that numerous overlapping baits target the same region.

The bait/gDNA complexes are isolated from the sample using methods known in the art, e.g., as described in Gnirke et al., Nat Biotechnol. 2009 February; 27(2):182-9. For example, the bait can be labeled with a bead such as a magnetic bead, or can be bound to a surface such as a bead, e.g., a magnetic bead, using known methods. For example, biotin-conjugated capture probes can be bound onto a streptavidin-coated surface, e.g., a streptavidin coated beads, e.g., magnetic beads. Fragments not containing sequences of interest (i.e., not bound to a bait) are removed from the sample, e.g., by washing, and/or fragments containing sequences of interest (i.e., bound to a bait) are removed from the sample, e.g., by concentration using an appropriate method, e.g., magnetism. The bait/gDNA complexes are then dissociated, e.g., using high salt conditions.

The gDNA fragments are then sequenced in parallel (e.g., are all sequenced at substantially the same time), preferably using massively parallel next-generation DNA sequencing methods. A number of such methods are known, and include Illumina sequencing (e.g., using a HiSeq or MiSeq system), Roche 454 sequencing, Ion torrent and Ion proton sequencing. The sequences are analyzed and aligned using computer-based methods to determine the subject's genomic sequence, and compared to appropriate reference sequences, e.g., reference sequences comprising areas with known mutations associated with eye disease. The reference sequences can include the normal (i.e., wild-type or non-mutated sequence, or non-disease associated sequence) and/or the mutant (i.e., a known mutation associated with eye disease) sequences. In some embodiments, the normal sequences are obtained from the most recent version of the reference genome, e.g., as described in Falk et al., Nature Genetics. Jul. 29, 2012; 44(9):1040-1045. Differences between the subject's genomic sequences and normal reference sequences indicate the presence of genetic mutations (i.e., variants), of which some variants are disease-causing mutations. Identity between the subject's genomic sequences and mutant reference sequences indicate the presence of mutations, e.g., disease causing mutations. Differences between the subject's genomic sequences and mutant reference sequences indicate the absence of mutations, e.g., the absence of disease causing mutations. Identity between the subject's genomic sequences and normal reference sequences indicate the absence of mutations, e.g., the absence of disease causing mutations.

Once a subject is identified as having one or more mutations known to be associated with eye disease, the subject can be treated for the disease. For example, a subject who is identified as having a genetic eye disease can be treated using gene therapy to correct the identified defect (e.g., as described in Maguire et al., New England Journal of Medicine. May 22, 2008; 358(21):2240-2248), or can be treated using conventional pharmacological or surgical methods for the disease detected.

For some patients with inherited retinal degenerations, high dose vitamin A supplementation may reduce the rate of disease progression (Berson et al., Arch Ophthalmol. 2012 Feb. 13). See, e.g., Pierce, Masland, Miller, eds. Retinal Disorders: Genetic Approaches to Diagnosis and Treatment. Cold Spring Harbor: Cold Spring Harbor Laboratory Press, 2014.

Kits

Also within the scope of the invention are kits comprising baits that hybridize with regions of genes as described herein and can be used to detect a mutation described herein. The kit can include one or more other elements including: instructions for use; and other reagents, e.g., magnetic beads for isolating the bait/gDNA complexes from the sample. Instructions for use can include instructions for diagnostic applications of the probe for predicting response to treatment in a method described herein. Other instructions can include instructions and reagents for fragmenting gDNA, for isolating the bait/gDNA complexes, for dissociating the bait/gDNA complexes, and/or instructions for obtaining a sample to be analyzed from a subject.

Devices and reagents for use in obtaining the sample can also be provided. For example, test kits can include devices and instructions that a health care provider or a subject can use to obtain a sample, e.g., of sputum, buccal cells, or blood. For example, buccal cells can be obtained using a buccal swab or brush, or using mouthwash.

Kits as provided herein can also include a mailer, e.g., a postage paid envelope or mailing pack, that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms, e.g., the test requisition form, and the container holding the sample, can be coded, e.g., with a bar code, for identifying the subject who provided the sample.

In some embodiments, the kits can include one or more reagents for processing a sample. For example, a kit can include reagents for isolating gDNA from a sample. The kits can also, optionally, contain one or more reagents for performing next-generation sequencing of the target-enriched sample.

In some embodiments, the kits can include a software package for analyzing the results.

### EXAMPLES

An exemplary embodiment of the invention (the Genetic Eye Disease (GEDi) test) is further described in the following examples, which do not limit the scope of the invention described in the claims.

Materials and Methods

The following materials and methods were used in the examples set forth herein.

Patient Samples

The clinical study was approved by the institutional review boards of the University of Pennsylvania and the Massachusetts Eye and Ear Infirmary, and conformed to the tenets of the Declaration of Helsinki. Informed consent was obtained from all participants, who were recruited after having been identified to have a form of inherited retinal degeneration following clinical evaluation by EAP or ABF at Mass Eye and Ear or Children's Hospital Boston, respectively. Genomic DNA (gDNA) was extracted from patient blood using the PreAnalytiX (QIAGEN/BD Biosciences; Valencia, Calif.) PAXgene Blood DNA Kit (PAXgene Blood DNA Kit Handbook, October 2009) or DNAzol (Life Technologies, Carlsbad, Calif.).

Targeted Enrichment

i. Targeted Enrichment Bait Library Design

The custom SureSelect targeted enrichment GEDi capture kit (Agilent Technologies, Inc., Santa Clara, Calif.) was designed to capture and enrich coding exons, 5′-/3′-UTRs, and select deep intronic regions known to harbor pathogenic mutations, associated with the 214 known IRD disease genes described in the Retinal Information Network database (RetNet; sph.uth.edu/Retnet/) up to April 2013, as well as 8 early-onset glaucoma and optic atrophy genes, using Agilent Technologies' eArray web design tool (earny.chem.agilent.com/earray/). The GEDi capture kit also includes 24 candidate IRD disease genes, 9 age-related macular degeneration risk factor genes, and 1 non-syndromic hearing loss gene. Additional information regarding the parameters used for GEDi capture kit design is below. The custom mitochondrial genome targeted enrichment baits we designed previously were also included as part of the GEDi capture kit²⁷. Complete lists of the GEDi targeted genes and intronic regions are shown in Tables 1a and 1b, respectively.

ii. Capture Library Sample Preparation

Illumina-compatible paired-end/multiplexable GEDi targeted enrichment capture libraries were generated as described, using the following parameters: a) no less than 1.5 mcg of sheared gDNA was used for pre-capture library generation; b) 5-cycles of pre-capture PCR were used for all samples; c) no less than 400 ng of pre-capture library was used during bait hybridizations; d) 14-cycles of post-capture PCR were used to generate all capture libraries; e) all samples were post-capture indexed; and f) sample multiplex ratios were determined based on post-capture indexed sample concentrations (Agilent methods, part no.: G7530-90000; Protocol v2.1, May 2011).

NGS Analysis

GEDi targeted enrichment sample sequencing was performed on an Illumina MiSeq NGS platform (Illumina, Inc., San Diego, Calif.). A 12× patient sample multiplex was clustered to an average cluster density of between 750-900 K clusters per mm² and 121×6×121 bp indexed/paired-end analyzed using Illumina's 300 cycle MiSeq Reagent Kit V2.

Informatics Analyses

Analysis of the sequence data obtained was performed using a combination of publically available and custom software tools, as described²⁸. Briefly, BWA (version 0.6.2-r126) was used to align the sequence reads to the human reference genome used by the 1000 Genomes Project. SAMtools (version 0.1.18 or r982:295) was used to remove potential duplicates, and make initial SNP and indel calls, which were refined using a custom program²⁸. A coverage depth cutoff of 10× was applied. Resulting variant calls were annotated using our custom human bp codon resource²⁸. Custom scripts were also developed and used to identify candidate variants that fit different filtering criteria, such as genetic models. Variants that fit the appropriate inheritance patterns, and were rare based on data from the 1000 Genomes Project, the NHLBI Exome Sequencing Project Exome Variant Server, and our own internal controls were considered to be potentially pathogenic. See the Supplementary Methods section for additional information.

GEDi Capture Kit Design

To design the GEDi capture kit using Agilent Technologies' eArray web design tool (earray.chem.agilent.com/earray/) the RefSeq CDS exon and 5′-/3′-UTR coordinates corresponding to the genes in Table 1a were queried and downloaded from the UCSC Genome Bioinformatics site (genome.ucsc.edu/). Alternatively, the genomic coordinates corresponding to the deep intronic variants identified in Table 1b were expanded to include ±5 bp flanking either side of the variant (totaling 11 bp) prior to being included with the CDS exon and 5′-/3′-UTR gene coordinates for targeted enrichment bait design. Targeted enrichment coordinates were sorted into two groups: 1) those corresponding to targets ≥121 bp, and 2) those corresponding to targets ≤120 bp. The ≥121 bp group was submitted to eArray under the following design conditions: Platform: Illumina Long Read (75+ bp); Tiling: 2× and Justified; 5′-/3′-target flanking regions: ±10 bp; Masking: Moderately Stringent. The 120 bp group (i.e. “orphan” bait group; coordinate size is ≤the length of a 120 bp capture bait, so only one bait—an “orphan bait”—can be designed against the region) was submitted to eArray under the following design conditions: Platform: Illumina Long Read (75+ bp); Tiling: 1× and Centered; 5′-/3′-target flanking regions: ±20 bp; Masking: Moderately Stringent.

The resulting .fate files were sorted based primarily on the number of baits designed in the coordinate region and secondarily on the percentage of the coordinate region covered. Regions with zero baits designed and/or ≤75% covered were resubmitted to eArray according to the above design conditions save that the Masking function was turned off. In order to avoid “bait poisoning” during the capture reaction (i.e. preferential capture of non-unique DNA sequences versus the unique targeted regions), the uniqueness of the resulting non-masked baits were determined using UCSC's Batch BLAT tool. Baits that had a homology score (homology score bases match—(bases mismatch+#gaps))≥40 were included in the bait final bait group; those that did not were discarded. Finally, orphan bait replicates were increased two-fold as a means of avoiding a capture bias resultant from an imbalanced 2×-tiled region v. 1×-tiled region bait ratio (i.e. equivalent to the ≥121 bp tiling frequency of 2×). Additional information regarding the capture baits used in the GEDi test is in Table 11.

TABLE 1a
The list of genes and regions targeted in the GEDi capture set.

| | | | | | | | |
|---|---|---|---|---|---|---|---|
| ABCA4 | CAPN5 | DNAH10* | IFT80 | NDP | PDE6H | RGS9 | TPP1 |
| ABCC6 | CC2D2A | DNAH9* | IFT88* | NEK4* | PDZD7 | RGS9BP | TREX1 |
| ABHD12 | CDH23 | EFEMP1 | IKGKB* | NEK8 | PEX1 | RHO | TRIM32 |
| ADAM9 | CDH3 | ELOVL4 | IMPDH1 | NIM1* | PEX10 | RIMS1 | TRPC1* |
| AHI1 | CDHR1 | ELP4 | IMPG2 | NMNAT1 | PEX14 | RLBP1 | TRPM1 |
| AIPL1 | CEP164 | ERCC6^{@} | INPP5E | NPHP1 | PEX16 | ROM1 | TRPM2* |
| AKAP9* | CEP290 | EYS | INVS | NPHP3 | PEX19 | RP1 | TSPAN12 |
| ALMS1 | CEP41 | FAM161A | IQCB1 | NPHP4 | PEX2 | RP1L1 | TTC21B |
| APOE\hat{ } | CERKL | FBLN5^{@} | ITM2B | NR2E3 | PEX5 | RP2 | TTC8 |
| ARL13B | CFB^{@} | FLVCR1 | JAG1 | NRL | PEX6 | RP9* | TTPA |
| ARL6 | CFH | FOXC1⁺ | KCNJ12* | NUB1* | PEX7 | RPE65 | TULP1 |
| ARMS2^{@} | CHM | FSCN2 | KCNJ13 | NXNL1* | PGK1 | RPGR | TYR |
| ATXN7 | CIB2 | FZD4 | KCNV2 | NYX | PHYH | RPGRIP1 | TYRP1 |
| BBS1 | CLN3 | GJB2^{#} | KCTD7 | OAT | PITPNM3 | RPGRIP1L | UNC119 |
| BBS10 | CLN5 | GNAT1 | KIF11 | OCA2 | PITX2⁺ | RS1 | USH1C |
| BBS12 | CLN6 | GNAT2 | KLHL7 | OFD1 | PLA2G5 | SAG | USH1G |
| BBS2 | CLN8 | GNB1* | LCA5 | OPA1+ | PPT1 | SDCCAG8 | USH2A |
| BBS4 | CLRN1 | GNPTG | LRAT | OPA3 | PRCD | SEMA4A | VCAN |
| BBS5 | CNGA1 | GPR143 | LRIT3 | OPN1LW | PRKACA* | SLC24A1 | VPS13B |
| BBS7 | CNGA3 | GPR179 | LAPS | OPN1MW | PROM1 | SLC45A2 | WASF3* |
| BBS9 | CNGB1 | GPR98 | LTBP2⁺ | OPN1SW | PRPF3 | SLC4A5* | WDPCP |
| BEST1 | CNGB3 | GRK1 | LZTFL1 | OPTN⁺ | PRPF31 | SNRNP200 | WDR19 |
| C1QTNF5 | CNNM4 | GRM6 | MAK | OSMR* | PRPF6 | SPATA7 | WDR65* |
| C2^{@} | COL11A1 | GRN | MERTK | OTX2 | PRPF8 | TEAD1 | WFS1 |
| C2orf71 | COL2A1 | GUCA1A | MFN2 | PANK2 | PRPH2 | TIMM8A | ZNF423 |
| C3^{@} | COL9A1 | GUCA1B | MFRP | PAX2 | RAB28 | TIMP3 | ZNF513 |
| C5orf42 | CRB1 | GUCY2D | MFSD8 | PAX6⁺ | RAX2* | TLR3^{@} | |
| C8orf37 | CRX | HARS | MICALCL* | PCDH15 | RBP3 | TLR4^{@} | Mito- |
| CA4 | CTCF* | HMCN1^{@} | MKKS | PDE6A | RBP4 | TMEM126A | chondrial |
| | | | | | | | Genome |
| CABP4 | CYP1B1⁺ | HTRA1^{@} | MKS1 | PDE6B | RD3 | TMEM231 | |
| CACNA1F | CYP4V2 | IDH3B | MTTP | PDE6C | RDH12 | TMEM237 | |
| CACNA1H* | DFNB31 | IFT140 | MYO7A | PDE6D* | RDH5 | TMEM67 | |
| CACNA2D4 | DHDDS | IFT172 | MYOC⁺ | PDE6G | RGR | TOPORS | |

Notes:
⁺Early onset-Glaucoma and Optic Nerve Atrophy genes,
*inherited eye disease candidate gene,
^{@}AMD risk factor gene (genes that also are reported to have roles in inherited retinal disorders, such as ABCA4 and CFH are not included in this category),
^{#}non-syndromic hearing loss gene,
\hat{ }No longer in RetNet (previously identified as AMD risk factor gene).

TABLE 1b
GEDi Targeted Enrichment Deep Intronic Variant List

| | Accession | | | | |
|---|---|---|---|---|---|
| Gene | Number | Position | Mutation | Source | Reference |
| USH2A | NM_ 206933.2 | chr1:216064540 | c.7595 − 2144A > G | PubMed | Vache (2012) Hum |
| | | | | | Mutat 33, 104 |
| ERCC6 | NM_000124.2 | chr10:50681659 | c.2599 − 26T > C | HGMD | Laugel (2009) Hum |
| | | | | | Mutat 31, 113 |
| ERCC6 | NM_000124.2 | chr10:50681663 | c.2599 − 30A > G | HGMD | Troelstra (1992) |
| | | | | | Cell 71, 939 |
| BBS1 | NM_024649.4 | chr11:66291105 | c.951 + 58C > T | HGMD | Abu Safieh (2010) J |
| | | | | | Med Genet 47, 236 |
| MYO7A | NM_000260.3 | chr11:76893448 | c.3109 − 21G > A | HGMD | Janecke (1999) Hum |
| | | | | | Mutat 13, 133 |
| COL2A1 | NM_001844.4 | chr12:48370512 | c.3435 + 83G > C | HGMD | Richards (2012) Eur J |
| | | | | | Hum Genet 20, 552 |
| COL2A1 | NM_001844.4 | chr12:48370516 | c.3435 + 79T > A | HGMD | Richards (2012) Eur J |
| | | | | | Hum Genet 20, 552 |
| COL2A1 | NM_001844.4 | chr12:48380015 | c.1527 + 104A > C | HGMD | Richards (2012) Eur J |
| | | | | | Hum Genet 20, 552 |
| COL2A1 | NM_001844.4 | chr12:48379984 | c.1527 + 135C > T | HGMD | Richards (2006) Hum |
| | | | | | Mutat 27, 696 |
| CEP290 | NM_025114.3 | chr12:88494960 | c.2991 + 1655T > C | HGMD | den Hollander (2006) |
| | | | | | Am J Hum |
| | | | | | Genet 79, 556 |
| OFD1 | NM_003611.2 | chrX:13768358 | c.935 + 706A > G | PubMed | Webb (2012) Hum Mol |
| | | | | | Genet 15, 3647 |
| CHM | NM_000390.2 | chrX:85223644 | c.315 − 4587A > T | HGMD | van den Hurk (2003) |
| | | | | | H Genet 113, 268 |

TABLE 12
Subsets of genes related to genetic eye disease.

| Category | Genes (RefSeq Aliases (for UCSC); v5.0) |
|---|---|
| RetNet <= v2.1 | ABCA4; AHI1; AIPL1; ALMS1; ARL13B; ARL6; ATXN7; BBS1; |
| (FOUNDATION) Genes | BBS10; BBS12; BBS2; BBS4; BBS5; BBS7; BBS9; BEST1; |
| | C1QTNF5; C2ORF71; CA4; CABP4; CACNA1F; CACNA2D4; |
| | CC2D24; CDH23; CDH3; CEP164; CEP290; CERKL; CHM; |
| | CLN3; CLRN1; CNGA1; CNGA3; CNGB1; CNGB3; CNNM4; |
| | VPS13B; COL11A1; COL2A1; COL9A1; CRB1; CRX; CYP4V2; |
| | DFNB31; DHDDS; EFEMP1; ELOVL4; ERCC6; EYS; FAM161A; |
| | FLVCR1; FSCN2; FZD4; GNAT1; GNAT2; GNB1; GPR98; GRK1; |
| | GRM6; GUCA1A; GUCA1B; GUCY2D; IDH38; IFT80; IMPDH1; |
| | IMPG2; INVS; IQCB1; JAG1; KCNJ13; KCNV2; KLHL7; LCA5; |
| | LRAT; LRP5; MERTK; MFRP; NIM1; MKKS; MTTP; MYO7A; |
| | NDP; NEK4; NEK8; NPHP1; NPHP3; NPHP4; NR2E3; NRL; |
| | NUB1; NYX; OAT; OPA1; OPA3; OPN1LW; OPN1MW; |
| | OPN1SW; PANK2; PCDH15; CDHR1; PDE6A; PDE6B; PDE6G; |
| | PDZD7; PEX1; PEX10; PEX14; PEX16; PEX19; PEX2; PEX5; |
| | PEX6; PEX7; PHYH; PITPNM3; PRCD; PRKACA; PROM1; |
| | PRPF3; PRPF31; PRPF6; PRPF8; PRPH2; RAX2; RBP3; RBP4; |
| | RD3; RDH12; RDH5; RGR; RGS9; RGS9BP; RHO; RIMS1; |
| | RLBP1; ROM1; RP1; RP1L1; RP2; RP9; RPE65; RPGR; |
| | RPGRIP1; RPGRIP1L; RS1; SAG; SDCCAG8; SEMA4A; |
| | SLC24A1; SLC4A5; SNRNP200; SPATA7; TEAD1; TIMP3; |
| | TMEM126A; TMEM67; TOPORS; TRIM32; TRPC1; TRPM1; |
| | TRPM2; TSPAN12; TTC21B; TTC8; TULP1; UNC119; USH1C; |
| | USH1G; USH2A; VCAN; WFS1; ZNF513; CTCF; GPR143; |
| | OCA2; SLC45A2; TYR; TYRP1 |
| RetNet v2.2 UPDATE | C8orf37; GNPTG; MAK; PLA2G5; TMEM237; WDPCP; |
| | WDR19 |
| “RetNet v3.1 UPDATE | NMNAT1; CYP1B1; PAX6; FOXC1; PITX2; MYOC; OPTN; |
| (NMNAT1 & Glaucoma)” | OPA1 ; LTBP2 |
| RetNet v3.2 RetNet UPDATE | ABCC6; ADAM9; MKS1; OTX2; PAX2; PDE6C; TIMM8A; |
| | TREX1; TTPA; PDE6H; PDE6D; INPP5E; ABHD12; CAPN5; |
| | CIB2; NXNL1; LRIT3; WASF3; RAB28 |
| RetNet v4.0 UPDATE | MFN2; LZTFL1; HARS; KIF11; GPR179; OFD1; IFT140; IFT88; |
| | CEP41; C5orf42; ZNF423; TMEM231; PGKl; ITM2B; |
| | KIAA2026; WDR34; IFT172 |
| RetNetv 4.1 UPDATE-042513 | PPT1; TPP1; CLN5; CLN6; MFSD8; CLN8; KCTD7; GRN |

Whole Exome Sequencing

Whole exome targeted enrichment was performed with a BRAVO automated workstation (Agilent Technologies, Inc.; Santa Clara, Calif.) using the SureSelect Human V4+UTR+Mito All Exon targeted enrichment kit (Agilent) according to their standard automation protocol (Pub. No. G7550-90000, Version D.1, April 2012). This kit includes custom baits designed to capture/enrich the mitochondrial genome¹.

Next-Generation Sequencing (NGS) analysis was performed using an Illumina HiSeq 2000 NGS instrument (Illumina, Inc.; Carlsbad, Calif.). A 7 picoMolar (pM) 4-sample multiplex sample (i.e. 1.75 pM per capture library) was clustered in duplicate flow cell lanes at ˜750,000 clusters per millimeter-squared, followed by 101|7|101 bp paired-end indexed analysis.

Informatic Analyses

Following variant identification using the approach described herein, a custom program was used to further refute the SNP and indel calls. The custom program uses a false discovery rate approach to adjust raw base counts at a candidate position after Benjamini and Hochberg correction based on quality values of all bases². Resulting variant calls were annotated using our custom human bp codon resource (HBCR). The HBCR maps each base position in the human reference genome, based on Ensembl Release 65 gene annotations, to its corresponding, if any, transcripts, genes, codons, encoded amino acids, and translation frames. Additional annotations of each variant call were provided using data sets downloaded from the 1000 Genomes Project website (The 1000 Genomes Project Consortium, Nature 467,1061-1073(28 October 2010)); the NHLBI Exome Sequencing Project Exome Variant Server (EVS); and the UCSC Genome Browser³. These annotations include allele frequencies, SIFT, PolyPhen and Mutation Taster predictions, and phastCons conservation scores^{4,5}. Custom scripts were also developed and used to identify candidate variants that fit different filtering criteria, such as genetic models. The fraction of a variant base has to be between 0.25-0.75 to be called heterozygous and above 0.75 to be called homozygous. The variant frequency cutoff used was ≤0.15%, which is the frequency in the EVS of the most common recessive RP mutation in the USH2A gene [c.2276G>T; p.(Cys759Phe)]; mutations in USH2A are reported to be the most common cause of recessive RP⁶.

Variant Validation

All variants deemed likely pathogenic were validated via PCR and Sanger sequencing using standard methods^{3,7}. When possible/available, samples from proband family members were included to confirm segregation and/or bi-parental inheritance. PCR amplicons were between 350-650 bp and spanned the pathogenic variants in question. PCR products were sequenced with the BigDye Terminator v3.1 Cycle Sequencing Kit on ABI 3130×1 or 3730×1 DNA analyzers (Applied Biosystems, Foster City, Calif., USA). Sequence data were analyzed using LaserGene (DNASTAR, Inc., Madison, Wis.) and/or MacVector (MacVector, Inc., Cary, N.C.) software.

Single Nucleotide Polymorphism (SNP) Genotyping

Genomic DNA samples were prepared and hybridized using Illumina's Human Omni 2.5 v1.1 BeadChip (Omni 2 5) arrays according to standard methods (part no.: 15023139; Protocol Rev. A, April 2011). All bead arrays were analyzed using Illumina's iScan array scanner; SNP calls were made using the Genotyping module of Illumina's GenomeStudio data analysis software⁸.

Palmitoyl-Protein Thioesterase-1 (PPT1) Assay

Punches from blood spotted onto standard collection filter paper were used to assay for PPT1 activity. A one step assay using the fluorescent substrate 4-methylumbelliferyl-6-thiopalmitoyl-b-D-galactopyranoside was performed in triplicate after incubation for 20 hrs at 37° C. as previously described⁹. Fluorescence was measured on a Fluoro-Count (Packard) fluorimeter using filter settings for the released 4-methylumbelliferone (excitation 360 nm, emission 460 nm).

### Example 1. GEDi Test Design

Probes were designed for 257 genes targeted by the GEDi selective capture system as well as the mitochondrial genome, since retinal degeneration and optic atrophy can accompany mitochondrial disease²⁷. Probes for previously identified deep intronic mutations in CEP290, OFD1, and USH2A have also been included in the GEDi probe set⁷⁹⁻³¹. The targeted regions constitute 1,210,190 bp in total (703,980 bp coding sequence), and are listed in Table 1.

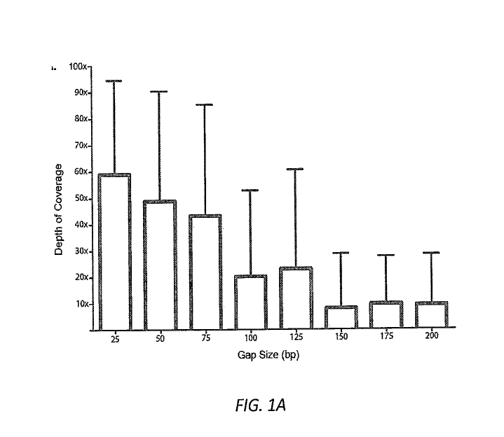

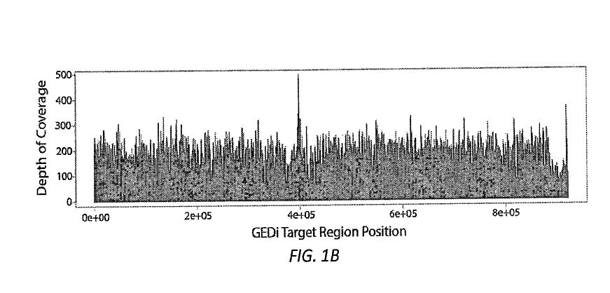

Probes for some of the targeted regions could not be designed due to the presence of repetitive or non-unique sequence elements. In total, there were 688 such design gaps ranging from 1-2,031 bp in length with an average length of 112 bp, accounting for a total of 76,980 bp (9,220 bp coding sequence). Analysis of empiric GEDi data shows that design gaps ≤75 bp (67% of gaps) were relatively well covered by “near-target” capture (FIG. 1A).

### Example 2. NGS Metrics

FIG. 1B shows a representative Depth-of-Coverage (DoC) plot for a 12×-multiplexed sample captured using the GEDi targeted enrichment kit and sequenced using an Illumina MiSeq. The data shows relatively uniform coverage of the target regions. The average percentages of the target regions covered at 1× (99.8%), 10× (98.6%) and 20× (96.4%) DoC were also relatively constant for all of the sequencing analyses. The 1.4% of target regions which were not covered with ≥10× read depth included part or all of 14 exons. The overall average DoC for all samples analyzed was 98.8×±14.5×.

### Example 3. Test Performance Metrics

The Nex-StoCT and ACMG recommend that validation of an NGS-based diagnostic test include performance test characteristics for assay accuracy, analytical sensitivity and specificity, reproducibility and repeatability^{7,8}. To measure these parameters for the GEDi capture and sequencing test, 4 samples (three randomly selected patient samples and the NA12878 HapMap sample) were prepared and sequenced in triplicate on each of three separate days. We also performed WES and SNP array genotyping analyses of these 4 samples using Agilent V4+UTR whole exome enrichment kit and Illumina Omni 2.5 SNP arrays, respectively (see Methods). The HapMap sample was included as an internal control for establishing QC metrics, and is included in all diagnostic runs to evaluate each diagnostic capture and sequencing run.

### Example 4. Sensitivity and Specificity

To assess the sensitivity and specificity of the GEDi test, we used the 2,443 SNPs located in GEDi genes that are represented on Omni 2.5 SNP array, using the Omni 2.5 data as the “gold standard.” For these analyses, sensitivity was calculated as the ability of the GEDi test to correctly identify a SNP when it was identified in the Omni 2.5 data. Similarly, the specificity was calculated as the ability of the GEDi test to correctly identify the lack of a variant at a given position when reference was detected by the Omni 2.5 array⁵ (Table 13). For example, 495±1 SNPs identified in the 9 GEDi replicates for the OGI-132-357 sample (range 492-497) were also identified in the Omni 2.5 data, and these were scored as true positives (Table 13). The GEDi test did not identify variants at 10 ±1.4 positions where variants were identified in the Omni 2.5 data for OGI-132-357, and these were scored as false negatives, giving a sensitivity of 0.98 for variant detection. The GEDi test did not identify variants at any of the 1,919 SNPs with reference genotypes in the Omni 2.5 data, for a specificity of 1 (Table 13). The average sensitivity of the GEDi test, including data from the 9 replicates of all 4 samples, was 0.979±0.007, and the specificity was 1±0.

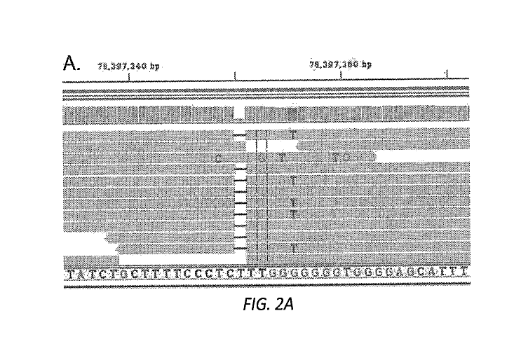

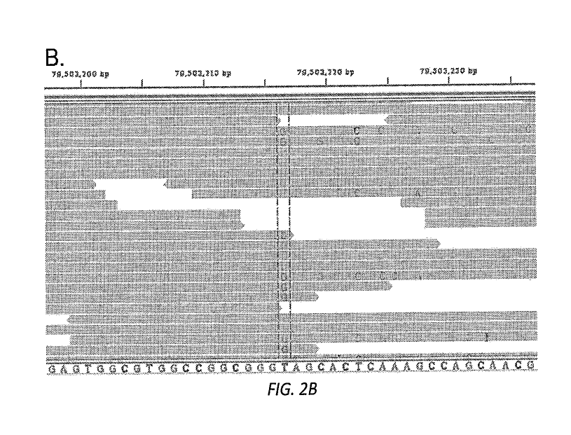

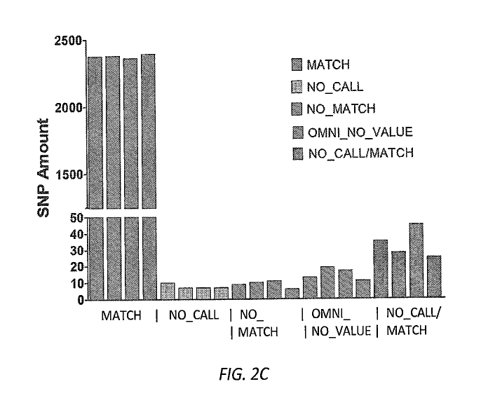

We investigated the false negative base calls in the GEDi data further and found that there were 7-11 discrepancies per sample identified between the GEDi and Omni 2.5 data (Table 14). In total, there were discrepancies detected at 23 positions that were predominantly related to the heterozygous vs. homozygous state of the same identified base, with a different base identified at only 1 position, chr15:78397352. The NGS data showed that at chr15:78397352 the Omni data were incorrect due to a single base deletion adjacent to the interrogated base, which shifted the base analyzed by the single-base extension method used in the Omni arrays (FIG. 2A). Indels were associated with 4 additional GEDi vs. Omni discrepancies, and all but one of the remaining differences were due to low SNP quality scores in the SAMtools variant identification software (Table 14). At one position (chr4:6304087), the Omni data was incorrect (confirmed by Sanger sequencing), without any evident explanation. A small number (7-10) of bases were not called in the GEDi data across all replicates (No Call, FIG. 2C), and Omni 2.5 SNP calls were not obtained for 11-19 positions (Omni No Value, FIG. 2C). Bases were scored as “No Call/Match” if 1 or more replicates for each DNA sample had no call at that position, but all other replicates matched. There were 25-45 of these bases, with many of these (55/87 total=63%) being due to no call in a single replicate (Table 2).

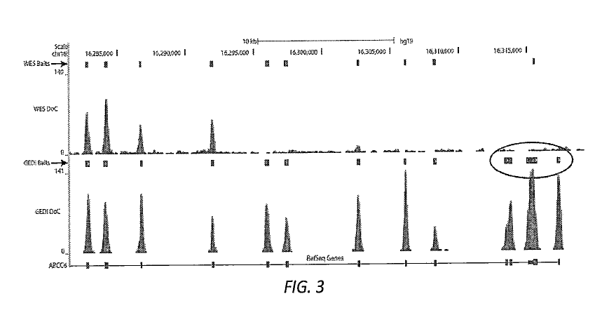

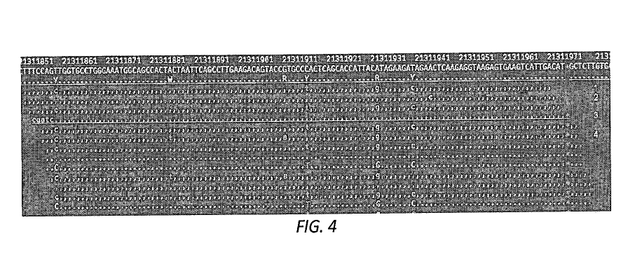

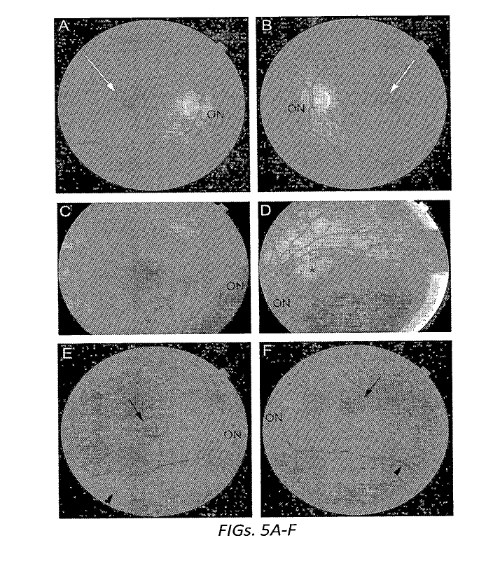

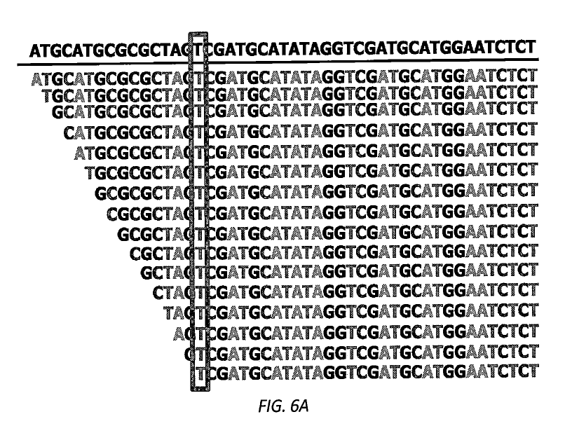

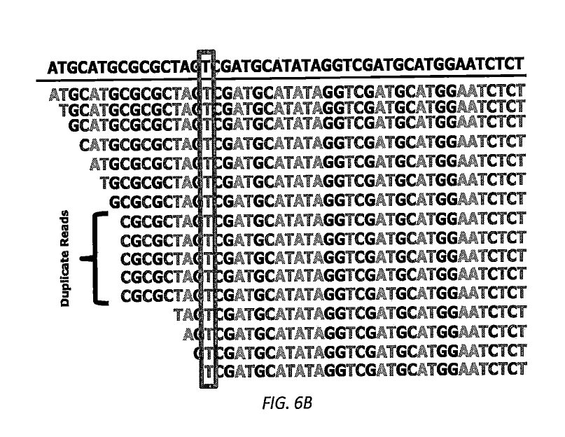

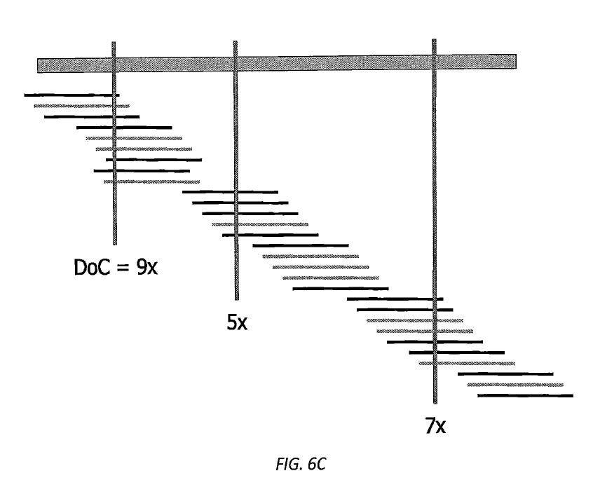

The accuracy of the GEDi test was also supported by comparison of the GEDi sequence data for the HapMap sample NA12878 with publically available Platinum 200× average depth WGS data for NA12878 from Illumina. Within the 1,197,667 bp in the GEDi capture regions, excluding the mitochondrial chromosome, there were 962 SNPs and 89 indels identified in the NA12878 WGS data by Illumina. The accuracy of the GEDi test to identify both the SNPs and indels was 99.9%. The sensitivity and specificity for SNP detection were 96.4% and 99.9%, and for indel detection were 91.6% and 99.9%, respectively. It is likely that the sensitivity of the GEDi test for SNP detection is even higher, as we identified 47 SNPs called in the Illumina Platinum data that are located in a highly repetitive 11 kb chr17 region (chr17:21311917-21323163) in the gene KCNJ12 that are likely to be incorrect due to poor read alignment (FIG. 4).

TABLE 2
Specific “No Call/Match” bases identified in GEDi vs Omni 2.5
comparison. Bases were scored as “No Call/Match” if 1 or more replicate for each
DNA sample had no call at that position, but all other replicates matched.

| CHROM | POS | NA12878 | OGI-132-357 | OGI-281-608 | OGI-307-717 |
|---|---|---|---|---|---|
| chr1 | 43638457 | 1/9 | | | |
| chr1 | 94473845 | | 3/9 | | |
| chr1 | 94486889 | 1/9 | | | |
| chr1 | 103463976 | 1/9 | | | 2/9 |
| chr1 | 156146218 | 1/9 | | | |
| chr1 | 186101539 | | 1/9 | 1/9 | |
| chr1 | 211652466 | | | 2/9 | |
| chr1 | 213071341 | 1/9 | | | |
| chr2 | 38302923 | | 2/9 | 3/9 | |
| chr2 | 74444431 | 1/9 | | 3/9 | |
| chr2 | 166770120 | | 1/9 | 4/9 | 1/9 |
| chr3 | 52804924 | 2/9 | 3/9 | 5/9 | 2/9 |
| chr3 | 193413502 | 4/9 | | | |
| chr4 | 652826 | | 1/9 | | |
| chr4 | 15482360 | 3/9 | 1/9 | | 1/9 |
| chr4 | 15981874 | 1/9 | | | |
| chr4 | 15982166 | | | | 4/9 |
| chr4 | 16020162 | | 1/9 | | |
| chr4 | 111543554 | | | 1/9 | |
| chr4 | 122748996 | 1/9 | | | |
| chr4 | 123653926 | 1/9 | | | |
| chr5 | 33954511 | 1/9 | 1/9 | 1/9 | |
| chr5 | 149323772 | 1/9 | | | |
| chr5 | 178405421 | | | | 4/9 |
| chr5 | 178407229 | | | | 1/9 |
| chr6 | 42665565 | 1/9 | | | |
| chr6 | 42932200 | 2/9 | 6/9 | 3/9 | 2/9 |
| chr6 | 42932202 | 1/9 | 2/9 | 4/9 | 3/9 |
| chr6 | 65622463 | | 1/9 | | 1/9 |
| chr6 | 70926357 | 2/9 | | 6/9 | 3/9 |
| chr6 | 72809641 | 1/9 | | | |
| chr6 | 137143748 | 3/9 | 5/9 | 6/9 | 2/9 |
| chr7 | 130037805 | 6/9 | | | |
| chr8 | 63998434 | 3/9 | 7/9 | 8/9 | 5/9 |
| chr9 | 12709480 | | | 1/9 | |
| chr9 | 117267172 | | | 2/9 | |
| chr9 | 120478032 | | | | 2/9 |
| chr9 | 139323799 | | | 3/9 | |
| chr9 | 139333236 | | | 1/9 | |
| chr10 | 13320121 | 1/9 | 1/9 | 2/9 | |
| chr10 | 55755491 | | | | 1/9 |
| chr10 | 73461805 | | | 1/9 | |
| chr10 | 73472565 | | | 1/9 | |
| chr10 | 73537449 | 2/9 | 6/9 | 4/9 | 1/9 |
| chr10 | 86004873 | | | 1/9 | |
| chr10 | 95353767 | | 1/9 | | |
| chr10 | 102505815 | 1/9 | | 5/9 | 2/9 |
| chr10 | 102588585 | | | 1/9 | |
| chr10 | 102778967 | 1/9 | 1/9 | 3/9 | |
| chr11 | 45931218 | | 1/9 | | |
| chr11 | 45935689 | 2/9 | | 1/9 | |
| chr11 | 76869378 | | | 1/9 | |
| chr11 | 76914151 | | 1/9 | 5/9 | |
| chr11 | 76917220 | 1/9 | 1/9 | | |
| chr11 | 119217254 | | | 1/9 | |
| chr12 | 88481632 | 1/9 | | | 1/9 |
| chr12 | 124417989 | | | 2/9 | |
| chr13 | 27261862 | | 1/9 | 1/9 | |
| chr14 | 74976852 | | | | 1/9 |
| chr14 | 74988687 | 3/9 | 1/9 | 5/9 | 1/9 |
| chr14 | 74992800 | | | 8/9 | 8/9 |
| chr14 | 75019002 | 2/9 | | 3/9 | 1/9 |
| chr14 | 75022271 | 1/9 | | | |
| chr14 | 75022312 | 1/9 | 1/9 | | |
| chr14 | 88852166 | | | 1/9 | |
| chr14 | 88857819 | 1/9 | | 1/9 | |
| chr15 | 73027478 | | | 1/9 | |
| chr15 | 78397352 | 1/9 | | | |
| chr16 | 1260969 | 1/9 | 1/9 | 2/9 | |
| chr16 | 1265600 | 1/9 | 2/9 | 1/9 | |
| chr16 | 16263663 | | | 1/9 | |
| chr16 | 57917149 | | | | 1/9 |
| chr16 | 57954424 | | | 1/9 | |
| chr16 | 68725783 | | 2/9 | | |
| chr17 | 7923564 | | 1/9 | | |
| chr17 | 11795157 | | 1/9 | | |
| chr17 | 11795158 | | 1/9 | | |
| chr19 | 6686192 | | 1/9 | 1/9 | |
| chr19 | 6697829 | | | | 1/9 |
| chr19 | 6709848 | | | 3/9 | |
| chr19 | 6718387 | | 1/9 | | |
| chr19 | 33167208 | | | 4/9 | |
| chr19 | 46088108 | 1/9 | | | |
| chr19 | 48344849 | | | | 1/9 |
| chr20 | 10624502 | 1/9 | | 1/9 | |
| chr22 | 33257163 | | | 7/9 | 8/9 |
| chrX | 41333549 | | 1/9 | | |

* #NO_CALL/TOTAL_REPLIC

TABLE 13
Sensitivity and specificity calculations for GEDi vs. Omni 2.5 SNP
data. For each DNA sample, the number of positions at which
variants (SNP) or reference (REF) were detected
by the Omni 2.5 SNP arrays and GEDi test are
indicated. For the GEDi data, the ranges derived from the
9 replicates for each sample tested are shown.
The average sensitivity and specificity for each DNA
sample are shown, with the ranges and standard deviation
(SD) included in parentheses. The overall sensitivity
and specificity reported in the text are the averages of these
data for all 4 samples. A standard 2 × 2 contingency table with
definitions of sensitivity and specificity is shown for reference.

| | 2 × 2 Contingency Table | |
|---|---|---|
| | Omni + (SNP) | Omni − (REF) |
| GEDi + (SNP) | True Positive | False Positive |
| GEDi − (REF) | False Negative | True Negative |
| | Sensitivity = True Pos/ | Specificity = True Neg/ |
| | (True Pos + False Neg) | (True Neg + False Pos) |
| | NA12878 | |
| | Omni + (SNP) | Omni − (REF) |
| GEDi + (SNP) | 508 (503-510 SD: 2) | 0 (0-0 SD: 0) |
| GEDi − (REF) | 12 (8-20 SD: 4) | 1933 (1919-1944 SD: 13) |
| | Sensitivity: 0.977 | Specificity: 1 (1-1 SD: 0) |
| | (0.967-0.981 SD: 0.004) | |
| | OGI-281-608 | |
| | Omni + (SNP) | Omni − (REF) |
| GEDi + (SNP) | 469 (462-472 SD: 3) | 0 (0-0 SD: 0) |
| GEDi − (REF) | 13 (10-20 SD: 3) | 1944 (1944-1944 SD: 0) |
| | Sensitivity: 0.973 | Specificity: 1 (1-1 SD: 0) |
| | (0.959-0.979 SD: 0.007) | |
| | OGI-132-357 | |
| | Omni + (SNP) | Omni − (REF) |
| GEDi + (SNP) | 495 (492-497 SD: 1) | 0 (0-0 SD:0) |
| GEDi − (REF) | 10 (8-13 SD: 1.4) | 1919 (1919-1919 SD: 0) |
| | Sensitivity: 0.981 | Specificity: 1 (1-1 SD: 0) |
| | (0.974-0.984 SD: 0.003) | |
| | OGI-307-717 | |
| | Omni + (SNP) | Omni − (REF) |
| GEDi + (SNP) | 508 (506-511 SD: 1.716) | 0 (0-0 SD:0) |
| GEDi − (REF) | 7 (4-9 SD: 2) | 1917 (1917-1917 SD: 0) |
| | Sensitivity: 0.986 | Specificity: 1 (1-1 SD: 0) |
| | (0.983-0.992 SD: 0.003) | |

TABLE 14
GEDi vs. Omni 2.5 Discrepancies Detected. Base positions of
discrepancies detected in at least 1 GEDi sequence replicate and the Omni 2.5 SNP
data for each sample analyzed are shown. The number of replicates with alternate
results are indicated. Fewer than 9 replicates are indicated for some positions at
which base calls were not made in some replicates. Discrepancies located in the top
portion of the table were due to low SNP quality scores in SAMtools; the bottom
portion of the table contains discrepancies specific to the GEDi vs. Omni data
comparisons. The reasons identified for the discrepancies are indicated in the right
column: 1 = low SNP quality score; 2 = heterozygous deletion of target base; 3 =
homozygous deletion adjacent to target base; 4 = heterozygous insertion adjacent to
target base; 5 = Omni incorrect, reason uncertain. Note that position chr4: 15982166
is included in both halves of the table.

| | NA12878 | OGI-132-357 | OGI-281-608 | OGI-307-717 | DISCREPANCY |
|---|---|---|---|---|---|
| chr1: 156146218 | | | 1/9 HOM/HET | | 1 |
| chr1: 213071341 | | | | 1/9 HOM/HET | 1 |
| chr2: 62052380 | | | 1/9 HOM/HET | | 1 |
| chr2: 166770120 | | 3/8 HOM/HET | | | 1 |
| chr3: 150645351 | 2/9 HOM/HET | | | | 1 |
| chr3: 193413502 | 1/5 HOM/HET | | | | 1 |
| chr4: 15982166 | | | | 2/7 HOM/HET | 1 |
| chr5: 178405941 | 2/9 HOM/HET | | | 1/9 HOM/HET | 1 |
| chr6: 42932200 | 1/7 HOM/HET | 1/3 HOM/HET | | 1/7 HOM/HET | 1 |
| chr6: 42932202 | 1/8 HOM/HET | 1/7 HOM/HET | | | 1 |
| chr9: 102861613 | | | 1/9 HOM/HET | | 1 |
| chr9: 139327064 | | 9/9 HOM/HET | 9/9 HOM/HET | 9/9 HOM/HET | 1 |
| chr10: 73461805 | | | 1/9 HOM/HET | | 1 |
| chr10: 85976966 | 9/9 HOM/HET | 9/9 HOM/HET | | | 1 |
| chr16: 1265600 | | 1/7 HOM/HET | | | 1 |
| chr16: 1574863 | | 1/9 HOM/HET | | | 1 |
| chr16: 57937788 | | | 1/9 HOM/HET | | 1 |
| chr17: 11835331 | | | 1/9 HOM/HET | | 1 |
| chr3: 63986047 | 9/9 HOM/HET | | 9/9 HOM/HET | | 2 |
| chr4: 6304087 | | | | 9/9 HOM/HET | 5 |
| chr4: 15981874 | | 9/9 HOM/HET | | | 4 |
| chr4: 15982166 | 9/9 HOM/HET | | 9/9 HOM/HET | | 4 |
| chr11: 76895772 | | 9/9 HOM/HET | 9/9 HOM/HET | | 2 |
| chr15: 78397352 | 9/9 DISCREP | 9/9 DISCREP | 9/9 DISCREP | 9/9 DISCREP | 3 |

### Example 5. GEDi vs. WES

The GEDi test performance was compared to WES by analyzing the WES data of the same 4 validation samples for the 2,443 Omni 2.5 SNPs in the GEDi gene set. The average depth of coverage achieved by WES in these experiments was 100×, with 98% of the targeted regions covered at 10× sequence depth. Using the Omni 2.5 data as the “gold standard,” the sensitivity of WES was 0.883±0.004, and the specificity of WES was 0.9998±0.0003. While both GEDi and WES have excellent specificity, this comparison shows that WES is approximately 10% less sensitive than the GEDi test. Analysis shows that this is due to lack of sequence coverage in the

WES data, with approximately 10% of the 2,443 positions interrogated in these analyses having insufficient coverage (≥10×) to make an accurate base call (Table 3). The majority of these positions (76%) were common in all 4 samples, suggesting that these positions were covered less efficiently in the WES capture design. Comparison of the WES and GEDi capture baits at these positions confirmed this hypothesis, and showed that 88% of the positions without coverage in the WES data had no baits in the V4+UTR capture set, whereas the GEDi capture set had at least 1 bait at these positions (Table 3). An example of one of these regions in shown in FIG. 3. Further, there are 947 mutations in IRD disease genes reported in HGMD, ClinVar and Ensemble that would be detected by GEDi sequencing which fall in regions that are not covered in the Agilent V4+UTR WES capture set (Table 4). Conversely, WES did detect bases at an average of 5.5 positions out of 2,443 (0.22%) for which GEDi sequencing provided no call.

TABLE 3
Positions at which WES did not provide sequence coverage. Base
positions at which no call was made in WES due to lack of sequence coverage are
indicated, along with the number of GEDi and WES capture baits at each position.

| | | | OGI- | OGI- | OGI- | | | |
|---|---|---|---|---|---|---|---|---|
| | | | 132- | 281- | 307- | | GEDi # | V4 + UTR # |
| CHR | POS | NA12878 | 357 | 608 | 717 | RESULT | baits | baits |
| chr1 | 20396714 | X | X | X | X | 4/4 | 1 | 0 |
| chr1 | 20396733 | X | X | X | X | 4/4 | 1 | 0 |
| chr1 | 20396902 | X | X | X | X | 4/4 | 2 | 0 |
| chr1 | 20417765 | | X | X | X | 3/4 | 2 | 0 |
| chr1 | 20418323 | X | X | X | X | 4/4 | 2 | 0 |
| chr1 | 103360951 | X | X | X | X | 4/4 | 1 | 0 |
| chr1 | 103463976 | X | X | | X | 3/4 | 1 | 1 |
| chr1 | 211665298 | X | X | X | X | 4/4 | 2 | 0 |
| chr1 | 211665299 | X | X | X | X | 4/4 | 2 | 0 |
| chr1 | 211665454 | X | X | X | X | 4/4 | 2 | 0 |
| chr1 | 211665475 | X | X | X | X | 4/4 | 2 | 0 |
| chr1 | 211666218 | X | X | X | X | 4/4 | 1 | 0 |
| chr1 | 216498911 | | X | X | X | 3/4 | 2 | 0 |
| chr2 | 27667297 | X | X | X | | 3/4 | 2 | 1 |
| chr2 | 38302923 | X | X | X | X | 4/4 | 1 | 0 |
| chr2 | 38303095 | X | X | X | X | 4/4 | 3 | 0 |
| chr2 | 74445333 | X | X | X | X | 4/4 | 2 | 0 |
| chr2 | 74445786 | X | X | X | X | 4/4 | 2 | 0 |
| chr2 | 74466594 | | X | X | X | 3/4 | 2 | 0 |
| chr2 | 74561260 | X | X | X | X | 4/4 | 2 | 0 |
| chr2 | 98962749 | X | X | X | X | 4/4 | 2 | 0 |
| chr2 | 182401388 | X | X | | X | 3/4 | 3 | 0 |
| chr2 | 202502195 | X | X | X | X | 4/4 | 1 | 0 |
| chr2 | 232597042 | X | X | X | X | 4/4 | 1 | 0 |
| chr2 | 233631062 | X | X | X | X | 4/4 | 2 | 0 |
| chr2 | 234216628 | X | X | X | X | 4/4 | 1 | 0 |
| chr2 | 234228005 | X | X | X | X | 4/4 | 1 | 0 |
| chr3 | 48507182 | | | X | | 1/4 | 2 | 0 |
| chr3 | 50233215 | X | X | X | X | 4/4 | 3 | 0 |
| chr3 | 50233317 | X | X | X | X | 4/4 | 2 | 0 |
| chr3 | 50233873 | X | X | X | X | 4/4 | 2 | 0 |
| chr3 | 50234126 | X | X | X | X | 4/4 | 3 | 0 |
| chr3 | 97483609 | X | X | X | X | 4/4 | 1 | 0 |
| chr3 | 100945367 | X | X | X | X | 4/4 | 2 | 0 |
| chr3 | 100945369 | X | X | X | X | 4/4 | 2 | 0 |
| chr3 | 132399969 | X | X | X | X | 4/4 | 2 | 0 |
| chr3 | 132399995 | X | X | X | X | 4/4 | 2 | 0 |
| chr3 | 142522855 | X | | X | X | 3/4 | 1 | 1 |
| chr3 | 142524858 | X | | | | 1/4 | 1 | 1 |
| chr3 | 193412609 | X | X | | X | 3/4 | 4 | 0 |
| chr3 | 193412672 | X | X | X | X | 4/4 | 5 | 0 |
| chr3 | 193412949 | X | X | X | X | 4/4 | 5 | 0 |
| chr3 | 193413502 | X | X | X | X | 4/4 | 4 | 0 |
| chr3 | 193413732 | X | X | X | X | 4/4 | 6 | 0 |
| chr3 | 193413814 | X | X | X | X | 4/4 | 4 | 0 |
| chr3 | 193414481 | X | X | X | X | 4/4 | 4 | 0 |
| chr3 | 193414675 | X | X | X | X | 4/4 | 4 | 0 |
| chr3 | 193414733 | X | X | X | X | 4/4 | 4 | 0 |
| chr3 | 193414780 | X | X | X | X | 4/4 | 4 | 0 |
| chr3 | 193414797 | X | X | X | X | 4/4 | 4 | 0 |
| chr4 | 15539735 | X | X | X | X | 4/4 | 2 | 0 |
| chr4 | 15970349 | X | X | X | X | 4/4 | 2 | 0 |
| chr4 | 39287322 | X | X | X | X | 4/4 | 3 | 0 |
| chr4 | 39287419 | X | X | X | X | 4/4 | 1 | 0 |
| chr4 | 111558994 | X | X | X | X | 4/4 | 2 | 0 |
| chr4 | 123653926 | X | X | X | X | 4/4 | 3 | 0 |
| chr4 | 186990344 | X | X | X | X | 4/4 | 1 | 0 |
| chr4 | 186990345 | X | X | X | X | 4/4 | 1 | 0 |
| chr4 | 187132943 | X | X | X | X | 4/4 | 2 | 0 |
| chr4 | 187133031 | X | X | X | X | 4/4 | 2 | 0 |
| chr4 | 187133433 | X | X | X | X | 4/4 | 2 | 0 |
| chr4 | 187133576 | X | X | X | X | 4/4 | 3 | 0 |
| chr4 | 187133901 | X | X | X | X | 4/4 | 2 | 0 |
| chr4 | 187134084 | X | X | X | X | 4/4 | 3 | 0 |
| chr5 | 43192421 | X | X | X | X | 4/4 | 4 | 0 |
| chr5 | 43192453 | X | X | X | X | 4/4 | 3 | 0 |
| chr5 | 90024735 | | | X | | 1/4 | 1 | 1 |
| chr5 | 90085654 | X | | | | 1/4 | 1 | 1 |
| chr5 | 178405421 | X | X | X | X | 4/4 | 2 | 0 |
| chr5 | 178405711 | X | X | X | X | 4/4 | 2 | 0 |
| chr5 | 178405729 | X | X | X | X | 4/4 | 3 | 0 |
| chr5 | 178405897 | X | X | X | X | 4/4 | 2 | 0 |
| chr5 | 178405912 | X | X | X | X | 4/4 | 1 | 0 |
| chr5 | 178405941 | X | X | X | X | 4/4 | 1 | 0 |
| chr5 | 178406299 | X | X | X | X | 4/4 | 1 | 0 |
| chr5 | 178406388 | X | X | X | X | 4/4 | 2 | 0 |
| chr5 | 178406467 | X | X | X | X | 4/4 | 2 | 0 |
| chr5 | 178407593 | | X | X | X | 3/4 | 1 | 0 |
| chr5 | 178408344 | X | X | X | X | 4/4 | 2 | 0 |
| chr6 | 31901322 | | X | X | | 2/4 | 1 | 0 |
| chr6 | 42142244 | X | X | X | X | 4/4 | 1 | 0 |
| chr6 | 42151556 | X | X | X | X | 4/4 | 3 | 0 |
| chr6 | 42151625 | X | X | X | X | 4/4 | 2 | 0 |
| chr6 | 42942583 | X | X | | | 2/4 | 1 | 1 |
| chr6 | 65622463 | | X | | | 1/4 | 2 | 2 |
| chr6 | 70962039 | | | | X | 1/4 | 1 | 1 |
| chr6 | 70962044 | | | | X | 1/4 | 1 | 1 |
| chr6 | 70981794 | X | | | | 1/4 | 1 | 1 |
| chr7 | 23145952 | | X | X | X | 3/4 | 1 | 0 |
| chr7 | 91715662 | | X | | X | 2/4 | 2 | 1 |
| chr7 | 128415623 | | X | | | 1/4 | 2 | 1 |
| chr7 | 130033630 | X | X | X | X | 4/4 | 1 | 0 |
| chr7 | 130034695 | X | X | X | X | 4/4 | 2 | 0 |
| chr8 | 77912424 | X | X | X | X | 4/4 | 3 | 0 |
| chr8 | 87755891 | X | | | X | 2/4 | 1 | 0 |
| chr8 | 94831346 | X | X | X | X | 4/4 | 4 | 0 |
| chr9 | 102861568 | X | X | X | X | 4/4 | 4 | 0 |
| chr9 | 102861613 | X | X | X | X | 4/4 | 6 | 0 |
| chr9 | 117265406 | X | X | X | X | 4/4 | 2 | 0 |
| chr9 | 120479337 | X | X | X | X | 4/4 | 3 | 0 |
| chr10 | 13142251 | X | X | X | X | 4/4 | 2 | 0 |
| chr10 | 13150265 | X | X | X | X | 4/4 | 1 | 0 |
| chr10 | 50747071 | X | X | X | X | 4/4 | 4 | 0 |
| chr10 | 73157051 | | X | X | | 2/4 | 2 | 1 |
| chr10 | 86004865 | | | X | | 1/4 | 3 | 1 |
| chr10 | 86004873 | | | X | X | 2/4 | 2 | 1 |
| chr10 | 102505815 | | | X | | 1/4 | 1 | 2 |
| chr10 | 102778967 | X | X | X | X | 4/4 | 2 | 0 |
| chr10 | 124249118 | | X | | | 1/4 | 1 | 0 |
| chr10 | 126089434 | X | X | | X | 3/4 | 2 | 0 |
| chr11 | 12308462 | X | X | X | X | 4/4 | 1 | 0 |
| chr11 | 12308485 | X | X | X | X | 4/4 | 1 | 0 |
| chr11 | 12308500 | X | X | X | X | 4/4 | 2 | 0 |
| chr11 | 45931502 | | | X | | 1/4 | 4 | 2 |
| chr11 | 61717607 | X | X | X | X | 4/4 | 2 | 0 |
| chr11 | 67227006 | X | X | X | X | 4/4 | 2 | 0 |
| chr11 | 76833819 | | X | | | 1/4 | 1 | 0 |
| chr11 | 76853829 | | | X | | 1/4 | 2 | 0 |
| chr11 | 76883800 | X | X | X | | 3/4 | 2 | 0 |
| chr11 | 76895772 | X | | | | 1/4 | 4 | 2 |
| chr11 | 76914151 | X | X | X | X | 4/4 | 4 | 1 |
| chr11 | 117198609 | X | X | X | X | 4/4 | 2 | 0 |
| chr11 | 117252437 | | X | X | X | 3/4 | 2 | 0 |
| chr11 | 117261853 | | | X | | 1/4 | 3 | 1 |
| chr12 | 1901546 | X | X | X | X | 4/4 | 1 | 0 |
| chr12 | 1902025 | X | X | X | X | 4/4 | 2 | 0 |
| chr12 | 1902164 | X | X | X | X | 4/4 | 2 | 0 |
| chr12 | 1902354 | X | X | X | X | 4/4 | 2 | 0 |
| chr12 | 1920835 | X | X | X | X | 4/4 | 2 | 1 |
| chr12 | 1920853 | X | X | X | | 3/4 | 2 | 1 |
| chr12 | 1982754 | X | X | X | X | 4/4 | 1 | 0 |
| chr12 | 7362960 | X | X | X | | 3/4 | 2 | 0 |
| chr12 | 7363447 | X | X | X | X | 4/4 | 2 | 0 |
| chr12 | 7363916 | X | X | X | X | 4/4 | 2 | 0 |
| chr12 | 15125989 | X | X | x | X | 4/4 | 2 | 0 |
| chr12 | 15134711 | X | | | | 1/4 | 2 | 0 |
| chr12 | 15134729 | X | X | X | X | 4/4 | 2 | 0 |
| chr12 | 48371802 | X | | | | 1/4 | 2 | 0 |
| chr12 | 56114181 | X | X | X | X | 4/4 | 3 | 0 |
| chr12 | 76738345 | X | X | | X | 3/4 | 1 | 1 |
| chr13 | 48828118 | | | | X | 1/4 | 1 | 0 |
| chr14 | 68189208 | X | X | X | X | 4/4 | 3 | 0 |
| chr14 | 74965434 | X | X | X | X | 4/4 | 2 | 0 |
| chr14 | 74965543 | X | X | X | X | 4/4 | 2 | 0 |
| chr14 | 74966763 | X | X | X | X | 4/4 | 2 | 0 |
| chr14 | 74969615 | | | X | X | 2/4 | 1 | 0 |
| chr14 | 88857819 | X | | | | 1/4 | 1 | 1 |
| chr14 | 92336054 | X | X | X | X | 4/4 | 2 | 0 |
| chr14 | 92336239 | X | X | X | X | 4/4 | 2 | 0 |
| chr14 | 92336242 | X | X | X | X | 4/4 | 2 | 0 |
| chr14 | 92409333 | | | | X | 1/4 | 1 | 1 |
| chr15 | 31453147 | X | X | X | X | 4/4 | 3 | 0 |
| chr15 | 65914282 | X | X | X | X | 4/4 | 1 | 0 |
| chr15 | 65916306 | X | X | X | | 3/4 | 1 | 0 |
| chr15 | 65948035 | X | X | X | X | 4/4 | 2 | 0 |
| chr15 | 65948348 | X | X | X | X | 4/4 | 2 | 0 |
| chr15 | 72107221 | X | X | X | X | 4/4 | 1 | 0 |
| chr15 | 89753136 | X | X | X | X | 4/4 | 1 | 0 |
| chr15 | 89753160 | X | X | X | X | 4/4 | 2 | 0 |
| chr15 | 89753194 | X | X | X | X | 4/4 | 2 | 0 |
| chr15 | 89753220 | X | X | X | X | 4/4 | 2 | 0 |
| chr15 | 89753274 | X | X | X | X | 4/4 | 3 | 0 |
| chr15 | 89764896 | X | X | X | X | 4/4 | 1 | 0 |
| chr16 | 1271240 | X | X | X | X | 4/4 | 3 | 0 |
| chr16 | 1271348 | X | X | X | X | 4/4 | 2 | 0 |
| chr16 | 1271471 | X | X | X | X | 4/4 | 2 | 0 |
| chr16 | 16255205 | X | X | X | X | 4/4 | 1 | 0 |
| chr16 | 16255240 | | X | X | X | 3/4 | 2 | 0 |
| chr16 | 16281007 | | X | X | X | 3/4 | 2 | 1 |
| chr16 | 53634295 | X | X | X | X | 4/4 | 2 | 0 |
| chr16 | 53635251 | X | X | X | X | 4/4 | 2 | 0 |
| chr16 | 56518357 | X | X | X | X | 4/4 | 2 | 0 |
| chr16 | 57916776 | X | X | X | X | 4/4 | 2 | 0 |
| chr16 | 57916835 | X | X | X | X | 4/4 | 3 | 0 |
| chr16 | 57917012 | X | X | X | X | 4/4 | 2 | 0 |
| chr16 | 57917149 | X | X | X | X | 4/4 | 1 | 0 |
| chr16 | 57949224 | X | X | | | 2/4 | 2 | 1 |
| chr16 | 57988797 | X | X | X | X | 4/4 | 1 | 0 |
| chr16 | 57991317 | | | X | | 1/4 | 1 | 1 |
| chr16 | 58000163 | X | X | X | X | 4/4 | 1 | 0 |
| chr16 | 68725783 | | X | | | 1/4 | 2 | 0 |
| chr16 | 75571923 | X | X | X | X | 4/4 | 1 | 0 |
| chr16 | 75572012 | X | X | X | X | 4/4 | 1 | 0 |
| chr16 | 75572495 | X | X | X | X | 4/4 | 2 | 0 |
| chr16 | 75573837 | X | X | X | X | 4/4 | 2 | 0 |
| chr17 | 1588141 | X | X | X | X | 4/4 | 2 | 0 |
| chr17 | 6377804 | | | X | X | 2/4 | 4 | 1 |
| chr17 | 7919931 | | | X | | 1/4 | 1 | 0 |
| chr17 | 7923564 | X | X | X | X | 4/4 | 3 | 0 |
| chr17 | 11786291 | X | X | X | X | 4/4 | 1 | 0 |
| chr17 | 11786308 | X | X | X | X | 4/4 | 1 | 0 |
| chr17 | 11786412 | X | X | X | X | 4/4 | 2 | 0 |
| chr17 | 21321281 | X | X | X | X | 4/4 | 2 | 0 |
| chr17 | 21321657 | X | X | X | X | 4/4 | 2 | 0 |
| chr17 | 21321955 | X | X | X | X | 4/4 | 2 | 0 |
| chr17 | 21322821 | X | X | X | X | 4/4 | 3 | 0 |
| chr17 | 56282763 | | | X | | 1/4 | 2 | 0 |
| chr17 | 63223643 | X | X | X | | 3/4 | 2 | 0 |
| chr17 | 63223645 | X | X | X | | 3/4 | 2 | 0 |
| chr17 | 63223684 | X | X | X | | 3/4 | 2 | 0 |
| chr17 | 63223788 | X | X | X | X | 4/4 | 1 | 0 |
| chr17 | 72912509 | X | X | X | X | 4/4 | 4 | 0 |
| chr17 | 72913320 | X | X | X | X | 4/4 | 2 | 0 |
| chr17 | 74523791 | X | X | X | X | 4/4 | 4 | 0 |
| chr17 | 79617534 | X | X | X | X | 4/4 | 2 | 0 |
| chr19 | 3769470 | X | X | X | X | 4/4 | 4 | 0 |
| chr19 | 3769753 | X | X | X | X | 4/4 | 3 | 0 |
| chr19 | 3769834 | X | X | X | X | 4/4 | 3 | 0 |
| chr19 | 3769939 | X | X | X | X | 4/4 | 1 | 0 |
| chr19 | 3770238 | X | X | X | X | 4/4 | 4 | 0 |
| chr19 | 6710683 | | | X | | 1/4 | 2 | 1 |
| chr19 | 14203542 | X | X | X | X | 4/4 | 4 | 0 |
| chr19 | 33166408 | X | X | X | X | 4/4 | 4 | 0 |
| chr19 | 33168082 | | X | X | | 2/4 | 2 | 0 |
| chr19 | 33168383 | X | X | X | X | 4/4 | 2 | 0 |
| chr19 | 33168412 | X | X | X | X | 4/4 | 2 | 0 |
| chr19 | 33168447 | X | X | X | X | 4/4 | 2 | 0 |
| chr19 | 33168605 | X | X | X | X | 4/4 | 2 | 0 |
| chr19 | 33168627 | X | X | X | X | 4/4 | 3 | 0 |
| chr19 | 33168641 | X | X | X | X | 4/4 | 2 | 0 |
| chr19 | 33168829 | X | X | X | X | 4/4 | 2 | 0 |
| chr19 | 33169003 | X | X | X | X | 4/4 | 2 | 0 |
| chr19 | 33169097 | X | X | X | X | 4/4 | 4 | 0 |
| chr19 | 46031606 | X | X | X | X | 4/4 | 3 | 0 |
| chr19 | 46032124 | X | X | X | X | 4/4 | 4 | 0 |
| chr19 | 46049687 | X | X | X | X | 4/4 | 2 | 0 |
| chr19 | 46052454 | X | X | X | X | 4/4 | 3 | 0 |
| chr19 | 46052639 | X | X | X | X | 4/4 | 1 | 0 |
| chr19 | 46053050 | X | X | X | X | 4/4 | 1 | 0 |
| chr19 | 46053550 | X | X | X | X | 4/4 | 2 | 0 |
| chr19 | 46053560 | X | X | X | X | 4/4 | 3 | 0 |
| chr19 | 46053664 | X | X | X | X | 4/4 | 3 | 0 |
| chr19 | 46053734 | X | X | X | X | 4/4 | 2 | 0 |
| chr19 | 46055385 | X | X | X | X | 4/4 | 2 | 0 |
| chr19 | 46056500 | | X | X | X | 3/4 | 2 | 0 |
| chr19 | 46088108 | | | X | | 1/4 | 1 | 0 |
| chr19 | 48325187 | X | X | X | X | 4/4 | 4 | 0 |
| chr19 | 48344270 | X | X | X | X | 4/4 | 1 | 0 |
| chr19 | 48344346 | X | X | X | X | 4/4 | 2 | 0 |
| chr19 | 48344444 | X | X | X | X | 4/4 | 2 | 0 |
| chr19 | 48344513 | X | X | X | X | 4/4 | 2 | 0 |
| chr19 | 48344570 | X | X | X | X | 4/4 | 2 | 0 |
| chr19 | 48344849 | X | X | X | X | 4/4 | 1 | 0 |
| chr19 | 48345016 | X | X | X | X | 4/4 | 2 | 0 |
| chr19 | 48345395 | X | X | X | X | 4/4 | 2 | 0 |
| chr19 | 48345407 | X | X | X | X | 4/4 | 2 | 0 |
| chr19 | 48345928 | X | X | X | X | 4/4 | 2 | 0 |
| chr19 | 48345982 | X | X | X | X | 4/4 | 3 | 0 |
| chr19 | 48346161 | X | X | X | X | 4/4 | 2 | 0 |
| chr19 | 48346241 | X | X | X | X | 4/4 | 3 | 0 |
| chr19 | 48346341 | X | X | X | X | 4/4 | 2 | 0 |
| chr19 | 48346462 | X | X | X | X | 4/4 | 3 | 0 |
| chr19 | 48346503 | X | X | X | X | 4/4 | 2 | 0 |
| chr19 | 48346525 | X | X | X | X | 4/4 | 1 | 0 |
| chr19 | 54631797 | X | X | X | X | 4/4 | 1 | 0 |
| chr20 | 3869610 | X | X | X | X | 4/4 | 1 | 0 |
| chr20 | 25282967 | X | | X | X | 3/4 | 2 | 1 |
| chr22 | 33197074 | X | X | X | X | 4/4 | 3 | 0 |
| chrX | 18665342 | | | | X | 1/4 | 2 | 0 |
| chrX | 38128538 | X | X | | X | 3/4 | 5 | 0 |
| chrX | 153421844 | X | X | X | X | 4/4 | 2 | 0 |
| chrX | 153421847 | X | X | X | X | 4/4 | 2 | 0 |
| chrX | 153776107 | X | X | X | X | 4/4 | 2 | 0 |

| | # Hits | % Hits |
|---|---|---|
| 4/4 | 199 | 75.67% |
| 3/4 | 25 | 9.51% |
| 2/4 | 10 | 3.80% |
| 1/4 | 29 | 11.03% |
| Total: | 263 | |

TABLE 4
List of mutations in IRD disease genes reported in HGMD, ClinVar
and Ensemble that would be detected by GEDi sequencing that are not covered in the
Agilent V4 + UTR WES capture set.

| Chromo- | | | | |
|---|---|---|---|---|
| some | Genomic coordinate | Gene | dbSNP | Sources |
| 1 | g.10032132G > A | NMNAT1 | | HGMD, |
| 1 | g.10035695T > C | NMNAT1 | | HGMD, |
| 1 | g.10041108T > G | NMNAT1 | | HGMD, |
| 1 | g.10042629G > T | NMNAT1 | rs368062092 | ClinVar, |
| 1 | g.10042635C > T | NMNAT1 | | HGMD, |
| 1 | g.10042642del | NMNAT1 | | HGMD, |
| 1 | g.10042671C > A | NMNAT1 | | HGMD, |
| 1 | g.12061468A > G | MFN2 | rs119103264 | ClinVar, Ensembl, |
| 1 | g.12061480G > A | MFN2 | rs28940294 | ClinVar, |
| 1 | g.12064892G > A | MFN2 | rs138382758 | ClinVar, |
| 1 | g.12071567G > C | MFN2 | rs28940292 | ClinVar, |
| 1 | g.150316677A > C | PRPF3 | rs121434243 | HGMD, |
| 1 | g.150316677C > A | PRPF3 | rs121434243 | ClinVar, Ensembl, |
| 1 | g.171605813G > A | MYOC | | HGMD, |
| 1 | g.171605826C > T | MYOC | rs74315341 | ClinVar, Ensembl, |
| 1 | g.171605828A > G | MYOC | | HGMD, |
| 1 | g.171605844C > T | MYOC | | HGMD, |
| 1 | g.171605846C > T | MYOC | rs74315340 | ClinVar, Ensembl, |
| 1 | g.171605849C > A | MYOC | | HGMD, |
| 1 | g.171605849C > G | MYOC | | HGMD, |
| 1 | g.196659324T > A | CFH | rs121913056 | ClinVar, |
| 1 | g.196716375C > T | CFH | rs121913059 | ClinVar, Ensembl, |
| 1 | g.197297561T > G | CRB1 | | HGMD, |
| 1 | g.197297588G > C | CRB1 | | HGMD, |
| 1 | g.197297593del | CRB1 | | HGMD, |
| 1 | g.197398718A > G | CRB1 | | HGMD, |
| 1 | g.197403836A > G | CRB1 | rs62645748 | HGMD, |
| 1 | g.197403836G > A | CRB1 | rs62645748 | ClinVar, Ensembl, |
| 1 | g.197403868A > G | CRB1 | | HGMD, |
| 1 | g.211654461C > T | RD3 | rs386834260 | ClinVar, HGMD, |
| 1 | g.215813957G > A | USH2A | rs397517994 | Ensembl, |
| 1 | g.215813957T > C | USH2A | | HGMD, |
| 1 | g.215820868del | USH2A | | HGMD, |
| 1 | g.215823990A > G | USH2A | | HGMD, |
| 1 | g.215823990C > T | USH2A | rs397517990 | Ensembl, |
| 1 | g.215823992G > A | USH2A | | HGMD, |
| 1 | g.215844615T > C | USH2A | | HGMD, |
| 1 | g.215844625T > C | USH2A | | HGMD, |
| 1 | g.215916678C > G | USH2A | | HGMD, |
| 1 | g.215955400A > G | USH2A | rs111033265 | HGMD, |
| 1 | g.215955400C > T | USH2A | rs111033265 | Ensembl, |
| 1 | g.215960153G > T | USH2A | | HGMD, |
| 1 | g.216052107T > A | USH2A | | HGMD, |
| 1 | g.216061779A > G | USH2A | | HGMD, |
| 1 | g.216061803G > A | USH2A | | GEDi, |
| 1 | g.216064540T > C | USH2A | | HGMD, |
| 1 | g.216166497C > A | USH2A | | GEDi, |
| 1 | g.216243443C > A | USH2A | | HGMD, |
| 1 | g.216246230C > G | USH2A | | HGMD, |
| 1 | g.216251475T > C | USH2A | | HGMD, |
| 1 | g.216251484T > G | USH2A | | HGMD, |
| 1 | g.216251489A > T | USH2A | | HGMD, |
| 1 | g.216256797C > G | USH2A | | HGMD, |
| 1 | g.216424258del | USH2A | | HGMD, |
| 1 | g.216424275C > G | USH2A | rs696723 | Ensembl, HGMD, |
| 1 | g.216498883A > C | USH2A | | HGMD, |
| 1 | g.216498883T > C | USH2A | | HGMD, |
| 1 | g.216498885T > G | USH2A | | HGMD, |
| 1 | g.216498940A > G | USH2A | | HGMD, |
| 1 | g.216591855C > T | USH2A | | HGMD, |
| 1 | g.2337267A > G | PEX10 | | HGMD, |
| 1 | g.243471334G > T | SDCCAG8 | | HGMD, |
| 1 | g.40544236G > A | PPT1 | | HGMD, |
| 1 | g.40555081C > T | PPT1 | rs386833651 | ClinVar, |
| 1 | g.40555089G > C | PPT1 | rs386833650 | ClinVar, |
| 1 | g.40562786C > T | PPT1 | rs386833628 | ClinVar, |
| 1 | g.40562794A > T | PPT1 | rs386833627 | ClinVar, |
| 1 | g.40562797C > A | PPT1 | rs386833626 | ClinVar, |
| 1 | g.40562797C > T | PPT1 | rs386833626 | ClinVar, |
| 1 | g.40562797del | PPT1 | rs386833625 | ClinVar, |
| 1 | g.5927091del | NPHP4 | | HGMD, |
| 1 | g.5950967del | NPHP4 | | HGMD, |
| 1 | g.5964848G > A | NPHP4 | rs137852923 | ClinVar, |
| 1 | g.6021854C > T | NPHP4 | | HGMD, |
| 1 | g.6021886del | NPHP4 | | HGMD, |
| 1 | g.68903976A > G | RPE65 | rs61752909 | GEDi, ClinVar, Ensembl, |
| 1 | g.68903976C > T | RPE65 | rs61752909 | HGMD, |
| 1 | g.68904631_68904632insCCA | RPE65 | | HGMD, |
| 1 | g.68904634A > G | RPE65 | rs61752908 | HGMD, |
| 1 | g.68910529C > G | RPE65 | rs61752874 | HGMD, |
| 1 | g.68910541G > A | RPE65 | rs61752871 | ClinVar, Ensembl, |
| 1 | g.68910541T > C | RPE65 | rs61752871 | HGMD, |
| 1 | g.68910558A > G | RPE65 | rs61752870 | HGMD, |
| 1 | g.68912500del | RPE65 | | HGMD, |
| 1 | g.68912507A > G | RPE65 | rs61751282 | HGMD, |
| 1 | g.68912520A > G | RPE65 | rs61751281 | HGMD, |
| 1 | g.68912543T > G | RPE65 | rs61751278 | HGMD, |
| 1 | g.94458799C > T | ABCA4 | | HGMD, |
| 1 | g.94463416C > T | ABCA4 | | HGMD, |
| 1 | g.94463425G > C | ABCA4 | rs61748521 | Ensembl, HGMD, |
| 1 | g.94463428G > A | ABCA4 | | HGMD, |
| 1 | g.94463434T > C | ABCA4 | | HGMD, |
| 1 | g.94463437T > G | ABCA4 | | HGMD, |
| 1 | g.94463460A > G | ABCA4 | | HGMD, |
| 1 | g.94466625G > A | ABCA4 | rs2297669 | Ensembl, |
| 1 | g.94466625G > T | ABCA4 | rs2297669 | Ensembl, |
| 1 | g.94466628G > A | ABCA4 | | HGMD, |
| 1 | g.94466638G > T | ABCA4 | | HGMD, |
| 1 | g.94466644del | ABCA4 | | HGMD, |
| 1 | g.94466658C > T | ABCA4 | | HGMD, |
| 1 | g.94471004A > T | ABCA4 | | HGMD, |
| 1 | g.94471022C > T | ABCA4 | | HGMD, |
| 1 | g.94471026G > A | ABCA4 | | HGMD, |
| 1 | g.94473189C > A | ABCA4 | | HGMD, |
| 1 | g.94473189C > T | ABCA4 | | HGMD, |
| 1 | g.94476413C > T | ABCA4 | | HGMD, |
| 1 | g.94476417C > T | ABCA4 | | HGMD, |
| 1 | g.94476419A > T | ABCA4 | | HGMD, |
| 1 | g.94476424 C > T | ABCA4 | | HGMD, |
| 1 | g.94476426T > C | ABCA4 | | HGMD, |
| 1 | g.94476428G > A | ABCA4 | | HGMD, |
| 1 | g.94476467T > A | ABCA4 | | HGMD, |
| 1 | g.94476486C > T | ABCA4 | | HGMD, |
| 1 | g.94480222G > C | ABCA4 | | HGMD, |
| 1 | g.94480222G > T | ABCA4 | | HGMD, |
| 1 | g.94480224A > G | ABCA4 | | HGMD, |
| 1 | g.94480232G > A | ABCA4 | | HGMD, |
| 1 | g.94480235A > T | ABCA4 | | HGMD, |
| 1 | g.94480241A > C | ABCA4 | | HGMD, |
| 1 | g.94480243C > T | ABCA4 | | HGMD, |
| 1 | g.94486815A > C | ABCA4 | rs61753018 | HGMD, |
| 1 | g.94486836G > A | ABCA4 | | HGMD, |
| 1 | g.94495000C > A | ABCA4 | | HGMD, |
| 1 | g.94495003del | ABCA4 | | HGMD, |
| 1 | g.94495005A > C | ABCA4 | | HGMD, |
| 1 | g.94495018C > A | ABCA4 | | HGMD, |
| 1 | g.94495021C > A | ABCA4 | | HGMD, |
| 1 | g.94495021C > T | ABCA4 | | HGMD, |
| 1 | g.94495034G > T | ABCA4 | | HGMD, |
| 1 | g.94495983C > T | ABCA4 | | HGMD, |
| 1 | g.94495990C > T | ABCA4 | | HGMD, |
| 1 | g.94496557G > T | ABCA4 | | HGMD, |
| 1 | g.94496562T > G | ABCA4 | | HGMD, |
| 1 | g.94496571G > A | ABCA4 | | HGMD, |
| 1 | g.94496583A > G | ABCA4 | | HGMD, |
| 1 | g.94497333C > T | ABCA4 | | HGMD, |
| 1 | g.94497360G > A | ABCA4 | | HGMD, |
| 1 | g.94502702T > C | ABCA4 | | HGMD, |
| 1 | g.94502706C > A | ABCA4 | | HGMD, |
| 1 | g.94506902G > A | ABCA4 | | HGMD, |
| 1 | g.94506907C > T | ABCA4 | | HGMD, |
| 1 | g.94506917T > G | ABCA4 | | HGMD, |
| 1 | g.94506921C > G | ABCA4 | | HGMD, |
| 1 | g.94506923C > T | ABCA4 | rs61751399 | ClinVar, Ensembl, |
| 1 | g.94506937G > A | ABCA4 | | HGMD, |
| 1 | g.94506945del | ABCA4 | | HGMD, |
| 1 | g.94506952G > T | ABCA4 | | HGMD, |
| 1 | g.94508895A > G | ABCA4 | | HGMD, |
| 1 | g.94508919G > A | ABCA4 | | HGMD, |
| 1 | g.94508934C > T | ABCA4 | | HGMD, |
| 1 | g.94512478G > T | ABCA4 | | HGMD, |
| 1 | g.94512481G > T | ABCA4 | | HGMD, |
| 1 | g.94512485T > G | ABCA4 | | HGMD, |
| 1 | g.94514513C > T | ABCA4 | | HGMD, |
| 1 | g.94520801C > T | ABCA4 | | HGMD, |
| 1 | g.94520846del | ABCA4 | | HGMD, |
| 1 | g.94520864A > G | ABCA4 | | HGMD, |
| 1 | g.94520869G > C | ABCA4 | | HGMD, |
| 1 | g.94522156C > T | ABCA4 | | HGMD, |
| 1 | g.94522194C > T | ABCA4 | | HGMD, |
| 1 | g.94522202G > T | ABCA4 | | HGMD, |
| 1 | g.94526092C > G | ABCA4 | | HGMD, |
| 1 | g.94526106G > A | ABCA4 | | HGMD, |
| 1 | g.94528137C > T | ABCA4 | | HGMD, |
| 1 | g.94528143C > T | ABCA4 | rs61749417 | Ensembl, |
| 1 | g.94528148C > G | ABCA4 | | HGMD, |
| 1 | g.94528153G > T | ABCA4 | | HGMD, |
| 1 | g.94528806A > G | ABCA4 | rs61751392 | ClinVar, Ensembl, |
| 1 | g.94528815A > C | ABCA4 | | HGMD, |
| 1 | g.94528815T > C | ABCA4 | | HGMD, |
| 1 | g.94528819G > A | ABCA4 | | HGMD, |
| 1 | g.94528836T > C | ABCA4 | | HGMD, |
| 1 | g.94528842T > C | ABCA4 | | HGMD, |
| 1 | g.94528854A > C | ABCA4 | | HGMD, |
| 1 | g.94528859A > C | ABCA4 | | HGMD, |
| 1 | g.94528874C > T | ABCA4 | | HGMD, |
| 1 | g.94543246C > T | ABCA4 | | HGMD, |
| 1 | g.94543269G > A | ABCA4 | | HGMD, |
| 1 | g.94543272del | ABCA4 | | HGMD, |
| 1 | g.94543278T > C | ABCA4 | rs138157885 | HGMD, |
| 1 | g.94543281T > G | ABCA4 | | HGMD, |
| 1 | g.94543290T > G | ABCA4 | | GEDi, |
| 1 | g.94546234G > T | ABCA4 | | HGMD, |
| 1 | g.94546248del | ABCA4 | | HGMD, |
| 1 | g.94546262G > C | ABCA4 | | HGMD, |
| 1 | g.94546265G > A | ABCA4 | | HGMD, |
| 1 | g.94564484G > A | ABCA4 | rs61750200 | ClinVar, Ensembl, |
| 1 | g.94564500G > A | ABCA4 | rs61748536 | Ensembl, |
| 1 | g.94564500G > C | ABCA4 | rs61748536 | Ensembl, HGMD, |
| 1 | g.94564508A > G | ABCA4 | | HGMD, |
| 1 | g.94564544A > G | ABCA4 | rs61748535 | HGMD, |
| 1 | g.94568571C > G | ABCA4 | | HGMD, |
| 1 | g.94568577del | ABCA4 | | HGMD, |
| 1 | g.94568581A > G | ABCA4 | rs202198282 | HGMD, |
| 2 | g.110889347del | NPHP1 | | HGMD, |
| 2 | g.112656371C > G | MERTK | | HGMD, |
| 2 | g.112765985G > C | MERTK | | HGMD, |
| 2 | g.112777100G > T | MERTK | | HGMD, |
| 2 | g.112779003T > C | MERTK | | HGMD, |
| 2 | g.112779023del | MERTK | | HGMD, |
| 2 | g.170349410G > A | BBS5 | rs179363897 | ClinVar, Ensembl, |
| 2 | g.182423333del | CERKL | rs398122964 | ClinVar, HGMD, |
| 2 | g.182521495C > T | CERKL | | HGMD, |
| 2 | g.202493952C > T | TMEM237 | | HGMD, |
| 2 | g.202494451C > A | TMEM237 | | HGMD, |
| 2 | g.234229468T > C | SAG | rs137886124 | HGMD, |
| 2 | g.234243717G > T | SAG | rs397514682 | ClinVar, |
| 2 | g.234243727del | SAG | | HGMD, |
| 2 | g.62066572G > A | FAM161A | rs202193201 | GEDi, ClinVar, Ensembl, |
| 2 | g.62066572G > C | FAM161A | rs202193201 | ClinVar, Ensembl, HGMD, |
| 2 | g.62066572T > C | FAM161A | rs202193201 | HGMD, |
| 2 | g.96962682C > T | SNRNP200 | | HGMD, |
| 2 | g.99008421C > T | CNGA3 | | HGMD, |
| 2 | g.99008427C > G | CNGA3 | | HGMD, |
| 3 | g.100949961T > C | IMPG2 | rs199867882 | HGMD, |
| 3 | g.101023121A > G | IMPG2 | rs201893645 | Ensembl, |
| 3 | g.101023121C > T | IMPG2 | rs201893545 | HGMD, |
| 3 | g.121491530A > G | IQCB1 | rs140630401 | HGMD, |
| 3 | g.121500619G > A | IQCB1 | rs121918244 | ClinVar, |
| 3 | g.129249848A > C | RHO | rs104893793 | HGMD, |
| 3 | g.129249848C > A | RHO | rs104893793 | ClinVar, Ensembl, |
| 3 | g.129249848T > C | RHO | | HGMD, |
| 3 | g.129249856C > T | RHO | | HGMD, |
| 3 | g.129249858G > C | RHO | | HGMD, |
| 3 | g.129249862C > G | RHO | | HGMD, |
| 3 | g.129249866G > C | RHO | | HGMD, |
| 3 | g.129249869A > C | RHO | | HGMD, |
| 3 | g.129249869T > C | RHO | | HGMD, |
| 3 | g.129249877A > G | RHO | | HGMD, |
| 3 | g.129249884T > C | RHO | | HGMD, |
| 3 | g.129251096A > G | RHO | rs104893776 | ClinVar, Ensembl, |
| 3 | g.129251096G > A | RHO | rs104893776 | HGMD, |
| 3 | g.129251098T > A | RHO | | HGMD, |
| 3 | g.129251101G > C | RHO | | HGMD, |
| 3 | g.129251101T > C | RHO | | HGMD, |
| 3 | g.129251104A > G | RHO | | HGMD, |
| 3 | g.129251107A > G | RHO | rs104893780 | HGMD, |
| 3 | g.129251107G > A | RHO | rs104893780 | ClinVar, Ensembl, |
| 3 | g.129252450G > A | RHO | | HGMD, |
| 3 | g.129252450G > T | RHO | | HGMD, |
| 3 | g.129252456dup | RHO | | HGMD, |
| 3 | g.129252459G > C | RHO | | HGMD, |
| 3 | g.129252473A > C | RHO | rs138831590 | HGMD, |
| 3 | g.129252494del | RHO | | HGMD, |
| 3 | g.132432009C > G | NPHP3 | rs119456960 | ClinVar, |
| 3 | g.193311167T > A | OPA1 | | HGMD, |
| 3 | g.193311169T > G | OPA1 | | HGMD, |
| 3 | g.193311172A > G | OPA1 | | HGMD, |
| 3 | g.193311188T > G | OPA1 | | HGMD, |
| 3 | g.193354983G > A | OPA1 | | HGMD, |
| 3 | g.193355740G > A | OPA1 | | HGMD, |
| 3 | g.193355740G > T | OPA1 | | HGMD, |
| 3 | g.193361414G > A | OPA1 | | HGMD, |
| 3 | g.50230572G > A | GNAT1 | rs104893740 | ClinVar, Ensembl, |
| 4 | g.100530134G > T | MTTP | rs199422222 | ClinVar, |
| 4 | g.111543456A > T | PITX2 | rs28936408 | Ensembl, |
| 4 | g.13378177T > C | RAB28 | | HGMD, |
| 4 | g.13383201G > A | RAB28 | rs398123044 | ClinVar, |
| 4 | g.13383201T > C | RAB28 | | HGMD, |
| 4 | g.15542617C > T | CC2D2A | | HGMD, |
| 4 | g.15565097T > C | CC2D2A | | HGMD, |
| 4 | g.15565108C > G | CC2D2A | rs386833750 | ClinVar, |
| 4 | g.15565108C > T | CC2D2A | rs386833750 | ClinVar, |
| 4 | g.15569105G > C | CC2D2A | | HGMD, |
| 4 | g.16010716A > T | PROM1 | | GEDi, |
| 4 | g.16010732C > T | PROM1 | | HGMD, |
| 4 | g.187117161C > T | CYP4V2 | rs119103283 | HGMD, |
| 4 | g.187117161T > C | CYP4V2 | rs119103283 | ClinVar, Ensembl, |
| 4 | g.187117164G > T | CYP4V2 | rs199476188 | HGMD, |
| 4 | g.187117164T > G | CYP4V2 | rs199476188 | ClinVar, Ensembl, |
| 4 | g.187130393A > G | CYP4V2 | rs202204817 | HGMD, |
| 4 | g.187130414G > A | CYP4V2 | rs144109267 | HGMD, |
| 4 | g.187130417G > A | CYP4V2 | | HGMD, |
| 4 | g.187130420C > T | CYP4V2 | | HGMD, |
| 4 | g.39274682G > A | WDR19 | | HGMD, |
| 4 | g.6288894T > C | WFS1 | | HGMD, |
| 4 | g.6290718A > G | WFS1 | rs71530914 | HGMD, |
| 4 | g.6290726A > T | WFS1 | | HGMD, |
| 4 | g.6293062C > T | WFS1 | | HGMD, |
| 4 | g.6293068G > A | WFS1 | | HGMD, |
| 4 | g.6293094A > G | WFS1 | rs138682654 | HGMD, |
| 4 | g.6302396T > C | WFS1 | | HGMD, |
| 4 | g.656302A > G | PDE6B | | HGMD, |
| 4 | g.659043G > A | PDE6B | | HGMD, |
| 5 | g.149277970T > A | PDE6A | | HGMD, |
| 5 | g.178413133G > A | GRM6 | rs62638624 | ClinVar, Ensembl, |
| 5 | g.178413165T > A | GRM6 | | HGMD, |
| 5 | g.178413189del | GRM6 | | HGMD, |
| 5 | g.33944917C > T | SLC45A2 | | HGMD, |
| 5 | g.33944928C > T | SLC45A2 | | HGMD, |
| 5 | g.33954494A > C | SLC45A2 | | HGMD, |
| 5 | g.33964078C > G | SLC45A2 | | HGMD, |
| 5 | g.33964091C > T | SLC45A2 | | HGMD, |
| 5 | g.33982352G > A | SLC45A2 | | HGMD, |
| 5 | g.33984312A > T | SLC45A2 | | HGMD, |
| 5 | g.33984361C > T | SLC45A2 | | HGMD, |
| 5 | g.90007139G > C | GPR98 | | HGMD, |
| 5 | g.90079142G > T | GPR98 | | HGMD, |
| 5 | g.90098692G > A | GPR98 | rs61731030 | HGMD, |
| 5 | g.90124769T > G | GPR98 | | HGMD, |
| 6 | g.10830803C > G | MAK | | HGMD, |
| 6 | g.137143923C > G | PEX7 | rs61753238 | ClinVar, |
| 6 | g.1610966dup | FOXC1 | | HGMD, |
| 6 | g.1610997del | FOXC1 | | HGMD, |
| 6 | g.1611005A > G | FOXC1 | | HGMD, |
| 6 | g.1611015del | FOXC1 | | HGMD, |
| 6 | g.1611015T > C | FOXC1 | rs104893951 | Ensembl, |
| 6 | g.1611024A > C | FOXC1 | | HGMD, |
| 6 | g.1611038C > T | FOXC1 | rs121909339 | Ensembl, |
| 6 | g.1611043del | FOXC1 | | HGMD, |
| 6 | g.1611047C > T | FOXC1 | | HGMD, |
| 6 | g.1611058C > G | FOXC1 | | HGMD, |
| 6 | g.1611060G > A | FOXC1 | | HGMD, |
| 6 | g.1611063A > G | FOXC1 | | HGMD, |
| 6 | g.1611068C > T | FOXC1 | rs121909338 | Ensembl, |
| 6 | g.1611072C > T | FOXC1 | rs104893957 | Ensembl, |
| 6 | g.1611084G > A | FOXC1 | | HGMD, |
| 6 | g.31907147T > C | C2 | rs550605 | Ensembl, |
| 6 | g.31915532C > T | CFB | rs4151670 | Ensembl, |
| 6 | g.35471382T > C | TULP1 | | HGMD, |
| 6 | g.35471401A > C | TULP1 | | HGMD, |
| 6 | g.35477409A > G | TULP1 | | HGMD, |
| 6 | g.42141500C > T | GUCA1A | rs104893968 | ClinVar, Ensembl, |
| 6 | g.42141500T > C | GUCA1A | rs104893968 | HGMD, |
| 6 | g.42146986C > T | GUCA1A | rs121434631 | ClinVar, Ensembl, |
| 6 | g.42146986T > C | GUCA1A | rs121434631 | HGMD, |
| 6 | g.42146999C > A | GUCA1A | | HGMD, |
| 6 | g.42146999G > A | GUCA1A | | HGMD, |
| 6 | g.42147011T > G | GUCA1A | | HGMD, |
| 6 | g.42672106_42672107del | PRPH2 | | HGMD, |
| 6 | g.42672129A > G | PRPH2 | rs62645936 | HGMD, |
| 6 | g.42672134A > G | PRPH2 | rs62645935 | HGMD, |
| 6 | g.42933096G > A | PEX6 | | HGMD, |
| 6 | g.42933788G > A | PEX6 | | HGMD, |
| 6 | g.42935279C > T | PEX6 | | HGMD, |
| 6 | g.42935302C > A | PEX6 | | HGMD, |
| 6 | g.42936027C > T | PEX6 | | HGMD, |
| 6 | g.42936070G > A | PEX6 | | HGMD, |
| 6 | g.42937405del | PEX6 | | HGMD, |
| 6 | g.42941826T > C | PEX6 | | HGMD, |
| 6 | g.42942745del | PEX6 | | HGMD, |
| 6 | g.65149056del | EYS | | HGMD, |
| 6 | g.65303193T > A | EYS | rs190009374 | Ensembl, HGMD, |
| 6 | g.65336015del | EYS | | HGMD, |
| 6 | g.65596736C > A | EYS | | HGMD, |
| 6 | g.66005755C > G | EYS | | HGMD, |
| 6 | g.80202268C > T | LCA5 | | HGMD, |
| 7 | g.128038485A > G | IMPDH1 | rs121912551 | HGMD, |
| 7 | g.128038485C > T | IMPDH1 | rs121912551 | ClinVar, Ensembl, |
| 7 | g.128038511A > G | IMPDH1 | | HGMD, |
| 7 | g.128038575G > A | IMPDH1 | | HGMD, |
| 7 | g.128038580G > A | IMPDH1 | | GEDi, |
| 7 | g.128038595C > G | IMPDH1 | | HGMD, |
| 7 | g.128038607C > T | IMPDH1 | | HGMD, |
| 7 | g.128038611A > G | IMPDH1 | rs121912550 | HGMD, |
| 7 | g.128038611C > T | IMPDH1 | rs121912550 | ClinVar, Ensembl, |
| 7 | g.128038616C > G | IMPDH1 | rs121912552 | ClinVar, Ensembl, HGMD, |
| 7 | g.128040174A > C | IMPDH1 | rs121912554 | ClinVar, Ensembl, |
| 7 | g.128040174G > T | IMPDH1 | rs121912554 | HGMD, |
| 7 | g.128040882G > A | IMPDH1 | rs121912553 | ClinVar, Ensembl, |
| 7 | g.128040882T > C | IMPDH1 | rs121912553 | HGMD, |
| 7 | g.128414670G > A | OPN1SW | | HGMD, |
| 7 | g.33135003G > A | RP9 | rs104894039 | HGMD, |
| 7 | g.33135003T > C | RP9 | rs104894039 | ClinVar, Ensembl, |
| 7 | g.33136162T > A | RP9 | rs104894037 | ClinVar, Ensembl, HGMD, |
| 7 | g.33573603T > A | BBS9 | | HGMD, |
| 7 | g.92123607C > A | PEX1 | | HGMD, |
| 7 | g.92134045C > A | PEX1 | | HGMD, |
| 7 | g.92146589C > A | PEX1 | | HGMD, |
| 7 | g.92146636del | PEX1 | | HGMD, |
| 8 | g.100844596G > T | VPS13B | rs386834119 | ClinVar, HGMD, |
| 8 | g.100871535G > A | VPS13B | rs386834057 | ClinVar, |
| 8 | g.38874730A > G | ADAM9 | | HGMD, |
| 8 | g.38884330G > A | ADAM9 | | HGMD, |
| 8 | g.55534132A > C | RP1 | | HGMD, |
| 8 | g.55534713A > G | RPI | rs145691085 | HGMD, |
| 8 | g.55534723del | RP1 | | HGMD, |
| 8 | g.87591437del | CNGB3 | | HGMD, |
| 8 | g.87591479G > A | CNGB3 | | FIGMD, |
| 8 | g.87645092A > G | CNGB3 | rs147876778 | HGMD, |
| 8 | g.87656917A > C | CNGB3 | | HGMD, |
| 8 | g.87679359G > A | CNGB3 | | HGMD, |
| 8 | g.87683218_87683219insA | CNGB3 | | HGMD, |
| 9 | g.103055321C > T | INVS | | HGMD, |
| 9 | g.117241027del | DFNB31 | rs397517258 | Ensembl, |
| 9 | g.12698611G > A | TYRP1 | | HGMD, |
| 9 | g.12702424G > A | TYRP1 | rs281865424 | ClinVar, Ensembl, |
| 9 | g.139327731C > G | INPP5E | | HGMD, |
| 9 | g.2719087A > T | KCNV2 | | HGMD, |
| 9 | g.2719087G > T | KCNV2 | | HGMD, |
| 9 | g.2729465A > G | KCNV2 | rs104894115 | HGMD, |
| 9 | g.2729465G > A | KCNV2 | rs104894115 | ClinVar, Ensembl, |
| 9 | g.2729470A > G | KCNV2 | rs149648640 | HGMD, |
| 9 | g.2729470T > G | KCNV2 | | HGMD, |
| 9 | g.2729494del | KCNV2 | | HGMD, |
| 10 | g.102510581C > T | PAX2 | | HGMD, |
| 10 | g.102510627C > A | PAX2 | | HGMD, |
| 10 | g.102566202C > G | PAX2 | | HGMD, |
| 10 | g.126086520C > A | OAT | rs1800456 | ClinVar, Ensembl, |
| 10 | g.126086520C > G | OAT | rs1800456 | ClinVar, Ensembl, |
| 10 | g.126086524A > T | OAT | rs386833598 | ClinVar, Ensembl, |
| 10 | g.126086555G > A | OAT | rs121965058 | ClinVar, Ensembl, |
| 10 | g.126086581G > A | OAT | rs121965044 | ClinVar, Ensembl, |
| 10 | g.126086626A > G | OAT | rs121965043 | ClinVar, Ensembl, |
| 10 | g.126086630C > A | OAT | rs121965055 | ClinVar, Ensembl, |
| 10 | g.126089444C > G | OAT | rs121965045 | ClinVar, Ensembl, |
| 10 | g.126089450C > T | OAT | rs386833595 | ClinVar, Ensembl, |
| 10 | g.126089510C > T | OAT | rs121965053 | ClinVar, Ensembl, |
| 10 | g.126090315C > T | OAT | rs121965047 | ClinVar, Ensembl, |
| 10 | g.126090318G > A | OAT | rs386833623 | ClinVar, Ensembl, |
| 10 | g.126090331A > T | OAT | rs386833622 | ClinVar, Ensembl, |
| 10 | g.126090354G > A | OAT | rs121965049 | ClinVar, Ensembl, |
| 10 | g.126090357C > T | OAT | rs386833621 | ClinVar, Ensembl, |
| 10 | g.126090357del | OAT | rs386833620 | ClinVar, Ensembl, HGMD, |
| 10 | g.126091499G > C | OAT | rs121965057 | ClinVar, Ensembl, |
| 10 | g.126091572C > T | OAT | rs267606924 | ClinVar, Ensembl, |
| 10 | g.126091584C > T | OAT | rs121965042 | ClinVar, Ensembl, |
| 10 | g.126092390G > A | OAT | rs386833617 | ClinVar, Ensembl, |
| 10 | g.126092390G > T | OAT | rs386833617 | ClinVar, Ensembl, |
| 10 | g.126100738C > T | OAT | rs121965034 | ClinVar, Ensembl, |
| 10 | g.126100738del | OAT | | HGMD, |
| 10 | g.13151270G > A | OPTN | rs28939688 | Ensembl, |
| 10 | g.13158364G > A | OPTN | | HGMD, |
| 10 | g.13178766G > A | OPTN | rs75654767 | Ensembl, |
| 10 | g.13325695G > A | PHYH | rs104894178 | Ensembl, |
| 10 | g.13330512G > T | PHYH | rs28939672 | Ensembl, |
| 10 | g.13330543T > C | PHYH | | HGMD, |
| 10 | g.48385854A > G | RBP3 | rs146150511 | HGMD, |
| 10 | g.48385854C > T | RBP3 | rs146150511 | ClinVar, Ensembl, |
| 10 | g.55912907G > C | PCDH15 | | HGMD, |
| 10 | g.56077174G > A | PCDH15 | rs111033260 | Ensembl, |
| 10 | g.56077174T > C | PCDH15 | rs111033260 | HGMD, |
| 10 | g.73269965del | CDH23 | | HGMD, |
| 10 | g.73269982G > C | CDH23 | | HGMD, |
| 10 | g.73330661A > G | CDH23 | | HGMD, |
| 10 | g.73330661G > A | CDH23 | | FIGMD, |
| 10 | g.73454017G > A | CDH23 | | HGMD, |
| 10 | g.73485263T > C | CDH23 | | HGMD, |
| 10 | g.73539199T > C | CDH23 | | HGMD, |
| 10 | g.73544858G > A | CDH23 | rs397517341 | Ensembl, |
| 10 | g.73550888G > C | CDH23 | | HGMD, |
| 10 | g.73559011T > C | CDH23 | | HGMD, |
| 10 | g.73563128G > A | CDH23 | rs202052174 | Ensembl, |
| 10 | g.73563177G > A | CDH23 | | HGMD, |
| 10 | g.73567085A > G | CDH23 | | HGMD, |
| 10 | g.85961671A > G | CDHR1 | | HGMD, |
| 10 | g.85971975G > C | CDHR1 | rs143662988 | HGMD, |
| 10 | g.86007463A > C | RGR | rs104894187 | ClinVar, Ensembl, |
| 10 | g.86007463C > A | RGR | rs104894187 | HGMD, |
| 10 | g.94368821G > T | KIF11 | | HGMD, |
| 10 | g.94405401T > C | KIF11 | | HGMD, |
| 10 | g.95380377T > A | PDE6C | | HGMD, |
| 10 | g.95399825A > G | PDE6C | | HGMD, |
| 11 | g.17517141C > T | USH1C | | HGMD, |
| 11 | g.17531360G > A | USH1C | | HGMD, |
| 11 | g.17542894T > C | USH1C | | HGMD, |
| 11 | g.17547892A > C | USH1C | | HGMD, |
| 11 | g.17547896A > C | USH1C | | HGMD, |
| 11 | g.17548589del | USH1C | | HGMD, |
| 11 | g.17552719del | USH1C | | HGMD, |
| 11 | g.31812383G > C | PAX6 | rs121907915 | Ensembl, |
| 11 | g.31815345C > T | PAX6 | rs121907929 | Ensembl, |
| 11 | g.31823109G > T | PAX6 | rs121907928 | ClinVar, |
| 11 | g.31823159G > A | PAX6 | rs121907914 | ClinVar, |
| 11 | g.31824256A > G | PAX6 | | HGMD, |
| 11 | g.31824281G > A | PAX6 | rs397514640 | ClinVar, |
| 11 | g.31824286C > G | PAX6 | | HGMD, |
| 11 | g.61719283A > C | BEST1 | | HGMD, |
| 11 | g.61719283T > C | BEST1 | | HGMD, |
| 11 | g.61719286C > T | BEST1 | | HGMD, |
| 11 | g.61719289T > C | BEST1 | | HGMD, |
| 11 | g.61719291C > T | BEST1 | | HGMD, |
| 11 | g.61719295A > C | BEST1 | | HGMD, |
| 11 | g.61719295G > C | BEST1 | rs281865204 | HGMD, |
| 11 | g.61719304A > T | BEST1 | | HGMD, |
| 11 | g.61719304C > T | BEST1 | rs281865205 | HGMD, |
| 11 | g.61719307T > C | BEST1 | rs281865207 | HGMD, |
| 11 | g.61729746dup | BEST1 | | HGMD, |
| 11 | g.66281934G > T | BBS1 | | HGMD, |
| 11 | g.66283014C > T | BBS1 | | HGMD, |
| 11 | g.66291353G > A | BBS1 | | HGMD, |
| 11 | g.66299149T > A | BBS1 | | HGMD, |
| 11 | g.66299160del | BBS1 | | HGMD, |
| 11 | g.66299163G > T | BBS1 | rs121917777 | Ensembl, |
| 11 | g.6636785A > T | TPP1 | rs121908198 | ClinVar, Ensembl, |
| 11 | g.6636793G > C | TPP1 | | HGMD, |
| 11 | g.6637557A > G | TPP1 | | HGMD, |
| 11 | g.6637564T > G | TPP1 | rs121908206 | ClinVar, Ensembl, |
| 11 | g.6637573G > A | TPP1 | | HGMD, |
| 11 | g.6638858G > A | TPP1 | | HGMD, |
| 11 | g.68157389G > T | LRP5 | rs121908666 | ClinVar, |
| 11 | g.68183946A > G | LRP5 | | HGMD, |
| 11 | g.68197163T > G | LRP5 | | HGMD, |
| 11 | g.68204455A > G | LRP5 | rs28939709 | HGMD, |
| 11 | g.68204455G > A | LRP5 | rs28939709 | Ensembl, |
| 11 | g.68207386T > G | LRP5 | rs80358322 | Ensembl, HGMD, |
| 11 | g.68216290C > G | LRP5 | rs149645175 | ClinVar, |
| 11 | g.68216290C > T | LRP5 | rs149645175 | ClinVar, |
| 11 | g.76853809A > G | MYO7A | | HGMD, |
| 11 | g.76853813A > C | MYO7A | | HGMD, |
| 11 | g.76853829A > C | MYO7A | rs35689081 | HGMD, |
| 11 | g.76853829C > A | MYO7A | rs35689081 | Ensembl, |
| 11 | g.76853829C > T | MYO7A | rs35689081 | Ensembl, |
| 11 | g.76853873G > A | MYO7A | rs397516284 | Ensembl, |
| 11 | g.76866985A > C | MYO7A | | HGMD, |
| 11 | g.76867705G > A | MYO7A | | HGMD, |
| 11 | g.76873170C > G | MYO7A | | HGMD, |
| 11 | g.76873192C > T | MYO7A | rs111033286 | Ensembl, |
| 11 | g.76873192T > C | MYO7A | rs111033286 | HGMD, |
| 11 | g.76873898G > C | MYO7A | | HGMD, |
| 11 | g.76873900A > G | MYO7A | rs111033206 | HGMD, |
| 11 | g.76873900G > A | MYO7A | rs111033206 | Ensembl, |
| 11 | g.76873907del | MYO7A | | HGMD, |
| 11 | g.76877208G > A | MYO7A | rs121965082 | Ensembl, |
| 11 | g.76883791C > G | MYO7A | | HGMD, |
| 11 | g.76883795del | MYO7A | | HGMD, |
| 11 | g.76885811T > C | MYO7A | | HGMD, |
| 11 | g.76885820del | MYO7A | | HGMD, |
| 11 | g.76885835T > C | MYO7A | | HGMD, |
| 11 | g.76892417A > G | MYO7A | | HGMD, |
| 11 | g.76893448G > A | MYO7A | | HGMD, |
| 11 | g.76893467A > G | MYO7A | | HGMD, |
| 11 | g.76900388G > C | MYO7A | | HGMD, |
| 11 | g.76900393A > G | MYO7A | rs111033214 | HGMD, |
| 11 | g.76900393G > A | MYO7A | rs111033214 | Ensembl, |
| 11 | g.76901065del | MYO7A | | HGMD, |
| 11 | g.76901086A > G | MYO7A | rs111033195 | HGMD, |
| 11 | g.76901093T > C | MYO7A | | HGMD, |
| 11 | g.76901895del | MYO7A | | HGMD, |
| 11 | g.76905412del | MYO7A | | HGMD, |
| 11 | g.76912644G > C | MYO7A | | HGMD, |
| 11 | g.76919822del | MYO7A | | HGMD, |
| 11 | g.76919826A > G | MYO7A | rs111033175 | Ensembl, |
| 11 | g.76919840C > T | MYO7A | | HGMD, |
| 11 | g.76919849G > A | MYO7A | | HGMD, |
| 11 | g.89017940G > T | TYR | | HGMD, |
| 11 | g.89017949A > C | TYR | | HGMD, |
| 11 | g.89017949A > G | TYR | | HGMD, |
| 11 | g.89017949A > T | TYR | | HGMD, |
| 11 | g.89017952del | TYR | | HGMD, |
| 11 | g.89017956G > T | TYR | | HGMD, |
| 11 | g.89017961G > A | TYR | rs1126809 | ClinVar, Ensembl, |
| 11 | g.89017961G > A | TYR | rs62645918 | ClinVar, Ensembl, |
| 11 | g.89017963del | TYR | | HGMD, |
| 11 | g.89017967A > C | TYR | | HGMD, |
| 11 | g.89017970G > T | TYR | | HGMD, |
| 11 | g.89017973C > T | TYR | rs104894313 | ClinVar, Ensembl, |
| 11 | g.89017980A > C | TYR | | HGMD, |
| 11 | g.89017983A > C | TYR | | HGMD, |
| 11 | g.89017987T > C | TYR | | HGMD, |
| 11 | g.89017990C > G | TYR | | HGMD, |
| 11 | g.89017993G > T | TYR | | HGMD, |
| 11 | g.89018002G > T | TYR | | HGMD, |
| 11 | g.89018006C > A | TYR | | HGMD, |
| 11 | g.89018011G > A | TYR | rs61754392 | ClinVar, Ensembl, |
| 11 | g.89018021G > A | TYR | rs61754393 | Ensembl, |
| 11 | g.89018027C > T | TYR | | HGMD, |
| 11 | g.89018033T > A | TYR | | HGMD, |
| 11 | g.89018036T > G | TYR | | HGMD, |
| 11 | g.89018048C > T | TYR | | HGMD, |
| 11 | g.89018055C > G | TYR | | HGMD, |
| 11 | g.89018057G > T | TYR | | HGMD, |
| 11 | g.89018059A > G | TYR | | HGMD, |
| 11 | g.89018062G > C | TYR | | HGMD, |
| 11 | g.89018065G > A | TYR | | HGMD, |
| 11 | g.89018080T > C | TYR | | HGMD, |
| 11 | g.89018087A > G | TYR | | HGMD, |
| 11 | g.89018092G > A | TYR | rs104894317 | ClinVar, Ensembl, |
| 11 | g.89018098G > A | TYR | rs104894318 | ClinVar, Ensembl, |
| 11 | g.89018098G > C | TYR | rs104894318 | ClinVar, Ensembl, |
| 11 | g.89018102A > G | TYR | | HGMD, |
| 11 | g.89018108A > G | TYR | | HGMD, |
| 11 | g.89018110C > G | TYR | | HGMD, |
| 11 | g.89018113C > T | TYR | | HGMD, |
| 11 | g.89018123G > A | TYR | | HGMD, |
| 11 | g.89028369G > A | TYR | | HGMD, |
| 11 | g.89028386C > A | TYR | | HGMD, |
| 11 | g.89028413C > A | TYR | | HGMD, |
| 11 | g.89028413C > G | TYR | | HGMD, |
| 11 | g.89028460C > T | TYR | | HGMD, |
| 12 | g.15130981C > G | PDE6H | rs200311463 | ClinVar, Ensembl, |
| 12 | g.48367873G > A | COL2A1 | rs121912886 | ClinVar, |
| 12 | g.48368618C > T | COL2A1 | rs121912887 | Ensembl, |
| 12 | g.48368626del | COL2A1 | | HGMD, |
| 12 | g.48369363del | COL2A1 | | HGMD, |
| 12 | g.48369826C > G | COL2A1 | rs121912883 | ClinVar, |
| 12 | g.48369848del | COL2A1 | | HGMD, |
| 12 | g.48379729C > T | COL2A1 | rs121912888 | ClinVar, |
| 12 | g.48381393del | COL2A1 | | HGMD, |
| 12 | g.48387608C > T | COL2A1 | rs121912877 | ClinVar, |
| 12 | g.48389687G > A | COL2A1 | rs121912869 | ClinVar, Ensembl, |
| 12 | g.48391407del | COL2A1 | | HGMD, |
| 12 | g.48391428del | COL2A1 | | HGMD, |
| 12 | g.56115692T > G | RDH5 | rs104894373 | Ensembl, |
| 12 | g.56117812G > A | RDH5 | rs62638191 | Ensembl, |
| 12 | g.56117812G > T | RDH5 | rs62638191 | Ensembl, |
| 12 | g.56117818dup | RDH5 | | HGMD, |
| 12 | g.88456549del | CEP290 | | HGMD, |
| 12 | g.88456563A > C | CEP290 | | HGMD, |
| 12 | g.88474139del | CEP290 | | HGMD, |
| 12 | g.88482810del | CEP290 | | HGMD, |
| 12 | g.88482837del | CEP290 | | HGMD, |
| 12 | g.88494960T > C | CEP290 | rs281865192 | GEDi, ClinVar, HGMD, |
| 12 | g.88504978C > A | CEP290 | | HGMD, |
| 12 | g.88514052del | CEP290 | | HGMD, |
| 12 | g.88520203del | CEP290 | | HGMD, |
| 12 | g.88534732C > A | CEP290 | | HGMD, |
| 12 | g.88534777C > A | CEP290 | | HGMD, |
| 13 | g.77569368G > C | CLN5 | rs202146713 | ClinVar, |
| 14 | g.21770667del | RPGRIP1 | | HGMD, |
| 14 | g.21771701T > C | RPGRIP1 | | HGMD, |
| 14 | g.21780005C > T | RPGRIP1 | | HGMD, |
| 14 | g.21790154T > C | RPGRIP1 | rs147586703 | HGMD, |
| 14 | g.21793543G > A | RPGRIP1 | | HGMD, |
| 14 | g.21794039T > C | RPGRIP1 | rs142796310 | HGMD, |
| 14 | g.21811346del | RPGRIP1 | | HGMD, |
| 14 | g.57271048dup | OTX2 | | HGMD, |
| 14 | g.68191285T > C | RDH12 | | HGMD, |
| 14 | g.68191297G > T | RDH12 | | HGMD, |
| 14 | g.68191299C > G | RDH12 | | HGMD, |
| 14 | g.68191305C > T | RDH12 | rs104894471 | ClinVar, Ensembl, |
| 14 | g.68191305T > C | RDH12 | rs104894471 | HGMD, |
| 14 | g.68191944T > C | RDH12 | | HGMD, |
| 14 | g.68192773T > A | RDH12 | | HGMD, |
| 14 | g.68193700A > C | RDH12 | rs104894475 | HGMD, |
| 14 | g.68193700C > A | RDH12 | rs104894475 | ClinVar, Ensembl, |
| 14 | g.68193700C > G | RDH12 | rs104894475 | ClinVar, Ensembl, |
| 14 | g.68193700G > C | RDH12 | rs104894475 | HGMD, |
| 14 | g.68193703A > T | RDH12 | | HGMD, |
| 14 | g.68193713C > T | RDH12 | rs121434337 | ClinVar, Ensembl, |
| 14 | g.68193713T > C | RDH12 | rs121434337 | HGMD, |
| 14 | g.68193730T > C | RDH12 | | HGMD, |
| 14 | g.68193755A > G | RDH12 | | HGMD, |
| 14 | g.68196070C > T | RDH12 | | HGMD, |
| 14 | g.68196072T > G | RDH12 | | HGMD, |
| 14 | g.68196093T > G | RDH12 | | GEDi, |
| 14 | g.74969584C > T | LTBP2 | | HGMD, |
| 14 | g.74969614C > T | LTBP2 | rs137854860 | ClinVar, Ensembl, |
| 14 | g.74969955G > A | LTBP2 | rs387907174 | ClinVar, |
| 14 | g.74995814T > G | LTBP2 | rs137854859 | Ensembl, |
| 14 | g.75018036del | LTBP2 | | HGMD, |
| 14 | g.75019595del | LTBP2 | | HGMD, |
| 14 | g.88904181G > A | SPATA7 | | HGMD, |
| 14 | g.89307541G > A | TTC8 | | HGMD, |
| 14 | g.92343965G > A | FBLN5 | rs28939073 | ClinVar, Ensembl, |
| 14 | g.92343965T > C | FBLN5 | rs28939073 | HGMD, |
| 14 | g.92403527T > C | FBLN5 | rs141200859 | HGMD, |
| 15 | g.28096537A > G | OCA2 | | HGMD, |
| 15 | g.28096539G > T | OCA2 | | HGMD, |
| 15 | g.28096543C > G | OCA2 | | HGMD, |
| 15 | g.28096543C > T | OCA2 | | HGMD, |
| 15 | g.28116386G > A | OCA2 | | HGMD, |
| 15 | g.28116392C > A | OCA2 | | HGMD, |
| 15 | g.28202743G > A | OCA2 | rs1800413 | Ensembl, |
| 15 | g.28230211T > C | OCA2 | | HGMD, |
| 15 | g.28230225G > A | OCA2 | | HGMD, |
| 15 | g.28230225G > T | OCA2 | | HGMD, |
| 15 | g.28230238T > C | OCA2 | | HGMD, |
| 15 | g.28259941T > C | OCA2 | | HGMD, |
| 15 | g.28259965G > A | OCA2 | rs121918168 | ClinVar, Ensembl, |
| 15 | g.28273067C > A | OCA2 | | HGMD, |
| 15 | g.28326993G > A | OCA2 | | HGMD, |
| 15 | g.31324894T > G | TRPM1 | | HGMD, |
| 15 | g.31327748C > T | TRPM1 | | HGMD, |
| 15 | g.31332320C > T | TRPM1 | | HGMD, |
| 15 | g.31334214T > G | TRPM1 | | HGMD, |
| 15 | g.31353650A > C | TRPM1 | | HGMD, |
| 15 | g.31360093del | TRPM1 | | HGMD, |
| 15 | g.65944981C > T | SLC24A1 | | HGMD, |
| 15 | g.68504188G > A | CLN6 | | HGMD, |
| 15 | g.68504192G > A | CLN6 | | HGMD, |
| 16 | g.73023673C > T | BBS4 | | HGMD, |
| 15 | g.73029876C > T | BBS4 | | HGMD, |
| 15 | g.78403513C > G | CIB2 | rs145415848 | ClinVar, Ensembl, HGMD, |
| 15 | g.78403513C > T | CIB2 | rs145415848 | ClinVar, Ensembl, |
| 15 | g.89753638del | RLBP1 | | HGMD, |
| 15 | g.89754981A > T | RLBP1 | rs137853291 | Ensembl, HGMD, |
| 15 | g.89760364G > T | RLBP1 | | HGMD, |
| 15 | g.89760390T > C | RLBP1 | rs200725857 | HGMD, |
| 15 | g.89761794A > G | RLBP1 | | HGMD, |
| 16 | g.16244082T > A | ABCC6 | rs72653751 | Ensembl, |
| 16 | g.16244129T > C | ABCC6 | rs212097 | Ensembl, |
| 16 | g.16244424G > C | ABCC6 | rs72664215 | Ensembl, |
| 16 | g.16244461G > A | ABCC6 | rs63750763 | Ensembl, |
| 16 | g.16244463G > A | ABCC6 | rs72547524 | ClinVar, Ensembl, |
| 16 | g.16248476C > T | ABCC6 | rs58760581 | Ensembl, |
| 16 | g.16248495C > T | ABCC6 | rs63751241 | Ensembl, |
| 16 | g.16248501G > A | ABCC6 | rs66913554 | Ensembl, |
| 16 | g.16251627del | ABCC6 | rs72664233 | ClinVar, HGMD, |
| 16 | g.16251627dup | ABCC6 | | HGMD, |
| 16 | g.16251667C > T | ABCC6 | rs63750273 | ClinVar, Ensembl, HGMD, |
| 16 | g.16255205C > T | ABCC6 | rs2376957 | Ensembl, |
| 16 | g.16255240G > A | ABCC6 | rs3213470 | Ensembl, |
| 16 | g.16256992del | ABCC6 | | HGMD, |
| 16 | g.16256994G > A | ABCC6 | rs63750987 | Ensembl, |
| 16 | g.16256994G > C | ABCC6 | rs63750987 | Ensembl, |
| 16 | g.16257016G > A | ABCC6 | rs63749794 | Ensembl, |
| 16 | g.16263524C > G | ABCC6 | rs72657692 | Ensembl, |
| 16 | g.16267111T > C | ABCC6 | rs72664301 | Ensembl, |
| 16 | g.16267140C > A | ABCC6 | rs72664209 | ClinVar, Ensembl, HGMD, |
| 16 | g.16272773G > T | ABCC6 | rs72653789 | Ensembl, |
| 16 | g.16272777G > A | ABCC6 | | HGMD, |
| 16 | g.16272792G > A | ABCC6 | rs72653788 | Ensembl, |
| 16 | g.16272807C > T | ABCC6 | rs72653787 | Ensembl, |
| 16 | g.16272818A > T | ABCC6 | rs72653786 | Ensembl, |
| 16 | g.16276419C > A | ABCC6 | rs72653784 | Ensembl, |
| 16 | g.16276423T > G | ABCC6 | rs72653783 | Ensembl, |
| 16 | g.16276656C > T | ABCC6 | rs72664208 | Ensembl, HGMD, |
| 16 | g.16278896A > C | ABCC6 | rs72664207 | Ensembl, HGMD, |
| 16 | g.16280990dup | ABCC6 | | HGMD, |
| 16 | g.16284053A > G | ABCC6 | rs72653773 | Ensembl, |
| 16 | g.16284093C > G | ABCC6 | | HGMD, |
| 16 | g.16292022G > C | ABCC6 | | HGMD, |
| 16 | g.16292024T > C | ABCC6 | rs72653764 | Ensembl, |
| 16 | g.16295852G > A | ABCC6 | rs72664295 | Ensembl, |
| 16 | g.16295858C > A | ABCC6 | rs72653763 | Ensembl, |
| 16 | g.16295858C > G | ABCC6 | rs72653763 | Ensembl, |
| 16 | g.16295863T > C | ABCC6 | rs72653762 | Ensembl, |
| 16 | g.16295890G > A | ABCC6 | rs72653761 | Ensembl, |
| 16 | g.16295893A > G | ABCC6 | rs72664284 | Ensembl, |
| 16 | g.16295902G > A | ABCC6 | rs72650699 | Ensembl, |
| 16 | g.16295926T > C | ABCC6 | rs72653760 | Ensembl, |
| 16 | g.16295943G > C | ABCC6 | rs72653759 | Ensembl, |
| 16 | g.16295947G > A | ABCC6 | rs72650698 | Ensembl, |
| 16 | g.16295957T > C | ABCC6 | rs72664283 | Ensembl, |
| 16 | g.16295970A > C | ABCC6 | rs72653758 | Ensembl, |
| 16 | g.16297265del | ABCC6 | | HGMD, |
| 16 | g.16297305del | ABCC6 | | HGMD, |
| 16 | g.16297310T > C | ABCC6 | rs72657699 | Ensembl, |
| 16 | g.16297314G > C | ABCC6 | rs78678589 | Ensembl, |
| 16 | g.16297314G > T | ABCC6 | rs78678589 | Ensembl, |
| 16 | g.16297410G > A | ABCC6 | rs4780605 | Ensembl, |
| 16 | g.16297424T > C | ABCC6 | rs4780606 | Ensembl, |
| 16 | g.16302586T > C | ABCC6 | rs72657698 | Ensembl, |
| 16 | g.16302625G > A | ABCC6 | rs72653757 | Ensembl, |
| 16 | g.16302637G > A | ABCC6 | rs72653756 | Ensembl, |
| 16 | g.16302655C > A | ABCC6 | rs72650697 | Ensembl, |
| 16 | g.16302698G > C | ABCC6 | | HGMD, |
| 16 | g.16302703C > T | ABCC6 | rs72653755 | Ensembl, |
| 16 | g.16306030G > A | ABCC6 | rs72664291 | Ensembl, |
| 16 | g.16306030G > C | ABCC6 | rs72664291 | Ensembl, |
| 16 | g.16306030G > T | ABCC6 | rs72664291 | Ensembl, |
| 16 | g.16306050C > A | ABCC6 | rs72653754 | Ensembl, |
| 16 | g.16306059C > T | ABCC6 | rs72664282 | Ensembl, |
| 16 | g.16306085C > T | ABCC6 | rs72657697 | Ensembl, |
| 16 | g.16308158G > A | ABCC6 | rs72664290 | Ensembl, |
| 16 | g.16308186G > A | ABCC6 | | HGMD, |
| 16 | g.16308232C > T | ABCC6 | rs72664281 | Ensembl, |
| 16 | g.16308285G > A | ABCC6 | | HGMD, |
| 16 | g.16308351G > A | ABCC6 | rs2239319 | Ensembl, |
| 16 | g.16313412G > A | ABCC6 | rs2606921 | Ensembl, |
| 16 | g.16313424T > C | ABCC6 | | HGMD, |
| 16 | g.16313499C > T | ABCC6 | rs72653753 | Ensembl, |
| 16 | g.16313512C > A | ABCC6 | | HGMD, |
| 16 | g.16313512C > T | ABCC6 | | HGMD, |
| 16 | g.16313545C > A | ABCC6 | rs55778939 | Ensembl, |
| 16 | g.16313545C > G | ABCC6 | rs55778939 | Ensembl, |
| 16 | g.16313545C > T | ABCC6 | rs55778939 | Ensembl, HGMD, |
| 16 | g.16313653G > A | ABCC6 | rs56019914 | Ensembl, |
| 16 | g.16313653G > C | ABCC6 | rs56019914 | Ensembl, |
| 16 | g.16313653G > T | ABCC6 | rs56019914 | Ensembl, |
| 16 | g.16313805C > G | ABCC6 | rs72664203 | Ensembl, HGMD, |
| 16 | g.16315612C > G | ABCC6 | rs72653752 | Ensembl, |
| 16 | g.16315620del | ABCC6 | | HGMD, |
| 16 | g.16315689C > T | ABCC6 | rs72657702 | Ensembl, HGMD, |
| 16 | g.28493423T > G | CLN3 | rs386833698 | ClinVar, HGMD, |
| 16 | g.28493426C > G | CLN3 | rs386833699 | ClinVar, |
| 16 | g.28493428G > A | CLN3 | rs386833697 | ClinVar, |
| 16 | g.28493434del | CLN3 | rs386833696 | ClinVar, |
| 16 | g.28495324T > G | CLN3 | rs386833738 | ClinVar, |
| 16 | g.28499055A > G | CLN3 | rs386833714 | ClinVar, |
| 16 | g.28502798C > T | CLN3 | rs386833704 | ClinVar, HGMD, |
| 16 | g.28502823C > T | CLN3 | rs386833700 | ClinVar, |
| 16 | g.53639522G > A | RPGRIP1L | rs151332923 | Ensembl, |
| 16 | g.53692694A > G | RPGRIP1L | | HGMD, |
| 16 | 9.56519633C > T | BBS2 | | HGMD, |
| 16 | g.56534792del | BBS2 | | HGMD, |
| 16 | g.56536359T > C | BBS2 | | HGMD, |
| 16 | g.56536362C > T | BBS2 | | HGMD, |
| 16 | g.56536366G > A | BBS2 | rs121908178 | Ensembl, |
| 16 | g.68712779T > C | CDH3 | | HGMD, |
| 16 | g.68713840del | CDH3 | | HGMD, |
| 16 | g.75576500C > T | TMEM231 | rs397514753 | ClinVar, |
| 17 | g.36493523del | GPR179 | | HGMD, |
| 17 | g.56290344T > C | MKS1 | | HGMD, |
| 17 | g.56292193G > A | MKS1 | rs386834049 | ClinVar, |
| 17 | g.56296510A > G | MKS1 | rs386834052 | ClinVar, HGMD, |
| 17 | g.6329126A > G | AIPL1 | | HGMD, |
| 17 | g.6331637C > T | AIPL1 | | HGMD, |
| 17 | g.6337251A > G | AIPL1 | rs62653018 | HGMD, |
| 17 | g.6337271T > C | AIPL1 | rs144822294 | HGMD, |
| 17 | g.6337279C > T | AIPL1 | rs62653017 | HGMD, |
| 17 | g.6337299A > G | AIPL1 | | HGMD, |
| 17 | g.72915558T > A | USH1G | rs397517925 | Ensembl, HGMD, |
| 17 | g.7906368A > G | GUCY2D | | HGMD, |
| 17 | g.7906368C > G | GUCY2D | | HGMD, |
| 17 | g.7909966T > C | GUCY2D | | HGMD, |
| 17 | g.7909997A > C | GUCY2D | rs61749679 | HGMD, |
| 17 | g.7912905G > T | GUCY2D | | HGMD, |
| 17 | g.7915912T > C | GUCY2D | rs34598902 | HGMD, |
| 17 | g.7916429C > T | GUCY2D | | HGMD, |
| 17 | g.7916439T > C | GUCY2D | | HGMD, |
| 17 | g.7918775del | GUCY2D | | HGMD, |
| 17 | g.7918803T > G | GUCY2D | rs61750184 | HGMD, |
| 17 | g.7918815T > A | GUCY2D | | HGMD, |
| 17 | g.7918821del | GUCY2D | | HGMD, |
| 19 | g.48339521A > G | CRX | rs61748436 | HGMD, |
| 19 | g.48339521G > A | CRX | rs61748436 | ClinVar, Ensembl, |
| 19 | g.48339523A > G | CRX | | HGMD, |
| 19 | g.54627127G > A | PRPF31 | | HGMD, |
| 19 | g.54627141T > G | PRPF31 | | HGMD, |
| 19 | g.54627153T > G | PRPF31 | | HGMD, |
| 19 | g.54633399C > G | PRPF31 | | HGMD, |
| 19 | g.6709848C > T | C3 | rs2230204 | Ensembl, |
| 20 | g.10389422T > C | MKKS | rs137853909 | Ensembl, |
| 20 | g.10623135C > A | JAG1 | | HGMD, |
| 20 | g.10623135C > T | JAG1 | | HGMD, |
| 20 | g.10623142G > A | JAG1 | | HGMD, |
| 20 | g.10626619dup | JAG1 | | HGMD, |
| 20 | g.10626634dup | JAG1 | | HGMD, |
| 20 | g.10626642C > T | JAG1 | | HGMD, |
| 20 | g.10628608C > G | JAG1 | | HGMD, |
| 20 | g.10628615del | JAG1 | | HGMD, |
| 20 | g.10629199G > A | JAG1 | | FIGMD, |
| 20 | g.10629238G > A | JAG1 | | HGMD, |
| 20 | g.10629767A > C | JAG1 | | HGMD, |
| 20 | g.10630901G > A | JAG1 | | HGMD, |
| 20 | g.10630917del | JAG1 | | HGMD, |
| 20 | g.10630924del | JAG1 | | HGMD, |
| 20 | g.10632340C > A | JAG1 | | HGMD, |
| 20 | g.10632342del | JAG1 | | HGMD, |
| 20 | g.10633116C > T | JAG1 | | HGMD, |
| 20 | g.10633136del | JAG1 | | HGMD, |
| 20 | g.10633138C > G | JAG1 | | HGMD, |
| 20 | g.10639125A > C | JAG1 | | HGMD, |
| 20 | g.10639138C > A | JAG1 | | HGMD, |
| 20 | g.10639151C > A | JAG1 | | HGMD, |
| 20 | g.10654106G > A | JAG1 | | HGMD, |
| 20 | g.10654109del | JAG1 | | HGMD, |
| 20 | g.3893281G > A | PANK2 | rs137852963 | ClinVar, |
| 20 | g.3897602C > T | PANK2 | rs137852968 | ClinVar, Ensembl, |
| 20 | g.62658491C > T | PRPF6 | rs387907100 | ClinVar, Ensembl, |
| 20 | g.62658491T > C | PRPF6 | | HGMD, |
| X | g.13753465G > A | OFD1 | rs312262808 | ClinVar. |
| X | g.13753465G > C | OFD1 | rs312262808 | ClinVar, |
| X | g.13753467T > C | OFD1 | rs312262809 | ClinVar, |
| X | g.13754606C > T | OFD1 | rs312262810 | ClinVar, |
| X | g.13756989C > T | OFD1 | rs312262825 | ClinVar, |
| X | g.13762552T > A | OFD1 | rs312262835 | ClinVar, |
| X | g.13764918del | OFD1 | rs312262844 | ClinVar, |
| X | g.13767574del | OFD1 | rs312262856 | ClinVar, |
| X | g.13767588A > T | OFD1 | rs312262857 | ClinVar, |
| X | g.13767636del | OFD1 | rs312262860 | ClinVar, |
| X | g.13768358A > G | OFD1 | | HGMD, |
| X | g.13771485A > G | OFD1 | rs312262861 | ClinVar, |
| X | g.13771485A > T | OFD1 | rs312262861 | ClinVar, |
| X | g.13773324del | OFD1 | rs312262867 | ClinVar, |
| X | g.13774778A > C | OFD1 | rs122460150 | ClinVar, |
| X | g.13774794del | OFD1 | rs312262873 | ClinVar, |
| X | g.13774833T > A | OFD1 | rs312262877 | ClinVar, |
| X | g.13775787C > T | OFD1 | rs312262880 | ClinVar, |
| X | g.13778634del | OFD1 | rs312262889 | ClinVar, |
| X | g.13779203G > T | OFD1 | rs312262892 | ClinVar, |
| X | g.13786182del | OFD1 | rs312262894 | ClinVar, HGMD, |
| X | g.13786212dup | OFD1 | | HGMD, |
| X | g.153416373A > G | OPN1LW | | HGMD, |
| X | g.153418532C > T | OPN1LW | rs150391359 | HGMD, |
| X | g.153418535G > A | OPN1LW | rs145009674 | HGMD, |
| X | g.153418541T > G | OPN1LW | rs949431 | Ensembl, |
| X | g.153420077C > T | OPN1LW | rs121434621 | HGMD, |
| X | g.153420077T > C | OPN1LW | rs121434621 | Ensembl, |
| X | g.153420209C > T | OPN1LW | rs104894912 | GEDi, Ensembl, |
| X | g.153420209T > C | OPN1LW | rs104894912 | HGMD, |
| X | g.153421944T > C | OPN1LW | | HGMD, |
| X | g.153455662C > T | OPN1MW | rs267606927 | HGMD, |
| X | g.153455662T > C | OPN1MW | rs267606927 | Ensembl, |
| X | g.153457207T > C | OPN1MW | rs104894914 | Ensembl, |
| X | g.18662723G > A | RS1 | rs199469696 | ClinVar, |
| X | g.18662737A > G | RS1 | | HGMD, |
| X | g.18665312C > A | RS1 | rs104894934 | ClinVar, Ensembl, |
| X | g.18665312C > G | RS1 | rs104894934 | ClinVar, Ensembl, HGMD, |
| X | g.18665312T > G | RS1 | | HGMD, |
| X | g.18665314G > T | RS1 | rs61752072 | HGMD, |
| X | g.18665325G > C | RS1 | rs61752070 | HGMD, |
| X | g.18665333G > A | RS1 | rs61752067 | ClinVar, Ensembl, |
| X | g.18665333T > C | RS1 | rs61752067 | HGMD, |
| X | g.18665336C > G | RS1 | rs61752066 | HGMD, |
| X | g.18665336del | RS1 | | HGMD, |
| X | g.18665344A > C | RS1 | rs61752065 | HGMD, |
| X | g.18665349A > G | RS1 | rs61752064 | HGMD, |
| X | g.18665349C > G | RS1 | | HGMD, |
| X | g.18665351A > G | RS1 | rs61752063 | ClinVar, Ensembl, |
| X | g.18665351C > T | RS1 | rs61752063 | HGMD, |
| X | g.18674860del | RS1 | | HGMD, |
| X | g.18674880T > C | RS1 | | HGMD, |
| X | g.38156535A > T | RPGR | | HGMD, |
| X | g.38156552T > C | RPGR | | HGMD, |
| X | g.38158220G > A | RPGR | | GEDi, |
| X | g.38158220T > C | RPGR | | HGMD, |
| X | g.38163888A > G | RPGR | | HGMD, |
| X | g.38163888T > G | RPGR | | HGMD, |
| X | g.38163918C > T | RPGR | rs62640589 | HGMD, |
| X | g.38169867C > G | RPGR | | HGMD, |
| X | g.38169882T > C | RPGR | | HGMD, |
| X | g.38169899del | RPGR | | HGMD, |
| X | g.38169916T > A | RPGR | rs62638653 | HGMD, |
| X | g.38182652C > T | RPGR | | HGMD, |
| X | g.38182652T > G | RPGR | rs281865296 | HGMD, |
| X | g.38182679A > G | RPGR | rs62638629 | HGMD, |
| X | g.38182684G > C | RPGR | | HGMD, |
| X | g.41332771G > A | NYX | | HGMD, |
| X | g.43809221C > A | NDP | | NDP, |
| X | g.43809224A > G | NDP | | NDP, |
| X | g.43809227G > A | NDP | | NDP, |
| X | g.43809229G > T | NDP | rs104894882 | ClinVar, NDP, |
| X | g.43809242del | NDP | | HGMD, NDP, |
| X | g.43809248C > T | NDP | | NDP, |
| X | g.43809253C > T | NDP | | NDP, |
| X | g.43809260C > A | NDP | | NDP, |
| X | g.43809262A > G | NDP | | NDP, |
| X | g.43809266A > C | NDP | | HGMD, |
| X | g.43809266G > A | NDP | rs104894880 | ClinVar, NDP, |
| X | g.43809266G > T | NDP | | NDP, |
| X | g.43809268A > T | NDP | rs104894869 | ClinVar, NDP, |
| X | g.49062114del | CACNA1F | | HGMD, |
| X | g.49062998G > A | CACNA1F | | HGMD, |
| X | g.49067069G > C | CACNA1F | | HGMD, |
| X | g.49074913C > T | CACNA1F | | HGMD, |
| X | g.49074943G > A | CACNA1F | | HGMD, |
| X | g.49076250C > G | CACNA1F | | HGMD, |
| X | g.49084895C > A | CACNA1F | | HGMD, |
| X | g.49087073T > C | CACNA1F | | HGMD, |
| X | g.85134059T > C | CHM | rs397514603 | ClinVar, Ensembl, |
| X | g.9709377C > T | GPR143 | | HGMD, |
| X | g.9709387C > A | GPR143 | | HGMD, |
| X | g.9709387C > T | GPR143 | | HGMD, |
| X | g.9709389A > C | GPR143 | | HGMD, |
| X | g.9714186G > A | GPR143 | | HGMD, |
| X | g.9714189C > T | GPR143 | | HGMD, |
| X | g.9727456T > C | GPR143 | | HGMD, |
| X | g.9728752C > G | GPR143 | | HGMD, |
| X | g.9728757C > T | GPR143 | | HGMD, |
| X | g.9728764C > T | GPR143 | | HGMD, |
| X | g.9728771A > C | GPR143 | | HGMD, |
| X | g.9728771A > G | GPR143 | | HGMD, |
| X | g.9728781dup | GPR143 | | HGMD, |
| X | g.9728784C > T | GPR143 | | HGMD, |

### Example 6. Reproducibility and Repeatability

The reproducibility of variant detection by GEDi was assessed by comparing the detection rates for the 2,443 common SNPs in all 9 GEDi datasets from each of the 4 samples. Bases that were discrepant in 1 or more of the 9 datasets for each sample in this “GEDi vs. GEDi” comparison were identified. GEDi capture followed by Illumina sequencing is highly reproducible, with only 4-6 discrepancies detected between the replicates for each DNA sample. In each case, the discrepancies related to the heterozygous vs. homozygous state of the same identified base. In the majority of cases, one out of the 9 sequence runs performed for each DNA sample contained the discrepancy (Table 5). Further analysis of the data for each of the 17 total discrepancies showed that they were due to low SNP quality score in the SAMtools variant identification software. Sixteen of these are the same as those detected in the GEDi vs. Omni 2.5 comparisons described above (Table 5).

The repeatability and reproducibility of the GEDi test was also evaluated using the kappa statistic, or kappa coefficient of agreement³². For GEDi replicates performed on the same day the kappa statistic was 0.83088, indicating almost perfect agreement between the data obtained in the three replicates for each DNA sample analyzed³². For GEDi tests performed for the 4 individual samples on each of the three separate days the kappa statistic was 0.76366, indicating excellent reproducibility³².

TABLE 5
Details of discrepancies for GEDi vs Omni 2.5 and GEDi vs. GEDi
comparisons. Discrepancies detected in the GEDi vs Omni 2.5, and GEDi vs GEDi
comparisons are indicated. The first worksheet is a summary of all discrepancy data.

| | | | | OGI-132- | | | | | |
|---|---|---|---|---|---|---|---|---|---|
| | | NA12878 | | 357 VS | OGI-281- | OGI-281-608 | OGI-307- | OGI-307-717 | |
| | NA12878 | VS OMNI | OGI-132-357 | OMNI | 608 | VS OMNI | 717 | VS OMNI | DISCREPANCY |
| chr1: | | | 2/6 HOM/HET | NO CALL | | | | | *1 |
| 94473845 | | | | | | | | | |
| chr1: | | | | | 1/9 | 1/9 | | | *1 |
| 156146218 | | | | | HOM/HET | HOM/HET | | | |
| chr1: | | | | | | | | | *1 |
| 186101539 | | | | | | | | | |
| chr1: | | | | | | | 1/9 | 1/9 HOM/HET | *1 |
| 213071341 | | | | | | | HOM/HET | | |
| chr2: | | | | | 1/9 | 1/9 | | | *1 |
| 62052380 | | | | | HOM/HET | HOM/HET | | | |
| chr2: | | | 3/8 HOM/HET | 3/8 | | | | | *1 |
| 166770120 | | | | HOM/HET | | | | | |
| chr3: | | 9/9 | | | | 9/9 | | | *2 GEDi/*2 GEDi |
| 63986047 | | HOM/HET | | | | HOM/HET | | | |
| chr3: | 2/9 | 2/9 | | | | | | | *1 |
| 150645351 | HOM/HET | HOM/HET | | | | | | | |
| chr3: | 1/5 | 1/5 | | | | | | | *1 |
| 193413502 | HOM/HET | HOM/HET | | | | | | | |
| chr4: | | | | | | | | 9/9 HOM/HET | *5 |
| 6304087 | | | | | | | | | |
| chr4: | | | | 9/9 | | | | | *4 |
| 15981874 | | | | HOM/HET | | | | | |
| chr4: | | 9/9 | | | | 9/9 | 2/7 | 2/7 HOM/HET | *1 Omni/*4 GEDi/ |
| 15982166 | | HOM/HET | | | | HOM/HET | HOM/HET | | *1 |
| chr5: | 2/9 | 2/9 | | | | | 1/9 | 1/9 HOM/HET | *1/*1 |
| 178405941 | HOM/HET | HOM/HET | | | | | HOM/HET | | |
| chr6: | 1/7 | 1/7 | 1/3 HOM/HET | 1/3 | | | 1/7 | 1/7 HOM/HET | *1/*1/*1 |
| 42932200 | HOM/HET | HOM/HET | | HOM/HET | | | HOM/HET | | |
| chr6: | 1/8 | 1/8 | 1/7 HOM/HET | 1/7 | | | | | *1/*1/*1 GEDi |
| 42932202 | HOM/HET | HOM/HET | | HO WHET | | | | | |
| chr8: | | | | | | | | | *1 GEDi |
| 10463944 | | | | | | | | | |
| chr9: | | | | | 1/9 | 1/9 | | | *1 |
| 102861613 | | | | | HOM/HET | HOM/HET | | | |
| chr9: | | | | 9/9 | | 9/9 | | 9/9 HOM/HET | *1 OMNI/*1 OMNI/ |
| 139327064 | | | | HOM/HET | | HOM/HET | | | *1 OMNI |
| chr10: | | | | | 1/9 | 1/9 | | | *1 |
| 73461805 | | | | | HOM/HET | HOM/HET | | | |
| chr10: | | 9/9 | | 9/9 | | | | | *1 OMNI/*1 OMNI |
| 85976966 | | HOM/HET | | HOM/HET | | | | | |
| chr11: | | | | 9/9 | | 9/9 | | | *2 GEDi/*2 GEM |
| 76895772 | | | | HOM/HET | | HOM/HET | | | |
| chr11: | | | | | | | | | *1 GEDi |
| 76919478 | | | | | | | | | |
| chr14: | | | | | | | | | *1 GEDi |
| 74976852 | | | | | | | | | |
| chr15: | | | | | | | | | *6 |
| 73023725 | | | | | | | | | |
| chr15: | | 9/9 | | 9/9 | | 9/9 DISCREP | | 9/9 DISCREP | *3 GEDi/*3 GEDi/ |
| 78397352 | | DISCREP | | DISCREP | | | | | *3 GEDi/*3 GEDi |
| chr16: | | | 1/7 HOM/HET | 1/7 | | | | | *1 |
| 1265600 | | | | HOM/HET | | | | | |
| chr16: | | | 1/9 HOM/HET | 1/9 | | | | | *1 |
| 1574863 | | | | HOM/HET | | | | | |
| chr16: | | | | | 1/9 | 1/9 | | | *1 |
| 57937788 | | | | | HOM/HET | HOM/HET | | | |
| chr17: | | | | | 1/9 | 1/9 | | | *1 |
| 11835331 | | | | | HOM/HET | HOM/HET | | | |

Legend for Table 5:
*1 Low SNP quality score in SAMtools
*2 HET Deletion on target base
*3 HOM Deletion close to target base
*4 HET Insertion close to target base
*5 Omni incorect; reason uncertain
*6 Misalignment in WES Sanger match with OMNI and GEDi

### Example 7. Mutation Detection

The GEDi test correctly identified mutations in 17/18 patient samples with known IRDs, glaucoma and optic atrophy variants, including 10 indels (Table 6). GEDi testing did not correctly identify the pathogenic FOXC1 indel mutation in a patient with glaucoma; however, analysis showed a design gap in FOXC1 where this pathogenic mutation is located.

TABLE 6
Data for 18 Concordance Samples

| | | Chromosome | | Mutation I | Concor- | Chromosome | | | Concor- |
|---|---|---|---|---|---|---|---|---|---|
| SubjectID | Gene | Location | Mutation I | Effect | dance | Location | Mutation II | Mutation II Effect | dance |
| OGI-027- | PDE6B | chr4: | c.2406_ | p.(Asn802del) | Yes | chr4: | c.810C > A | p.(Cys270*) | Yes |
| 065 | | 661695 | 2408delCAA | | | 647739 | | | |
| OGI-054- | EYS | chr6: | c.3243 + | p.? | Yes | chr6: | c.3243 + | p.? | Yes |
| 134 | | 65531537 | 1G > A | | | 65531537 | 1G > A | | |
| OGI-135- | C2orf71 | chr2: | c.478_479insA | p.(Cys160*) | Yes | chr2: | c.478_ | p.(Cys160*) | Yes |
| 352 | | 29296649 | | | | 29296649 | 479insA | | |
| OGI-136- | CNGB1 | chr16: | c.2544_ | p.(Leu849Alafs*) | Yes | chr16: | c.522_ | p. (Lys175Glnfs*) | Yes |
| 364 | | 57938727 | 2545insG | | | 57994755 | 523insC | | |
| OGI-140- | CYP4V2 | chr4: | c.367A > G | p.(Met123Val) | Yes | chr4: | c.367A > | p.(Met123Val) | Yes |
| 381 | | 187117196 | | | | 187117196 | G | | |
| OGI-260- | USH24 | chr1: | c.11216_11223 | p.(Leu3739Glnfs*) | Yes | chr1: | c.10561A > | p.(Trp3521Arg) | Yes |
| 546 | | 215933009 | delTGGAGCCC | | | 215956104 | G | | |
| OGI-305- | ABCA4 | chr1: | c.5882G > A | p.(Gly1961Glu) | Yes | ch1: | c.5461- | p.? | Yes |
| 713 | | 94473807 | | | | 94476951 | 10T > C | | |
| D1830 | PITX2 | chr4: | c.202C > T | p.(Gln68*) | Yes | -NA- | -NA- | -NA- | -NA- |
| | | 111542508 | | | | | | | |
| D4598 | LTBP2 | chr14: | c.956C > A | p.(Pro319Gln) | Yes | -NA- | -NA- | -NA- | -NA- |
| | | 75022271 | | | | | | | |
| D5258 | MYOC | chr1: | c.1041T > C | p.(Tyr347Tyr) | Yes | -NA- | -NA- | -NA- | -NA- |
| (aka 5728) | | 171605539 | | | | | | | |
| D23_1 | PAX6 | chr11: | c.718C > T | p.(Arg240*) | Yes | -NA- | -NA- | -NA- | -NA- |
| | | 31815627 | | | | | | | |
| D61_1 | OPTN | chr10: | c.293T > A | p.(Met98Lys) | Yes | -NA- | -NA- | -NA- | -NA- |
| | | 13152400 | | | | | | | |
| D74_1 | OPA1 | chr3: | NM_015560.2: | p.(Val903Glyfs*) | Yes | -NA- | -NA- | -NA- | -NA- |
| | | 193384959- | c.2708- | | | -NA- | | | |
| | | 193384962 | 2711delAGTT^{¥} | | | | | | |
| D98_1 | OPA1 | chr3: | c.473G > A | p.(Ser158Ans) | Yes | chr3: | c.2274T > | p.(Ala758Ala) | Yes |
| | | 193334991 | | | | 193374964 | C | | |
| D103_1 | OPA1 | chr3: | NM_015560.2: | splicing? | Yes | chr3: | NM_ | splicing? | Yes |
| | | 193355074 | c.870 + 4T > C | | | 193355074 | 015560.2; | | |
| | | | | | | | c.870 + | | |
| | | | | | | | 4T > C | | |
| OGI-273- | MT-ND4 | m: 11778 | m.11778G > A | p. (Arg340His) | Yes | -NA- | -NA- | -NA- | -NA- |
| 581 | | | | | | | | | |
| D2145 | CYP1B1 | chr2: | c.806_814del | p.(Ser269Thrfs*6) | Yes | chr2: | c.1200_ | p.(Thr404Asnfs*27) | Yes |
| | | 38301717- | GCAACTTCA | | | 38298287- | 1209dup | | |
| | | 38301726 | | | | 38298288 | TCATGCCACC | | |
| D5017 | FOXC1 | chr6: | insertion | p.? | No^{%} | -NA- | -NA- | -NA- | -NA- |
| | | 1611803- | of inframe | | | | | | |
| | | 1611852 | (CCG)n | | | | | | |

^{%}Mutation falls within a known GEDi targeted enrichment design gap
^{¥}This deletion has been reported by Delettre C. et al “Nuclear gene OPAI, encoding a mitochondrial dynamin-related protein, is mutated in dominant optic atrophy” Nat Genet. 2000 Oct;26(2): 207-10 as c.2708_2711delTTAG. NGS result: chr3: 193384956, NM_015560.2: c.2708-2delAGTT based on updated annotation rules.

### Example 8. Clinical Sensitivity

GEDi clinical sensitivity was analyzed using samples from 192 probands with diagnoses of isolated or syndromic IRD, albinism or microphthalmos (Table 7). Analyses of the sequence data identified genetic diagnoses for 98 of the probands, representing a clinical sensitivity of 51%, consistent with findings from other studies (Table 7)^{22-26.} The majority of these diagnoses were consistent with the subject's clinical presentation and family history. Two subjects without a family history of disease were found to have mutations in the known dominant IRD disease genes PRPH2 and IMPDH1, consistent with identification of de novo mutations in the affected individuals; segregation analyses confirmed de novo mutations in these two subjects (OGI-301-703 and OGI-274-582, Table 7). While de novo mutations have been reported as the cause of dominant RP, de novo mutations in the PRPH2 and IMPDH1 genes have not been previously reported. The majority of subjects that were not diagnosed genetically by GEDi testing had non-syndromic RP ( 52/89=58%; Table 8).

TABLE 7
Detailed information for subjects with genetic diagnoses identified
via GEDi testing.

| | | | | | | |
|---|---|---|---|---|---|---|
| OGI-035-090* | OGI-561-1151 | OGI-509-1049 | OGI-494-1018 | OGI-396-856 | OGI-409-877 | Patient ID |
| CD | CD | CD | CD | Best | Best | Clinical |
| | | | | | | Diagnosis¹ |
| X | R | R | R | D | D | Inheritance |
| chrX: | chr1: | chr8: | chr8: | chr11: | chr11: | Chromo- |
| 38145072 | 94574147 | 87656008 | 87641230 | 61719367 | 61724875 | somal |
| | | | | | | Position |
| RPGR | ABCA4 | CNGB3 | CNGB3 | BEST1 | BEST1 | Gene |
| c.3178_ | c.428C > T | c.1148delC | c.1397T > A | c.89A > G | c.653G > A | Mutation 1 |
| 3179delGA | | | | | | |
| p.(Glu1060Argfs*18) | p.(Pro143Leu) | p (Thr383Ilefs*13) | p.(Met466Lys) | p.(Lys30Arg) | p.(Arg218His) | Mutation 1 |
| | | | | | | Effect |
| CD064606 | CM032797 | CD001927 | This study | CM004422 | CM003486 | HGMD |
| 16936086 | 14517951 | 10888875 | This study | 10798642 | 10798642, | PubMed ID |
| | | | | | 17287362 | |
| NA/NA/P | D/P/P | NA/NA/P | D/P/P | D/P/P | D/P/P | SIFT/PolyPhen/ |
| | | | | | | Mutation |
| | | | | | | Taster |
| A1 = 29/ | Absent | A1 = 29/ | Absent | Absent | Absent | EVS |
| R = 10173 | | R = 12487 | | | | Frequency |
| -NA- | chr1: | chr8: | chr8: | -NA- | -NA- | Chromosome |
| | 94574147 | 87656008 | 87656008 | | | Position |
| -NA- | c.428C > T | c.1148delC | c.1148delC | -NA- | -NA- | Mutation 2 |
| -NA- | p.(Pro143Leu) | p.(Thr383Ilefs*13) | p.(Thr383Ilefs*13) | -NA- | -NA- | Mutation 2 |
| | | | | | | Effect |
| -NA- | CM032797 | CD001927 | CD001927 | -NA- | -NA- | HGMD |
| -NA- | 14517951 | 10888875 | 10888875 | -NA- | -NA- | PubMed ID |
| -NA- | D/P/P | NA/NA/P | NA/NA/P | -NA- | -NA- | PolyPhen/ |
| | | | | | | SIFT/Mutation |
| | | | | | | Taster² |
| -NA- | Absent | A1 = 29/ | A1 = 29/ | -NA- | -NA- | EVS |
| | | R = 12487 | R = 12487 | | | Frequency |
| NA | Yes | NA | Yes | NA | Yes | Segregation |
| | | | | | | Validated |
| OGI-431-918 | OGI-430-915 | OGI-430-914 | OGI-297-650 | OGI-563-1155 | OGI-019-047* | Patient ID |
| Ciliopathy | Ciliopathy | Ciliopathy | Ciliopathy | Chorioderemia | ChorD | Clinical |
| | | | | | | Diagnosis¹ |
| R | R | R | R | X | R | Inheritance |
| chr11: | chr12: | chr12: | chr16: | chrX: | chr14: | Chromosomal |
| 66293652 | 76740270 | 76740270 | 56530894 | 85128190 | 24551834 | Position |
| BBS1 | BBS10 | BBS10 | BBS2 | CHM | NRL | Gene |
| c.1169T > G | c.1495G > T | c.1495G > T | c.1895G > A | c.1629_16 | c.223dupC | Mutation 1 |
| | | | | 36delGCC | | |
| | | | | AAGAA | | |
| p.(Met390Arg) | p.(Glu499*) | p.(Glu499*) | p.(Arg632Pro) | p.(Lys543Asnfs*21) | p.(Leu75Profs*19) | Mutation 1 |
| | | | | | | Effect |
| CM021489 | CM117343 | CM117343 | CM016198 | This study | CI043431 | HGMD |
| 12118255, | 21642631 | 21642631 | 11567139, | This study | 15459973; | PubMed ID |
| 18032602 | | | 20498079 | | 1733500 | |
| D/P/P | T/NA/P | T/NA/P | D/P/P | NA/NA/P | NA/NA/P | SIFT/PolyPhen/ |
| | | | | | | Mutation |
| | | | | | | Taster |
| G = 26/ | Absent | Absent | G = 1/ | Absent | Absent | EVS |
| T = 12964 | | | C = 12995 | | | Frequency |
| chr11: | chr12: | chr12: | chr16: | -NA- | chr14: | Chromosome |
| 66293652 | 76740270 | 76740270 | 56553703 | | 24551834 | Position |
| c.1169T > G | c.14950 > T | c.1495G > T | c.72C > G | -NA- | c.223dupC | Mutation 2 |
| p.(Met390Arg) | p.(Glu499*) | p.(Glu499*) | p.(Tyr24*) | -NA- | p.(Leu75Profs*19) | Mutation 2 |
| | | | | | | Effect |
| CM021489 | CM117343 | CM117343 | CM016219 | -NA- | CI043431 | HGMD |
| 12118255, | 21642631 | 21642631 | 11567139 | -NA- | 15459973; | PubMed ID |
| 18032602 | | | | | 1733500 | |
| D/P/P | T/NA/P | T/NA/P | D/NA/P | NA | NA/NA/P | PolyPhen/ |
| | | | | | | SIFT/Mutation |
| | | | | | | Taster² |
| G = 26/ | Absent | Absent | C = 1/ | NA | Absent | EVS |
| T = 12964 | | | G = 12993 | | | Frequency |
| Yes | Yes | Yes | Yes | NA | Yes | Segregation |
| | | | | | | Validated |
| OGI-423-901 | OGI-294-638 | OGI-028-068 | OGI-322-765 | OGI-314-749* | OGI-454-958 | Patient ID |
| CRD | CRD | CRD | Ciliopathy | Ciliopathy | Ciliopathy | Clinical |
| | | | | | | Diagnosis¹ |
| D | R | X | R | R | R | Inheritance |
| chr6: | chr2: | chrX: | chr2: | chr8: | chr4: | Chromosomal |
| 42689937 | 112779860 | 46713145 | 73651590 | 94767167 | 122749661 | Position |
| PRPH2 | MERTK | RP2 | ALMS1 | TMEM67 | B9S7 | Gene |
| c.136C > T | c.2375delA | c.337G > A | c.797G > A | c.25G > A | c.1787- | Mutation 1 |
| | | | | | 1G > C | |
| p.(Arg46*) | p.(Ile793*) | p.(Arg113Thr) | p.(Trp266*) | p.(Val9Met) | r.spl | Mutation 1 |
| | | | | | | Effect |
| CM930635 | This study | This study | This study | This study | This study | HGMD |
| 8111389 | This study | This study | This study | This study | This study | PubMed ID |
| T/NA/P | NA/NA/P | D/PosD/P | D/NA/P | D/NA/Poly | splicing | SIFT/PolyPhen/ |
| | | | | | defect | Mutation |
| | | | | | | Taster |
| Absent | Absent | Absent | Absent | A = 33/ | Absent | EVS |
| | | | | G = 12973 | | Frequency |
| -NA- | chr2: | -NA- | chr2: | chr8: | chr4: | Chromosome |
| | 112779859 | | 73677553 | 94794635 | 122782802 | Position |
| -NA- | c.2375delA | -NA- | c.3896C > A | c.1078A > G | c.198T > G | Mutation 2 |
| -NA- | p.(Ile793*) | -NA- | p.(Ser1299*) | p.(Thr360Ala) | p.(Ile66Met) | Mutation 2 |
| | | | | | | Effect |
| -NA- | This study | -NA- | This study | This study | CM102426 | HGMD |
| -NA- | This study | -NA- | This study | This study | 19797195 | PubMed ID |
| -NA- | NA/NA/P | -NA- | D/NA/P | D/P/P | D/PosD/P | PolyPhen/ |
| | | | | | | SIFT/Mutation |
| | | | | | | Taster² |
| -NA- | Absent | -NA- | Absent | G = 28/ | C = 3/ | EVS |
| | | | | A = 12976 | A = 13003 | Frequency |
| Yes | Yes | Yes | Yes | Yes | Yes | Segregation |
| | | | | | | Validated |
| OGI-300-667 | OGI-455-966 | OGI-420-894* | OGI-533-1088 | OGI-300-666 | OGI-064-149 | Patient ID |
| CRD | CRD | CRD | CRD | CRD | CRD | Clinical |
| | | | | | | Diagnosis¹ |
| R | R | R | R | R | R | Inheritance |
| chr4: | chr1: | chr16: | chr1: | chr4: | chr1: | Chromosomal |
| 16010716 | 197298090 | 56530894 | 94486895 | 16010716 | 94463397 | Position |
| PROM1 | CRB1 | BBS2 | ABCA4 | PROM1 | ABCA4 | Gene |
| c.1157T > A | c.613_ | c.1895G > | c.4919G > A | c.1157T > A | c.6729 + 5_ | Mutation 1 |
| | 619delATAGGAA | C | | | 6729 + | |
| | | | | | 19del15 | |
| p.(Leu386*) | p.(Ile205Aspfs*13) | p.(Arg632Pro) | p.(Arg1640Gln) | p.(Leu386*) | | Mutation 1 |
| | | | | | | Effect |
| This Study | CD011132 | CM016198 | CM003577 | This Study | CD107463 | HGMD |
| This study | 11231775, | 11567139, | 10711710; | This study | 20554613, | PubMed ID |
| | 23379534 | 20498079 | 23755871 | | 22264887 | |
| D/NA/P | NA/NA/P | D/D/P | D/D/P | D/NA/P | r.spl | SIFT/PolyPhen/ |
| | | | | | | Mutation |
| | | | | | | Taster |
| Absent | Absent | G = 1/ | Absent | Absent | Absent | EVS |
| | | C = 12995 | | | | Frequency |
| chr4: | chr1: | chr16: | chr1: | chr4: | chr1: | Chromosome |
| 16010716 | 197298090 | 56548399 | 94528132 | 16010716 | 94463397 | Position |
| c.1157T > A | c.613_ | c.311A > C | c.1937 + | c.1157T > A | c.6729 + 5_ | Mutation 2 |
| | 619delATAGGAA | | 1G > A | | 6729 + | |
| | | | | | 19del15 | |
| p.(Leu386*) | p.(Ile205Aspfs*13) | p.(Asp104Ala) | r. spl | p.(Leu386*) | | Mutation 2 |
| | | | | | | Effect |
| This study | CD011132 | CM012901 | CS003487 | This study | CD107463 | HGMD |
| This study | 11231775, | 11567139, | 10958763 | This study | 20554613, | PubMed ID |
| | 23379534 | 20498079 | | | 22264887 | |
| D/NA/P | NA/NA/P | D/P/P | NA/NA/NA | D/NA/P | splicing | PolyPhen/ |
| | | | | | defect | SIFT/Mutation |
| | | | | | | Taster² |
| Absent | Absent | Absent | Absent | Absent | Absent | EVS |
| | | | | | | Frequency |
| Yes | Yes | Yes | NA | Yes | Yes | Segregation |
| | | | | | | Validated |
| OGI-326- | OGI-069- | OGI-079- | OGI-501- | OGI-153- | OGI-310- | Patient ID |
| 773 | 778 | 194 | 1038 | 408* | 725 | |
| LCA | LCA | LCA | FA | FA | CSNB | Clinical |
| | | | | | | Diagnosis¹ |
| R | R | R | R | R | X | Inheritance |
| chr3: | chr1: | chr14: | chr12: | chr1: | chrX: | Chromosomal |
| 121500637 | 10042426 | 21819307 | 56118211 | 197390645 | 41333593 | Position |
| IQCB1 | NMNAT1 | RPGRIP1 | RDH5 | CRB1 | NYX | Gene |
| c.1363C > T | c.507G > A | c.3793_37 | c.839G > A | c.1687A > G | c.887G > T | Mutation 1 |
| | | 94insGAAA | | | | |
| p.(Arg455*) | p.(Trp169*) | p4Val1265Glyfs*8) | p.(Arg280His) | p.(Ser563Gly) | p.(Gly296Val) | Mutation 1 |
| | | | | | | Effect |
| CM110330 | CM127758 | This study | CM993460 | This Study | This study | HGMD |
| 23188109 | 22842231 | This study | 10617778, | This study | This study | PubMed ID |
| | | | 11675386 | | | |
| T/NA/P | NA/NA/P | NA/NA/P | D/PosD/P | D/P/P | D/D/P | SIFT/PolyPhen/ |
| | | | | | | Mutation |
| | | | | | | Taster |
| Absent | A = 2/ | Absent | Absent | Absent | Absent | EVS |
| | G = 13004 | | | | | Frequency |
| chr3: | chr1: | chr14: | chr12: | chr1: | -NA- | Chromosome |
| 121518149 | 10042553 | 21819307 | 56118211 | 197391001 | | Position |
| c.659delG | c.634G > A | c.3793_ | c.839G > A | c.2043T > G | -NA- | Mutation 2 |
| | | 3794insGAAA | | | | |
| p.(Ser220*) | p.(Val212Met) | p.(Val1265Glyfs*18) | p.(Arg280His) | p.(Cys681Trp) | -NA- | Mutation 2 |
| | | | | | | Effect |
| This study | This study | This study | CM993460 | -NA- | -NA- | HGMD |
| This study | This study | This study | 10617778, | 20956273, | -NA- | PubMed ID |
| | | | 11675386 | 11231775 | | |
| NA/NA/P | T/B/Poly | NA/NA/P | D/PosD/P | D/P/P | -NA- | PolyPhen/ |
| | | | | | | SIFT/Mutation |
| | | | | | | Taster² |
| A1 = 1/ | Absent | Absent | Absent | Absent | -NA- | EVS |
| R = 12511 | | | | | | Frequency |
| Yes | Yes | NA | NA | Yes | Yes | Segregation |
| | | | | | | Validated |
| OGI-532- | OGI-523- | OGI-039- | OGI-011- | OGI-010- | OGI-391- | Patient ID |
| 1087 | 1075 | 096 | 033* | 032 | 846 | |
| LCA | LCA | LCA | LCA | LCA | LCA | Clinical |
| | | | | | | Diagnosis¹ |
| R | R | R | R | R | R | Inheritance |
| chr6: | chr1: | chr14: | chr6: | chr12: | chr1: | Chromosomal |
| 80203350 | 10042680 | 88899496 | 42689551 | 88487732 | 68903976 | Position |
| LCA5 | NMNAT1 | SPATA7 | PRPH2 | CEP290 | RPE65 | Gene |
| c.838C > T | c.761A > G | c. 1102_ | c.522C > T | c.3124dupA | c.1022T > C | Mutation 1 |
| | | 1103delCT | | | | |
| p.(Arg280*) | p.(Tyr254Cys) | p.(Leu368Glufs*4) | p.(Trp174*) | p.(Ala1043Glyfs*9) | p.(Leu341Ser) | Mutation 1 |
| | | | | | | Effect |
| This study | This study | This Study | This Study | This Study | CM983763 | HGMD |
| This study | This study | This study | This study | This study | 9501220, | PubMed ID |
| | | | | | 19854499 | |
| T/NA/P | D/P/P | NA/NA/P | NA/NA/P | NA/NA/P | D/P/P | SIFT/PolyPhen/ |
| | | | | | | Mutation |
| | | | | | | Taster |
| Absent | Absent | Absent | Absent | Absent | Absent | EVS |
| | | | | | | Frequency |
| chr6: | chr1: | chr14: | chr6: | chr12: | chr1: | Chromosome |
| 80203350 | 10042688 | 88892690 | 42689551 | 88494960 | 68903976 | Position |
| c.838C > T | c.769G > A | c.487A > T | c.552C > T | c.2991 + | c.1022T > C | Mutation 2 |
| | | | | 1655A > G | | |
| p.(Arg280*) | p.(Glu257Lys) | p.(Lys163*) | p.(Trp174*) | r.spl | p.(Leu341Ser) | Mutation 2 |
| | | | | | | Effect |
| This study | CM127755 | This study | This study | CS064383 | CM983763 | HGMD |
| This study | 22842227; | This study | This study | 16909394 | 9501220, | PubMed ID |
| | 22842231 | | | | 19854499 | |
| T/NA/P | D/P/P | NA/NA/P | NA/NA/P | splicing | D/P/P | PolyPhen/ |
| | | | | defect | | SIFT/Mutation |
| | | | | | | Taster² |
| Absent | A = 14/ | Absent | Absent | -NA- | Absent | EVS |
| | G = 12992 | | | | | Frequency |
| NA | NA | Yes | NA | Yes | NA | Segregation |
| | | | | | | Validated |
| OGI-138- | OGI-315- | OGI-428- | OGI-296- | OGI-520- | OGI-535- | Patient ID |
| 376 | 751 | 910 | 644 | 1072 | 1092 | |
| MD | MD | MD | MD | LCA | LCA | Clinical |
| | | | | | | Diagnosis¹ |
| R | D | R | D | R | R | Inheritance |
| chr11: | chr6: | chr1: | chr6: | chr1: | chr1: | Chromosomal |
| 61719350 | 42689937 | 94495083 | 42689609 | 10035731 | 10032184 | Position |
| BEST1 | PRPH2 | ABCA4 | PRPH2 | NMNAT1 | NMNAT1 | Gene |
| c.72G > A | c.136C > T | c.4457C > T | c.461_ | c.197G > A | c.53A > G | Mutation 1 |
| | | | 463delAGA | | | |
| p.(Trp24*) | p.(Arg46*) | p.(Pro1486Leu) | p.(Lys154del) | p.(Arg66Gln) | p.(Asn18Ser) | Mutation 1 |
| | | | | | | Effect |
| This Study | CM930635 | CM990054 | CD931050 | This study | This study | HGMD |
| This study | 8111389 | 9973280 | 8240110 | -NA- | This study | PubMed ID |
| NA/NA/P | T/NA/P | T/P/P | NA/NA/P | D/P/P | D/P/P | SIFT/PolyPhen/ |
| | | | | | | Mutation |
| | | | | | | Taster |
| Absent | Absent | Absent | Absent | Absent | Absent | EVS |
| | | | | | | Frequency |
| chr11: | -NA- | chr1: | -NA- | chr1: | chr1: | Chromosome |
| 61724418 | | 94495083 | | 10042688 | 10032184 | Position |
| c.584C > T | -NA- | c.4457C > T | -NA- | c.769G > A | c.53A > G | Mutation 2 |
| p.(Ala195Val) | -NA- | p.(Pro1486Leu) | -NA- | p.(Glu257Lys) | p.(Asn18Ser) | Mutation 2 |
| | | | | | | Effect |
| CM004430 | -NA- | CM990054 | -NA- | CM127755 | This study | HGMD |
| 21825197 | -NA- | 9973280 | -NA- | 22842227; | This study | PubMed ID |
| | | | | 22842231 | | |
| D/P/P | -NA- | T/P/P | -NA- | D/P/P | D/P/P | PolyPhen/ |
| | | | | | | SIFT/Mutation |
| | | | | | | Taster² |
| Absent | -NA- | Absent | -NA- | A = 14/ | Absent | EVS |
| | | | | G = 12992 | | Frequency |
| Yes | Yes | Yes | Yes | Yes | Yes | Segregation |
| | | | | | | Validated |
| OGI-051- | OGI-109- | OGI-449- | OGI-286- | OGI-556- | OGI-519- | Patient ID |
| 126 | 279 | 951 | 615 | 1135 | 1068* | |
| RP | OCA | Posterior | MD | MD | MD | Clinical |
| | | Microphthalmos | | | | Diagnosis¹ |
| X | R | R | D | X | R | Inheritance |
| chrX: | chr15: | chr11: | chr6: | chrX: | chr14: | Chromosomal |
| 38178158 | 28116343 | 119212383 | 42672302 | 153420209 | 68195950 | Position |
| RPGR | OCA2 | MFRP | PRPH2 | OPN1LW | RDH12 | Gene |
| c.392_393insGAA | c.2201A > C | c.1615C > T | c.629G > A | c.739C > T | c.701G > A | Mutation 1 |
| p.(His131delinsGlnAsn) | p.(Leu734Arg) | p.(Arg539Cys) | p.(Pro210Leu) | p.(Arg247*) | p.(Arg234His) | Mutation 1 |
| | | | | | | Effect |
| This study | -NA- | This study | CM011806 | CM931121 | CM054833 | HGMD |
| This study | 23103111 | This study | 11139241 | 8213841 | 16269441 | PubMed ID |
| NA/NA/P | D/P/P | D/P/P | D/P/P | T/NA/P | T/B/Poly | SIFT/PolyPhen/ |
| | | | | | | Mutation |
| | | | | | | Taster |
| Absent | Absent | A = 1/ | Absent | Absent | Absent | EVS |
| | | G = 12987 | | | | Frequency |
| -NA- | chr15: | chr11: | -NA- | -NA- | chr14: | Chromosome |
| | 28267662 | 119213687 | | | 68196093 | Position |
| -NA- | c.631C > G | c.1150dupC | -NA- | -NA- | c.844T > G | Mutation 2 |
| -NA- | p.(Pro211Ala) | p.(His384Profs*8) | -NA- | -NA- | p.(Phe282Val) | Mutation 2 |
| | | | | | | Effect |
| -NA- | CM031308 | CI052547 | -NA- | -NA- | This study | HGMD |
| -NA- | 12713581 | 15976030 | -NA- | -NA- | This study | PubMed ID |
| -NA- | D/P/P | NA/NA/P | -NA- | NA | D/P/P | PolyPhen/ |
| | | | | | | SIFT/Mutation |
| | | | | | | Taster² |
| -NA- | Absent | Absent | -NA- | NA | Absent | EVS |
| | | | | | | Frequency |
| NA | Yes | Yes | NA | NA | Yes | Segregation |
| | | | | | | Validated |
| OGI-301- | OGI-301- | OGI-293- | OGI-278- | OGI-140- | OGI-134- | Patient ID |
| 704 | 703 | 636 | 603 | 381 | 315 | |
| RP | RP | RP | RP | RP | RP | Clinical |
| | | | | | | Diagnosis¹ |
| D | D# | D | R | R | X | Inheritance |
| chr6: | chr6: | chr3: | chr1: | chr4: | chrX: | Chromosomal |
| 42672300 | 42672300 | 129252535 | 215848679 | 187117196 | 38178080 | Position |
| PRPH2 | PRPH2 | RHO | USH2A | CYP4V2 | RPGR | Gene |
| c.631T > C | c.631T > C | c.1021G > A | c.12574G > | c.367A > G | c.469 + 2T > | Mutation 1 |
| | | | A | | A | |
| p.(Phe211Leu) | p.(Phe211Leu) | p.(Glu341Lys) | p.(Arg4192Cys) | p.(Met123Val) | -NA- | Mutation 1 |
| | | | | | | Effect |
| CM951119 | CM951119 | CM973327 | This study | CM041498 | CS1212410 | HGMD |
| 9673478 | 9673478 | 21094163 | This study | 15042513 | 23150612 | PubMed ID |
| D/P/P | D/P/P | T/PosD/P | D/P/Poly | T/B/P | | SIFT/PolyPhen/ |
| | | | | | | Mutation |
| | | | | | | Taster |
| Absent | Absent | A = 1/ | Absent | Absent | Absent | EVS |
| | | G = 13005 | | | | Frequency |
| -NA- | -NA- | -NA- | chr1: | chr4: | -NA- | Chromosome |
| | | | 215848679 | 187117196 | | Position |
| -NA- | -NA- | -NA- | c.12574G > | c.367A > G | -NA- | Mutation 2 |
| | | | A | | | |
| -NA- | -NA- | -NA- | p.(Arg4192Cys) | p.(Met123Val) | -NA- | Mutation 2 |
| | | | | | | Effect |
| -NA- | -NA- | -NA- | This study | CM041498 | -NA- | HGMD |
| -NA- | -NA- | -NA- | This study | 15042513 | -NA- | PubMed ID |
| -NA- | -NA- | -NA- | D/P/Poly | T/B/P | -NA- | PolyPhen/ |
| | | | | | | SIFT/Mutation |
| | | | | | | Taster² |
| -NA- | -NA- | -NA- | Absent | Absent | -NA- | EVS |
| | | | | | | Frequency |
| Yes | Yes | Yes | Yes | Yes | NA | Segregation |
| | | | | | | Validated |
| OGI-411- | OGI-406- | OGI-405- | OGI-388- | OGI-323- | OGI-311- | Patient ID |
| 879 | 874 | 871 | 841 | 766 | 746 | |
| RP | RP | RP | RP | RP | RP | Clinical |
| | | | | | | Diagnosis¹ |
| R | R | R | R | R | R | Inheritance |
| chr1: | chr1: | chr1: | chr1: | chr6: | chr3: | Chromosomal |
| 197390396 | 216498866 | 197396961 | 215972325 | 64694275 | 129251616 | Position |
| CRB1 | USH2A | CRB1 | USH2A | EYS | RHO | Gene |
| c.1438T > C | c.920_ | c.2506C > A | c.9882C > | c.7055 + 1G > | c.936 + 1G > | Mutation 1 |
| | 923dup | | G | T | T | |
| p.(Cys480Arg) | p.(His308Glnfs*15) | p.(Pro836Thr) | p.(Cys3294Trp) | -NA- | r. spl? | Mutation 1 |
| | | | | | | Effect |
| CM010800 | -NA- | CM043271 | This study | This study | CS920776 | HGMD |
| 11231775 | 15325563, | 20956273, | This study | This study | 21357407 | PubMed ID |
| | 10729113, | 15459956 | | | | |
| | 22135276, | | | | | |
| | 11311042 | | | | | |
| D/P/P | NA/NA/P | D/P/Poly | D/P/P | r.spl | NA/NA/NA | SIFT/PolyPhen/ |
| | | | | | | Mutation |
| | | | | | | Taster |
| Absent | A1 = 4/R = | A = 12/C = | Absent | Absent | Absent | EVS |
| | 12516 | 12994 | | | | Frequency |
| chr1: | chr1: | chr1: | chr1: | chr6: | chr3: | Chromosome |
| 197396961 | 216420460 | 197396961 | 216495263 | 64694275 | 129251616 | Position |
| c.2506C > A | c.2276G > T | c.2506C > A | c.1606T > C | c.7055 + | c.936 + | Mutation 2 |
| | | | | 1G > T | 1G > T | |
| p.(Pro836Thr) | p.(Cys759Phe) | p.(Pro836Thr) | p.(Cys536Arg) | -NA- | r. spl | Mutation 2 |
| | | | | | | Effect |
| CM043271 | CM001372 | CM043271 | CM001807 | This study | CS920776 | HGMD |
| 20956273, | 10775529, | 20956273, | 10909849, | This study | 21357407 | PubMed ID |
| 15459956 | 18281613, | 15459956 | 15043528 | | | |
| | [symbol] | | | | | |
| D/P/P | D/P/P | D/P/Poly | D/P/P | splicing | splicing | PolyPhen/ |
| | | | | defect | defect | SIFT/Mutation |
| | | | | | | Taster² |
| A = 12/ | A = 20/ | A = 12/ | Absent | Absent | Absent | EVS |
| C = 12994 | C = 12986 | C = 12994 | | | | Frequency |
| Yes | NA | Yes | Yes | Yes | Yes | Segregation |
| | | | | | | Validated |
| OGI-059- | OGI-OGI- | OGI-021- | OGI-006- | OGI-546- | OGI-390- | Patient ID |
| 141 | 075 | 053 | 012 | 1114 | 845 | |
| RP | RP | RP | RP | RP | RP | Clinical |
| | | | | | | Diagnosis¹ |
| D | R | R | X | R | D | Inheritance |
| chr2: | chr6: | chr1: | chrX: | chr1: | chr3: | Chromosomal |
| 96953706 | 35467809 | 216219858 | 46713066 | 215960057 | 129252547 | Position |
| SNRNP200 | TULP1 | USH2A | RP2 | USH2A | RHO | Gene |
| c.3260C > T | c.1444C > T | c.6240G > T | c.258T > G | c.10342G > A | c.1033G > A | Mutation 1 |
| p.(Ser1087Leu) | p.(Arg482Trp) | p.(Lys2080Asn) | p.(Cys86Trp) | p.(Glu3448Lys) | p.(Val345Met) | Mutation 1 |
| | | | | | | Effect |
| CM097780 | CM073391 | -NA- | -NA- | -NA- | CM910347 | HGMD |
| 19878916 | 17620573 | 20507924 | 10937588; | 24265693 | 1833777 | PubMed ID |
| | | | 21738648 | | | |
| D/NA/P | D/P/P | T/PosD/Poly | D/P/P | T/PosD/P | D/B/P | SIFT/PolyPhen/ |
| | | | | | | Mutation |
| | | | | | | Taster |
| Absent | Absent | A = 130/ | Absent | T = 1/ | Absent | EVS |
| | | C = 12876 | | C = 13005 | | Frequency |
| -NA- | chr6: | ch1: | -NA- | chr1: | -NA- | Chromosome |
| | 35473823 | 216246606 | | 216166497 | | Position |
| -NA- | c.956G > A | c.5609G > A | -NA- | c.6670G > T | -NA- | Mutation 2 |
| -NA- | p.(Gly319Asp) | p.(Arg1870Gln) | -NA- | p.(Gly2224Cys) | -NA- | Mutation 2 |
| | | | | | | Effect |
| -NA- | This study | This study | -NA- | This study | -NA- | HGMD |
| -NA- | This study | This study | -NA- | This study | -NA- | PubMed ID |
| -NA- | D/P/P | T/P/P | -NA- | D/P/P | -NA- | PolyPhen/ |
| | | | | | | SIFT/Mutation |
| | | | | | | Taster2 |
| -NA- | Absent | T = 3/ | -NA- | A = 4/ | -NA- | EVS |
| | | C = 13003 | | C = 13002 | | Frequency |
| NA | NA | Yes | Yes | Yes | NA | Segregation |
| | | | | | | Validated |
| OGI-274- | OGI-151-405 | OGI-149- | OGI-144- | OGI-098- | OGI-088- | Patient ID |
| 582 | | 401 | 390 | 246 | 220 | |
| RP | RP | RP | RP | RP | RP | Clinical |
| | | | | | | Diagnosis¹ |
| D# | R | R | R | R | D | Inheritance |
| chr7: | chr2: | chr1: | chr1: | chr8: | chr4: | Chromosomal |
| 128038580 | 62066572 | 197396584 | 215848678 | 96264450 | 16077349 | Position |
| IMPDH1 | FAM161A | CRB1 | USH2A | C8orf37 | PROM1 | Gene |
| c.962C > T | c.1567C > T | c.2129A > T | c.12575G > | c.436A > C | c.181A > G | Mutation 1 |
| | | | A | | | |
| p.(Ala321Val) | p.(Arg523*) | p.(Glu710Val) | p.(Arg4192His) | p.(Met146Leu) | p.(Ile61Val) | Mutation 1 |
| | | | | | | Effect |
| This Study | CM105642 | CM107849 | CM108280 | This Study | This Study | HGMD |
| This study | 20705279 | 20956273, | 21151602, | This study | This study | PubMed ID |
| | | 20591486 | 22135276 | | | |
| D/P/P | T/NA/P | D/P/P | T/B/Poly | T/PosD/Poly | T/NA/Poly | SIFT/PolyPhen/ |
| | | | | | | Mutation |
| | | | | | | Taster |
| Absent | [symbol] | T = 1/ | T = 2/ | G = 10/ | C = 1/ | EVS |
| | variant | A = 13005 | C = 13004 | T = 12996 | T = 12065 | Frequency |
| | reported at this | | | | | |
| | position | | | | | |
| | c.1567C > G at | | | | | |
| -NA- | chr2: | chr1: | chr1: | chr8: | -NA- | Chromosome |
| | 62066830 | 197396745 | 215848678 | 96264494 | | Position |
| -NA- | c.1309A > T | c.22900 > T | c.12575G > | c.392G > A | -NA- | Mutation 2 |
| | | | A | | | |
| -NA- | p.(Ar9437*) | p.(Arg764Cys) | p.(Arg4192His) | p.(Arg131His) | -NA- | Mutation 2 |
| | | | | | | Effect |
| -NA- | CM105648 | CM992151 | CM108280 | This study | -NA- | HGMD |
| -NA- | 20705278 | 20956273, | 21151602, | This study | -NA- | PubMed ID |
| | | 10508521 | 22115276 | | | |
| -NA- | T/NA/P | T/B/P | T/B/Poly | D/P/P | -NA- | PolyPhen/ |
| | | | | | | SIFT/Mutation |
| | | | | | | Taster² |
| -NA- | A = 5/ | T = 3/ | T = 2/ | T = 10/ | -NA- | EVS |
| | T = 11975 | C = 13003 | C = 13004 | C = 12996 | | Frequency |
| Yes | Yes | Yes | NA | Yes | Yes | Segregation |
| | | | | | | Validated |
| OGI-555- | OGI-309- | OGI-529- | OGI-570- | OGI-513- | OGI-513- | Patient ID |
| 1134 | 723 | 1084 | 1170 | 1054 | 1053 | |
| RP | RP | RP | RP | RP | RP | Clinical |
| | | | | | | Diagnosis¹ |
| R | R | R | D | R | R | Inheritance |
| chr6: | ch11: | chr5: | chr17: | ch1: | ch1: | Chromosomal |
| 64709008 | 66293652 | 149274769 | 1558827 | 197325970 | 197325970 | Position |
| EYS | BBS1 | PDE6A | PRPF6 | CRB1 | CRB1 | Gene |
| c.6794delC | c.1169T > G | c.1705C > A | c.5804G > A | c.998G > C | c.998G > C | Mutation 1 |
| p.(Pro2265Glnfs*46) | p.(Met390Arg) | pQ569K | p.(Arg1935His) | p.(Gly333Ala) | p.(Gly333Ala) | Mutation 1 |
| | | | | | | Effect |
| CD102743 | CM021489 | CM994743 | This study | CM082583 | CM082583 | HGMD |
| 20333770 | 12118255, | 10393062 | This study | 18682808 | 18682808 | PubMed ID |
| | 18032602 | | | | | |
| NA/NA/P | D/P/P | D/P/P | NA/P/P | D/P/P | D/P/P | SIFT/PolyPhen/ |
| | | | | | | Mutation |
| | | | | | | Taster |
| A1 = 10/ | G = 26/ | T = 2/ | Absent | Absent | Absent | EVS |
| R = 6664 | T = 12964 | G = 13004 | | | | Frequency |
| chr6: | ch11: | chr5: | -NA- | chr1: | chr1: | Chromosome |
| 65300430 | 66293652 | 149277996 | | 197396856 | 197396856 | Position |
| c.5330T > A | c.1169T > G | c.1336delA | -NA- | c.2401A > T | c.2401A > T | Mutation 2 |
| p.(Leu1777*) | p.(Met390Arg) | p.(Arg446Glyfs*8) | -NA- | p.(Lys801*) | p.(Lys801*) | Mutation 2 |
| | | | | | | Effect |
| This study | CM021489 | This study | -NA- | CM012914 | CM012914 | HGMD |
| This study | 12118255, | This study | -NA- | 20956273, | 20956273, | PubMed ID |
| | 18032602 | | | 11389483 | 11389483 | |
| NA/NA/P | D/P/P | NA/NA/P | NA | T/NA/P | T/NA/P | PolyPhen/ |
| | | | | | | SIFT/Mutation |
| | | | | | | Taster² |
| Absent | G = 26/ | Absent | NA | T = 1/ | T = 1/ | EVS |
| | T = 12964 | | | A = 13003 | A = 13003 | Frequency |
| Yes | Yes | NA | Yes | Yes | Yes | Segregation |
| | | | | | | Validated |
| OGI-261- | OGI-154- | OGI-289- | OGI-319- | OGI-303- | OGI-276- | Patient ID |
| 547 | 590 | 627 | 764 | 707 | 589 | |
| Star | Star | RS | RP | RP | RP | Clinical |
| | | | | | | Diagnosis¹ |
| R | R | X | X | X | R | Inheritance |
| chr1: | chr1: | chrX: | chrX: | chrX: | chr16: | Chromosomal |
| 94508969; | 94467475 | 18660162 | 38158220 | 46719451 | 57938764 | Position |
| chr1: | | | | | | |
| 94528806 | | | | | | |
| ABCA4 | ABCA4 | RS1 | RPGR | RP2 | CNGB1 | Gene |
| c.[3113G > | c.6221G > A | c.637C > T | c.1234C > T | c.797A > C | c.2508G > T | Mutation 1 |
| A; | | | | | | |
| 622A > G] | | | | | | |
| p.[(Ala1038Val; | p.(Gly2074Asp) | p.(Arg213Trp) | p.(Arg412*) | p.(Gln266Pro) | p.(Tyr836*) | Mutation 1 |
| Leu541Pro)] | | | | | | Effect |
| CM970006; | This Study | CM981774 | CM021145 | This study | This Study | HGMD |
| CM990022 | | | | | | |
| 16103129, | This study | 9618178, | 11992260 | This study | This study | PubMed ID |
| 9781034, | | 12417531 | | | | |
| 19074458 | | | | | | |
| T/P/P; | D/D/P | D/P/P | D/PosD/P | D/D/P | NA/NA/P | SIFT/PolyPhen/ |
| D/P/P | | | | | | Mutation |
| | | | | | | Taster |
| A = 22/ | T = 1/ | Absent | Absent | Absent | Absent | EVS |
| G = 12984; | C = 13005 | | | | | Frequency |
| G = 2/ | | | | | | |
| A = 13004 | | | | | | |
| chr1: | chr1: | -NA- | -NA- | -NA- | chr16: | Chromosome |
| 94512496 | 94543290 | | | | 57949173 | Position |
| c.2897C > T | c.1510A > C | -NA- | -NA- | -NA- | c.2284G > A | Mutation 2 |
| p.(Gly966Glu) | p.(Asn504His) | -NA- | -NA- | -NA- | p.(Arg762Cys) | Mutation 2 |
| | | | | | | Effect |
| This study | This study | -NA- | -NA- | -NA- | CM118405 | HGMD |
| This study | This study | -NA- | -NA- | -NA- | 21987686 | PubMed ID |
| D/P/P | T/B/P | -NA- | -NA- | -NA- | D/P/P | PolyPhen/SIFT/ |
| | | | | | | Mutation |
| | | | | | | Taster² |
| Absent | Absent | -NA- | Absent | Absent | Absent | EVS |
| | | | | | | Frequency |
| Yes | Yes | NA | Yes | Yes | Yes | Segregation |
| | | | | | | Validated |
| OGI-146- | OGI-435- | OGI-424- | OGI-312- | OGI-304- | OGI-288- | Patient ID |
| 393 | 928 | 903 | 735 | 709 | 602 | |
| Star | Star | Star | Star | Star | Star | Clinical |
| | | | | | | Diagnosis¹ |
| R | R | R | R | R | R | Inheritance |
| chr1: | chr1: | chr1: | chr1: | chr1: | chr1: | Chromosomal |
| 94467548 | 94510248 | 94473807 | 94490567 | 94463583; | 94473807; | Position |
| | | | | chr1: | chr1: | |
| | | | | 94496053 | 94496666 | |
| ABCA4 | ABCA4 | ABCA4 | ABCA4 | ABCA4 | ABCA4 | Gene |
| c.6148G > | c.2971G > | c.5882G > A | c.4577C > T | c.[6563T > | c.[5882G > | Mutation 1 |
| C | C | | | C; | A; | |
| | | | | 4283C > T] | 4139C > T] | |
| p.(Val2050Leu) | p.(Gly991Arg) | p.(Gly1961Glu) | p.(Thr1562Met) | p.[(Phe2188Ser; | p.[(Gly1961Glu; | Mutation 1 |
| | | | | Thr1428Met)] | Pro1380Leu)] | Effect |
| CM970020 | CM032807 | CM970016 | CM990057 | CM023878; | CM970016; | HGMD |
| | | | | Novel | CM990044 | |
| 11328725 | 14517951 | 11017087, | 9973280, | 12202497; | 11017087, | PubMed ID |
| | | 19074458 | 11017087 | 11818392 | 19074458; | |
| | | | | | 9973280, | |
| | | | | | 15579991 | |
| D/P/P | D/P/P | D/P/P | D/P/P | D/P/P; | D/P/P; | SIFT/PolyPhen/ |
| | | | | T/B/Poly | D/P/P | Mutation |
| | | | | | | Taster |
| G = 48/ | G = 28/ | T = 41/ | Absent | Absent; | T = 41/ | EVS |
| C = 12958 | C = 12978 | C = 12965 | | A = 2/ | C = 12965; | Frequency |
| | | | | G = 13004 | A = 2/ | |
| | | | | | G = 13004 | |
| chr1: | chr1: | ch1: | chr1: | chr1: | chr1: | Chromosome |
| 94517254 | 99454409 | 94476951 | 94508353 | 94578582 | 94520705 | Position |
| c.2588G > | c.1208A > T | c.5461- | c.3292C > T | c.101_ | c.2549A > G | Mutation 2 |
| C | | 10T > C | | 106delCTTTAT | | |
| p.(Gly863Ala) | p.(Asp403Val) | r.spl | p.(Arg1098Cys) | p.(Ser34_ | p.(Tyr850Cys) | Mutation 2 |
| | | | | Leu35del) | | Effect |
| -NA- | This study | CS057513 | CM003377 | This study | CM129869 | HGMD |
| 11017087, | This study | 15614537, | 19074458 | This study | 23096905 | PubMed ID |
| 9054934, | | 10958763 | | | | |
| [symbol] | | | | | | |
| D/P/P | D/P/P | splicing | D/P/P | NA/NA/P | D/P/P | PolyPhen/ |
| | | defect | | | | SIFT/Mutation |
| | | | | | | Taster² |
| G = 68/ | Absent | G = 3/ | Absent | Absent | C = 1/ | EVS |
| C = 1 [symbol] | | A = 13003 | | | T = 13005 | Frequency |
| Yes | NA | NA | Yes | Yes | Yes | Segregation |
| | | | | | | Validated |
| OGI-518- | OGI-317- | OGI-101- | OGI-549- | OGI-389- | OGI-022- | Patient ID |
| 1066 | 754 | 253 | 1123 | 844 | 055 | |
| U | U | Star | Star | Star | Star | Clinical |
| | | | | | | Diagnosis¹ |
| R | R | R | R | R | R | Inheritance |
| chr11: | chr1: | chr1: | ch1: | chr1: | chr1: | Chromosomal |
| 17547977 | 215812532; | 94476951 | 94473807 | 94471025 | 94476951 | Position |
| | chr1: | | | | | |
| | 216061803 | | | | | |
| USH1C | USH2A | ABCA4 | ABCA4 | ABCA4 | ABCA4 | Gene |
| c.590delG | c.[15017C > | c.5461- | c.5882G > A | c.6119G > A | c.5461- | Mutation 1 |
| | A; | 10T > C | | | 10T > C | |
| | c.81880 > T | | | | | |
| | [symbol] | | | | | |
| p.(Gly197A | p.[(Thr500 | r. spl | p.(Gly1961 | p.(Arg2040 | r. spl | Mutation 1 |
| lafs*21) | 6Lys; | | Glu) | Gln) | | Effect |
| | Pro2730Ser)] | | | | | |
| This study | This study | CS057513 | CM970016 | CM124700 | CS057513 | HGMD |
| This study | This study | 15614537, | 11017087, | 22449572 | 15614537, | PubMed ID |
| | | 10958763 | 19074458 | | 10958763 | |
| NA/NA/P | T/P/P; | splicing | D/P/P | D/P/P | splicing | SIFT/PolyPhen/ |
| | D/D/P | defect | | | defect | Mutation |
| | | | | | | Taster |
| Absent | Absent | G = 3/ | T = 41/ | T = 3/ | G = 3/ | EVS |
| | | A = 13003 | C = 12965 | C = 13003 | A = 13003 | Frequency |
| chr11: | chr1: | chr1: | chr1: | chr1: | chr1: | Chromosome |
| 17547977 | 216495251 | 94473807 | 94495177 | 94476951 | 94485137 | Position |
| c.590delG | c.1618C > T | c.5882G > A | c.4363T > C | c.5461- | c.5196 + | Mutation 2 |
| | | | | 10T > C | 1G > A | |
| p.(Gly197Alafs*21) | p.(Gln540*) | p.(Gly1961Glu) | p.(Cys1455Arg) | r. spl | r. spl | Mutation 2 |
| | | | | | | Effect |
| This study | This study | CM970016 | CM074657 | CS057513 | CS124701 | HGMD |
| This study | This study | 11017087, | 22449572, | 15614537, | 9666097 | PubMed ID |
| | | 19074458 | 17982420 | 10958763 | | |
| NA/NA/P | T/NA/P | D/P/P | D/P/P | splicing | splicing | PolyPhen/ |
| | | | | defect | defect | SIFT/Mutation |
| | | | | | | Taster² |
| Absent | Absent | T = 41/ | Absent | G = 3/ | Absent | EVS |
| | | C = 12965 | | A = 13003 | | Frequency |
| Yes | Yes | Yes | Yes | NA | Yes | Segregation |
| | | | | | | Validated |

| | | |
|---|---|---|
| OGI-277- | OGI-547- | Patient ID |
| 591 | 1117 | |
| U | U | Clinical |
| | | Diagnosis¹ |
| R | R | Inheritance |
| chr5: | chr7: | Chromosomal |
| 89923307 | 92130876 | Position |
| GPR98 | PEX1 | Gene |
| c.956dup | c.2528G > A | Mutation 1 |
| p.(Asn319Lysfs*6) | p.(Gly843Asp) | Mutation 1 |
| | | Effect |
| This Study | CM971144 | HGMD |
| This study | 9398847 | PubMed ID |
| NA/NA/P | D/P/P | SIFT/PolyPhen/ |
| | | Mutation |
| | | Taster |
| Absent | T = 8/ | EVS |
| | C = 12998 | Frequency |
| chr5: | chr7: | Chromosome |
| 90151630 | 92130876 | Position |
| c.17668_17669delAT | c.2528G > A | Mutation 2 |
| p.(Met5890Valfs*10) | p.(Gly843Asp) | Mutation 2 |
| | | Effect |
| CD112917 | CM971144 | HGMD |
| 21569298, | 9398847 | PubMed ID |
| 27147858 | | |
| NA/NA/P | D/P/P | PolyPhen/ |
| | | SIFT/Mutation |
| | | Taster² |
| A1 = 6/ | T = 8/ | EVS |
| R = 11726 | C = 12998 | Frequency |
| Yes | Yes | Segregation |
| | | Validated |

¹Best-Best Disease; CD-Cone Dystrophy; CRD-Cone-Rod Dystrophy; ChorD-Chorioretinal Dystrophy; Choroideremia-Choroideremia; Ciliopathy-Ciliopathy; CSNB-Congenital Stationary Night Blindness; LCA-Leber Congenital Amaurosis; OCA-Oculocutaneous Albinism; MD-Macular Dystrophy; Microphthalmos-Microphthalmos; RP-Retinitis Pigmentosa; RPunc-Retinitis Punctata; RS-juvenile retinoschisis; Star-Stargardt Disease; U-Usher Syndrome.
²SIFT: D = Damaging, T = Tolerated, NA-no prediction. PolyPhen: D = Probably Damaging, PosD = Possibly Damaging, B = Benign, NA = No prediction. Mutation Taster: P = Predicted to be Disease Causing, Poly = Polymorphism, NA = No prediction
*Non-classical genotype-phenotype correlation
# de novo mutation
NA, samples not available for segregation analyses [symbol]
indicates data missing or illegible when filed

TABLE 8
No diagnosis from GEDi testing.

| Subject ID | Clinical Diagnosis¹ | Clinical genetic testing |
|---|---|---|
| OGI-036-091 | CD | Yes |
| OGI-561-1151 | CD | No |
| OGI-283-610 | ChorD | No |
| OGI-292-633 | ChroD | No |
| OGI-275-585 | Ciliopathy | No |
| OGI-290-628 | Ciliopathy | No |
| OGI-298-653 | Ciliopathy | No |
| OGI-037-092 | Ciliopathy | No |
| OGI-410-878 | CRD | No |
| OGI-137-374 | CRD | No |
| OGI-266-572 | CRD | No |
| OGI-400-862 | CRD | No |
| OGI-068-168 | LCA | Yes |
| OGI-080-195 | LCA | No |
| OGI-082-201 | LCA | No |
| OGI-281-608 | LCA | No |
| OGI-299-655 | LCA | No |
| OGI-299-656 | LCA | No |
| OGI-081-197 | LCA | Yes |
| OGI-402-864 | LCA | No |
| OGI-421-895 | LCA | No |
| OGI-306-716 | MD | Yes |
| OGI-429-913 | MD | No |
| OGI-438-932 | MD | No |
| OGI-525-1079 | MD | No |
| OGI-318-755 | MD | No |
| OGI-458-975 | MD | No |
| OGI-395-885 | MD | No |
| OGI-393-853 | PPCRA | No |
| OGI-511-1051 | RD | No |
| OGI-495-1019 | RD | No |
| OGI-496-1020 | RD + brain malformation | No |
| OGI-090-223 | RP | Yes |
| OGI-086-213 | RP | Yes |
| OGI-132-354 | RP | No |
| OGI-282-609 | RP | No |
| OGI-284-611 | RP | No |
| OGI-302-706 | RP | No |
| OGI-313-747 | RP | No |
| OGI-051-126 | RP | Yes |
| OGI-295-640 | RP | No |
| OGI-143-388 | RP | Yes |
| OGI-015-082 | RP | Yes |
| OGI-001-001 | RP | Yes |
| OGI-004-009 | RP | Yes |
| OGI-012-035 | RP | Yes |
| OGI-014-038 | RP | Yes |
| OGI-016-043 | RP | Yes |
| OGI-279-604 | RP | No |
| OGI-412-881 | RP | No |
| OGI-419-891 | RP | No |
| OGI-425-904 | RP | No |
| OGI-315-751 | RP | No |
| OGI-316-752 | RP | No |
| OGI-401-863 | RP | No |
| OGI-105-226 | RP | No |
| OGI-436-929 | RP | No |
| OGI-437-931 | RP | No |
| OGI-291-632 | RP | No |
| OGI-267-573 | RP | No |
| OGI-414-883 | RP | No |
| OGI-320-759 | RP | No |
| OGI-397-857 | RP | No |
| OGI-404-870 | RP | No |
| OGI-457-968 | RP | No |
| OGI-500-1030 | RP | No |
| OGI-498-1026 | RP | No |
| OGI-499-1027 | RP | Yes |
| OGI-504-1041 | RP | No |
| OGI-505-1042 | RP | No |
| OGI-506-1043 | RP | No |
| OGI-508-1047 | RP | No |
| OGI-510-1050 | RP | No |
| OGI-507-1046 | RP | No |
| OGI-514-1057 | RP | No |
| OGI-040-100 | RP | Yes |
| OGI-521-1073 | RP | No |
| OGI-524-1078 | RP | No |
| OGI-527-1082 | RP | No |
| OGI-528-1083 | RP | No |
| OGI-018-081 | RP | Yes |
| OGI-536-1098 | RP | No |
| OGI-497-1021 | RP | No |
| OGI-325-772 | RP | No |
| OGI-152-407 | Star | Yes |
| OGI-091-226 | Star | Yes |
| OGI-044-109 | Star | Yes |
| OGI-271-579 | Star | No |
| OGI-287-617 | Star | No |

¹CD—Cone Dystrophy;
CRD—Cone-Rod Dystrophy;
ChorD—Chorioretinal Dystrophy;
Ciliopathy—ciliopathy;
J—Joubert syndrome;
LCA—Leber's Congenital Amaurosis;
MD—Macular Dystrophy;
PPCRA—pigmented paravenous chorioretinal atrophy,
RD—retinal dystrophy;
RP—Retinitis Pigmentosa;
Star—Stargardt Disease

### Example 9. Mutation Validation

In total, we identified 147 likely pathogenic mutations by GEDi capture and NGS sequencing, and all but four of these were validated by PCR and Sanger sequencing (Supplemental methods and Table 9). Review of the NGS data for the 4 putative mutations that were not validated by Sanger sequencing showed that 3 of the 4 mutations, corresponding to 2 probands (OGI-040-100 and OGI-271-579) were detected by less than 10 reads (Table 9). The 4^{th} putative mutation not detected by Sanger sequencing, had excellent DoC (Table 9); however, the heterozygous G base call was due to mis-alignment of some of the NGS sequence reads, resulting in a false positive variant call (FIG. 2B).

TABLE 9
GEDi mutations not validated by Sanger sequencing

| | | | | | Read | | | | |
|---|---|---|---|---|---|---|---|---|---|
| | | Chromo- | | | Depth | | Chromo- | | |
| Subject | Clinical | somal | | Muta- | (variant/ | Mutation 1 | somal | Mutation | Mutation |
| ID | Diagnosis | Position | Gene | tion 1 | stotal) | Effect | Position | 2 | 2 Effect |
| OGI- | RP | chr17: | A1PL1 | c.1061 | 6/13 | p.(Pro354Arg) | chr17: | c.1060C > | p.(Pro354Thr) |
| 040- | | 6328874 | | C > G | | | 6328875 | A | |
| 100 | | | | | | | | | |
| OGI- | RP | chr17: | FSCN2 | c.1028 | 14/32 | p.(Val343Gly) | -NA- | -NA- | -NA- |
| 267- | | 79503216 | | T > G | | | | | |
| 573 | | | | | | | | | |
| OGI- | Star | chr20: | PRPF6 | c.1094 | 4/14 | p.(Ala365Asp) | -NA- | -NA- | -NA- |
| 271- | | 62632500 | | C > A | | | | | |
| 579 | | | | | | | | | |

### Example 10. Missed Diagnoses

GEDi capture and sequencing did not initially identify a genetic cause of disease in 5 patients for whom genetic diagnoses were ultimately obtained (Table 10). These cases are instructive, and information from them has been used to iteratively improve the GEDi test. For example, in two cases, OGI-147-394 and OGI-387-839, GEDi sequencing identified a single potentially pathogenic variant in ABCA4 and USH2A, respectively, but the second mutant allele was not initially detected (Table 10). The second alleles were subsequently identified by Sanger sequencing, both being deep intronic mutations known to alter splicing^{31,33}. Probes for the relevant intronic region for USH2A have been added to subsequent versions of the GEDi capture set (Table 1b), and those corresponding to deep intronic ABCA4 mutations will be added to the next version of GEDi³³.

One subject with presumed X-linked inheritance of RP was not diagnosed using the GEDi capture and sequencing approach. Since the ORF15 region of RPGR, which is known to harbor many of the mutations that cause RP, is highly repetitive and thus cannot be captured using targeted enrichment methods, we employed PCR amplification of the ORF15 region followed by Sanger sequencing for this patient¹⁰. This approach identified a mutation in ORF15 of RPGR in OGI-089-222 (Table 10).

Probes for the NMNAT1 gene were not included in the original version of the GEDi capture set used for a portion of these studies, resulting in a missed diagnosis for subject OGI-007-017. Exome sequencing for this subject and family resulted in identification of NMNAT1 as an LCA disease gene and probes for this gene are now included in the GEDi capture set (Table 10)³. A similar approach was used for subject OGI-032-076, who was identified to have mutations in the PPT1 gene via exome sequencing. This male subject presented at age 8 with a history of decreased vision and photophobia since age 5, and was diagnosed with cone-rod degeneration based on exam and ERG findings. The subject met all developmental milestones at appropriate ages and there was no evidence of regression. GEDi capture and sequencing did not identify mutations in any of the known IRD disease genes. Research-based exome sequencing of the proband, his parents, and two unaffected brothers identified two rare, potentially pathogenic variants in the PPT1 gene, encoding palmitoyl-protein thioesterase-1, mutations in which cause neuronal ceroid lipofuscinosis-1 (CLN1:MIM 256730). One of these variants p.(Arg151Ter) is the most common nonsense mutation reported in PPT1, and is associated with severe disease in the homozygous state¹¹. The second variant, p.(Met1Leu), had not been previously reported. Measurements of the palmitoyl-protein thioesterase activity in white blood cells from the subject showed reduced enzyme activity, at 8.15 nM/punch*hr (normal range 29.34 to 180.64 nM/punch*hr)^{9,12}. Since the p.Arg151Ter allele is known not to produce a protein, this activity is attributed to the p.Met1Leu protein. Three years after presentation, this subject continues to do well neurologically, possibly consistent with this limited residual PPT1 enzyme activity, although his prognosis remains uncertain^{13,14} . Probes for PPT1 and other genes associated with neuronal ceroid lipofuscinosis are now part of the GEDi capture set (Table 1a).

TABLE 10
Subjects with genetic diagnoses initially missed by GEDi testing.
All the reported mutations were validated and showed familial segregation by Sanger
sequencing. Clinical diagnoses: CRD, Cone-Rod Dystrophy; LCA, Leber Congenital
Amaurosis; RP, Retinitis Pigmentosa; Star, Stargardt Disease; U, Usher syndrome.

| Subject | Clinical | Gene | RefSeq | | | PubMed |
|---|---|---|---|---|---|---|
| ID | Diagnosis | Symbol | Nucleotide ID | Mutation 1 | Mutation 1 Effect | ID |
| OGI- | CRD | PPT1 | NM_000310.3 | c.451C > T | p.(Arg151*) | 9425237 |
| 032- | | | | | | |
| 076 | | | | | | |
| OGI- | LCA | NMNAT1 | NM_022787.3 | c.196C > T | p.(Arg66Trp) | 22842227 |
| 007- | | | | | | |
| 017 | | | | | | |
| OGI- | RP | RPGR | NM_001034853.1 | c.2714_2715delAA | p.(Glu905Glyfs*173) | 14564670 |
| 089- | | | | | | |
| 222 | | | | | | |
| OGI- | Star | ABCA4 | NM_000350.2 | c.5461-10T > C | splicing defect | 10958763 |
| 147- | | | | | | |
| 394 | | | | | | |
| OGI- | U | USH2A | NM_206933.2 | c.2299delG | p.(Glu767Serfs*21) | 11402400 |
| 387- | | | | | | |
| 839 | | | | | | |
| Subject | Clinical | Gene | RefSeq | | | PubMed |
| ID | Diagnosis | Symbol | Nucleotide ID | Mutation 2 | Mutation 2 Effect | ID |
| OGI- | CRD | PPT1 | NM_000310.3 | c.1A > C | p.(Met1Leu) | NA |
| 032- | | | | | | |
| 076 | | | | | | |
| OGI- | LCA | NMNAT1 | NM_022787.3 | c.709C > T | p.(Arg237Cys) | 22842227 |
| 007- | | | | | | |
| 017 | | | | | | |
| OGI- | RP | RPGR | NM_001034853.1 | NA | NA | NA |
| 089- | | | | | | |
| 222 | | | | | | |
| OGI- | Star | ABCA4 | NM_000350.2 | c.4539 + 2028C > T | splicing defect | 23918662 |
| 147- | | | | | | |
| 394 | | | | | | |
| OGI- | U | USH2A | NM_206933.2 | c.7595-2144A > G | splicing defect | 22009552 |
| 387- | | | | | | |
| 839 | | | | | | |

### Example 11. Improved Diagnoses

Of note, seven of the subjects studied were found to have mutations in genes that are not primarily associated with their phenotypes (denoted with * in Table 7). Specific examples include cone dystrophy due to mutations in the ORF15 region of RPGR, NRL mutations in Chorio-retinal atrophy, and TMEM67 mutations in Senior-Loken syndrome. The following are some examples.

OGI-035-090. Subject OGI-035-090 is a male proband with a clinical diagnosis of cone dysfunction syndrome. Clinical history is significant for central vision loss and photophobia, with bull's eye patterns detected on fundus exam, and ERG analyses showing cone dysfunction with preserved rod function (FIGS. 5A-F). GEDi genetic diagnostic testing identified a mutation in the 3′ end of the ORF15 region of the RPGR gene; no other likely cause of disease was identified (Table 6). Although RPGR was originally identified as an RP disease gene, cases of cone dystrophy due to mutations in the ORF15 region of RPGR have been reported¹⁵⁻¹⁸.

OGI-019-047. OGI-019-047 is a female who presented at age 5 with peripheral field loss and nyctalopia. Fundus exam showed regions of chorioretinal atrophy near the vascular arcades and optic nerves (FIGS. 5A-F). A potential diagnosis of gyrate atrophy was suggested by these clinical findings, but plasma amino acid screening revealed normal ornithine levels, and sequencing of the OAT gene, as well as the CHM and CRB1 genes, was negative. GEDi sequencing identified a homozygous null mutation in NRL gene (Table 6). Segregation analysis confirmed biparental inheritance. Although mutations in NRL are predominantly associated with RP, null mutations at the same location in this gene have been reported as a cause of chorioretinal atrophy¹⁹.

OGI-314-749. This 31-year old subject has a history of renal disease diagnosed at age 12, and developed renal failure at age 14. He had onset of nyctalopia and decreased peripheral vision in his 20s, leading to the diagnosis of RP at age 25. He was followed regularly for both renal disease and retinal disease, but the potential diagnosis of Senior-Loken syndrome was not considered until he was evaluated at age 31 by a retinal degeneration specialist. The exam showed retinal degenerative changes consistent with RP (FIGS. 5A-F). GEDi genetic diagnostic testing identified mutations in TMEM67, consistent with a ciliopathy (Table 6). Mutations in TMEM67 have been reported to be associated with several cilia-associated disorders, including Meckel syndrome, Joubert syndrome, Bardet-Biedl syndrome and nephronophthisis (MIM: 609884)^{20-24.} Mutations in TMEM67 have not been reported previously in patients with Senior-Loken syndrome.

### REFERENCES:

1. Calvo S E, Compton A G, Hershman S G, et al. Molecular diagnosis of infantile mitochondrial disease with targeted next-generation sequencing. Sci Transl Med. Jan. 25, 2012; 4(118):118ra110.
2. Sommen M, Van Camp G. Genetic diagnostics of early childhood hearing loss: better testing with next-generation DNA sequencing. B-Ent. 2013; Suppl 21:51-56.
3. Rehm H L. Disease-targeted sequencing: a cornerstone in the clinic. Nat Rev Genet. April 2013; 14(4):295-300.
4. Pugh T J, Kelly M A, Gowrisankar S, et al. The landscape of genetic variation in dilated cardiomyopathy as surveyed by clinical DNA sequencing. Genetics In Medicine. Feb. 6, 2014.
5. Cottrell C E, Al-Kateb H, Bredemeyer A J, et al. Validation of a next-generation sequencing assay for clinical molecular oncology. Journal Molecular Diagnostics. January 2014; 16(1):89-105.
6. Levenson D. Whole-exome sequencing emerges as clinical diagnostic tool: Testing method proves useful for diagnosing wide range of genetic disorders. American Journal Medical Genetics. January 2014; 164(1):ix-x.
7. Gargis A S, Kalman L, Berry M W, et al. Assuring the quality of next-generation sequencing in clinical laboratory practice. Nature Biotechnology. November 2012; 30(11):1033-1036.

8. Rehm H L, Bale S J, Bayrak-Toydemir P, et al. ACMG clinical laboratory standards for next-generation sequencing. Genetics In Medicine. September 2013; 15(9):733-747.
9. Yang Y, Muzny D M, Reid J G, et al. Clinical whole-exome sequencing for the diagnosis of mendelian disorders. New England Journal Medicine. Oct. 17, 2013; 369(16):1502-1511.
10. Shearer A E, Black-Ziegelbein E A, Hildebrand M S, et al. Advancing genetic testing for deafness with genomic technology. Journal Medical Genetics. September 2013; 50(9):627-634.
11. Buch H, Vinding T, La C M, Appleyard M, Jensen G B, Nielsen N V. Prevalence and causes of visual impairment and blindness among 9980 Scandinavian adults: the Copenhagen City Eye Study. Ophthalmology. 2004; 111(1):53-61.
12. Congdon N, O'Colmain B, Klaver C C, et al. Causes and prevalence of visual impairment among adults in the United States. Archives Ophthalmology. April 2004; 122(4):477-485.
13. Quigley H A, Broman A T. The number of people with glaucoma worldwide in 2010 and 2020. British Journal Ophthalmology. March 2006; 90(3):262-267.
14. Fan B J, Wiggs J L. Glaucoma: genes, phenotypes, and new directions for therapy. Journal Clinical Investigation. September 2010; 120(9):3064-3072.
15. Gemenetzi M, Yang Y, Lotery A J. Current concepts on primary open-angle glaucoma genetics: a contribution to disease pathophysiology and future treatment. Eye. March 2012; 26(3):355-369.
16. Neuhann T, Rautenstrauss B. Genetic and phenotypic variability of optic neuropathies. Expert Review Neurotherapeutics. April 2013; 13(4):357-367.
17. Maguire A M, Simonelli F, Pierce E A, et al. Safety and efficacy of gene transfer for Leber's congenital amaurosis. New England Journal of Medicine. May 22, 2008; 358(21):2240-2248.
18. Bainbridge J W, Smith A J, Barker S S, et al. Effect of gene therapy on visual function in Leber's congenital amaurosis. New England Journal of Medicine. 2008; 358(21):2231-2239.
19. Cideciyan A V, Aleman T S, Boye S L, et al. Human gene therapy for RPE65 isomerase deficiency activates the retinoid cycle of vision but with slow rod kinetics. Proceedings National Academy Sciences USA. Sep. 30, 2008; 105(39):15112-15117.
20. MacLaren R E, Groppe M, Barnard A R, et al. Retinal gene therapy in patients with choroideremia: initial findings from a phase 1/2 clinical trial. Lancet. Mar. 29, 2014; 383(9923):1129-1137.
21. Wiggs J L, Pierce E A. Genetic testing for inherited eye disease: who benefits? JAMA Ophthaltnol. October 2013; 131(10):1265-1266.
22. Song J, Smaoui N, Ayyagari R, et al. High-throughput retina-array for screening 93 genes involved in inherited retinal dystrophy. Investigative Ophthalmology & Visual Science. 2011; 52(12):9053-9060.
23. Audo I, Bujakowska K M, Leveillard T, et al. Development and application of a next-generation-sequencing (NGS) approach to detect known and novel gene defects underlying retinal diseases. Orphanet Journal Rare Diseases. 2012; 7:8.
24. Neveling K, Collin R W, Gilissen C, et al. Next-generation genetic testing for retinitis pigmentosa. Human Mutation. June 2012; 33(6):963-972.
25. Eisenberger T, Neuhaus C, Khan A O, et al. Increasing the Yield in Targeted Next-Generation Sequencing by Implicating CNV Analysis, Non-Coding Exons and the Overall Variant Load: The Example of Retinal Dystrophies. PLoS One. 2013; 8(11):e78496.
26. Wang F, Wang H, Tuan H F, et al. Next generation sequencing-based molecular diagnosis of retinitis pigmentosa: identification of a novel genotype-phenotype correlation and clinical refinements. Human Genetics. March 2014; 133(3):331-345.
27. Falk M J, Pierce E A, Consugar M, et al. Mitochondrial disease genetic diagnostics: optimized whole-exome analysis for all MitoCarta nuclear genes and the mitochondrial genome. Discov Med. December 2012; 14(79):389-399.
28. Falk M J, Zhang Q, Nakamaru-Ogiso E, et al. NMNAT1 mutations cause Leber congenital amaurosis. Nature Genetics. Jul. 29, 2012; 44(9):1040-1045.
29. den Hollander A I, Koenekoop R K, Yzer S, et al. Mutations in the CEP290 (NPHP6) gene are a frequent cause of Leber congenital amaurosis. American Journal of Human Genetics. September 2006; 79(3):556-561.
30. Webb T R, Parfitt D A, Gardner J C, et al. Deep intronic mutation in OFD1, identified by targeted genomic next-generation sequencing, causes a severe form of X-linked retinitis pigmentosa (RP23). Human Molecular Genetics. Aug. 15, 2012; 21(16):3647-3654.
31. Vache C, Besnard T, le Berre P, et al. Usher syndrome type 2 caused by activation of an USH2A pseudoexon: implications for diagnosis and therapy. Human Mutation. January 2012; 33(1):104-108.
32. Viera A J, Garrett J M. Understanding interobserver agreement: the kappa statistic. Family Medicine. May 2005; 37(5):360-363.
33. Braun T A, Mullins R F, Wagner A H, et al. Non-exomic and synonymous variants in ABCA4 are an important cause of Stargardt disease. Human Molecular Genetics. Dec. 20, 2013; 22(25):5136-5145.
34. Redin C, Le Gras S, Mhamdi O, et al. Targeted high-throughput sequencing for diagnosis of genetically heterogeneous diseases: efficient mutation detection in Bardet-Biedl and Alstrom syndromes. Journal Medical Genetics. August 2012; 49(8):502-512.
35. Flintoft L. Clinical genetics: exomes in the clinic. Nat Rev Genet. December 2013; 14(12):824.
36. Delanty N, Goldstein D B. Diagnostic exome sequencing: a new paradigm in neurology. Neuron. Nov. 20, 2013; 80(4):841-843.
37. Dorschner M O, Amendola L M, Turner E H, et al. Actionable, pathogenic incidental findings in 1,000 participants' exomes. American Journal Human Genetics. Oct. 3, 2013; 93(4):631-640.
38. Platt J, Cox R, Enns G M. Points to Consider in the Clinical Use of NGS Panels for Mitochondrial Disease: An Analysis of Gene Inclusion and Consent Forms. Journal Genetic Counseling. Jan. 8, 2014.
39. Berglund E C, Lindqvist C M, Hayat S, et al. Accurate detection of subclonal single nucleotide variants in whole genome amplified and pooled cancer samples using HaloPlex target enrichment. BMC Genomics. 2013; 14:856.
40. Fromer M, Moran I L, Chambert K, et al. Discovery and statistical genotyping of copy-number variation from whole-exome sequencing depth. American Journal Human Genetics. Oct. 5, 2012; 91(4):597-607.

Table 11. Bait sequences, identified by SEQ ID NO:, their corresponding genomic coordinates, and the gene to which the bait is localized.

Lengthy table referenced here
US20180346981A1-20181206-T00001
Please refer to the end of the specification for access instructions.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. For example, although the methods are described for use in detecting variations associated with genetic eye diseases, other genetic variations can also be detected, e.g., variations associated with cancer.

LENGTHY TABLES
The patent application contains a lengthy table section. A copy of the table is available in electronic form from the USPTO web site (http://seqdata.uspto.gov/?pageRequest=docDetail&DocID=US20180346981A1). An electronic copy of the table will also be available from the USPTO upon request and payment of the fee set forth in 37 CFR 1.19(b)(3).

1. A method of detecting a genetic variation in a genomic region associated with a genetic eye disease in a subject, the method comprising:
contacting a sample comprising fragmented genomic DNA (gDNA) from the subject with a plurality of bait ribonucleotides, wherein each bait binds to a fragment of gDNA comprising a genomic target sequence that is within a genomic region associated with a genetic eye disease; the plurality of baits comprises sufficient baits to sequence each target sequence is sequenced at least 10 times; and the plurality comprises baits that bind to mutations in at least 100 genes;
enriching the sample for the bait/gDNA complexes;
isolating the gDNA fragments;
determining the sequences of the isolated gDNA fragments using next generation sequencing,
wherein each selected mutation is sequenced at least 10 times; and
comparing the determined sequences to corresponding reference sequences, thereby detecting the presence of genetic variations in a genomic region associated with genetic eye disease in the subject.

2. The method of claim 1, wherein the plurality of bait oligonucleotides comprises at least 10,000 oligonucleotides.

3. The method of claim 1, wherein the mutations include one or more mutations listed in Tables 2, 3, 4, 9, or 10.

4. The method of claim 1, wherein the reference sequences include the normal sequence, and wherein differences between the subject's genomic sequences and normal reference sequences indicate the presence of mutations, and identity between the subject's genomic sequences and normal reference sequences indicate the absence of mutations.

5. The method of claim 1, wherein the reference sequences include the mutant sequences, preferably comprising a known mutation associated with eye disease, and wherein identity between the subject's genomic sequences and mutant reference sequences indicate the presence of mutations, and differences between the subject's genomic sequences and mutant reference sequences indicate the absence of mutations.

6. The method of claim 1, wherein sequence of each selected mutation at least 10 times is obtained by one or more of: (1) stochastically increasing the number of baits per target; (2) near-target capture, using bait sequences up to 75 bp away from the mutation of interest; and/or (3) tiling the baits such that numerous overlapping baits target the same region.

7. A kit for use in detecting a mutation in a genomic region associated with a genetic eye disease in a subject, the kit comprising:
a plurality of bait ribonucleotides, wherein each bait is complementary to a genomic target sequence that is within a genomic region associated with a genetic eye disease; the plurality of baits comprises sufficient baits to sequence each target sequence is sequenced at least 10 times; and the plurality comprises baits that bind to regions in at least 100 genes;
reagents for enriching the sample for the bait/gDNA complexes;
reagents for isolating the gDNA fragments; and
reagents for determining the sequences of the isolated gDNA fragments using next generation sequencing.

8. The kit of claim 7, wherein the plurality of bait oligonucleotides comprises at least 10,000 oligonucleotides.

9. The kit of claim 8, wherein the plurality of bait oligonucleotides comprises at least 10,000 oligonucleotides listed in Table 11.

10. The kit of claim 7, wherein the target regions include one or more genes or regions listed in Tables 2, 3, 4, 9, or 10.

11. The kit of claim 10, wherein the target regions include 50 or more genes or regions listed in Tables 2, 3, 4, 9, or 10.

12. The method of claim 2, wherein the plurality of bait oligonucleotides comprises at least 10,000 oligonucleotides listed in Table 11.

13. The method of claim 1, wherein the subject is a mammalian subject.

14. The method of claim 13, wherein the subject is a human.
